(19) United States
(12) Patent Application Publication
(10) Pub. No.: US 2020/0354413 A1
(43) Pub. Date: Nov. 12, 2020

(54) STABILIZED PEPTIDE-MEDIATED TARGETED PROTEIN DEGRADATION

(71) Applicant: Dana-Farber Cancer Institute, Inc., Boston, MA (US)

(72) Inventors: Loren D. Walensky, Newton, MA (US); Gregory H. Bird, Pelham, NH (US); Ann Morgan Cathcart, Cambridge, MA (US); Rida Mourtada, Brookline, MA (US); Henry D. Herce, Brookline, MA (US); James E. Bradner, Weston, MA (US)

(21) Appl. No.: 16/766,083

(22) PCT Filed: Dec. 14, 2018
(86) PCT No.: PCT/US2018/065784

§ 371 Date: May 21, 2020

Related U.S. Application Data
(60) Provisional application No. 62/599,608, filed on Dec. 15, 2017.

(51) Int. Cl. C07K 14/435; A61K 47/54

(52) U.S. Cl. CPC C07K 14/435; C07K 2319/00; A61K 47/545

(57) ABSTRACT

The present application describes stapled peptide degron chimeras, which act as protein degradation inducing moieties, either by combining a stapled peptide that binds a disease-related protein with a small molecule degron, such as a cereblon- or VHL-binding small molecule as the degron, or a polypeptide sequence degron, such as a Cop1-binding Trib peptide as the degron; or by combining a stapled peptide degron with a peptide, such as a stapled peptide, or a small molecule that binds a disease-related protein. The present application also relates to methods for the targeted degradation of endogenous proteins through the use of stapled peptide degron chimeras which can be utilized in the treatment of proliferative disorders or other conditions whereby elimination of a disease-causing or disease-related protein would have a therapeutic benefit. The present application also provides methods for making compounds of the application and intermediates thereof.

### CROSS REFERENCE TO RELATED APPLICATIONS

This application claims priority to U.S. Provisional Application No. 62/599,608, filed Dec. 15, 2017, which is incorporated by reference herein in its entirety.

### TECHNICAL FIELD

This disclosure relates to stabilized peptide and small molecule chimeric compounds, termed stapled peptide-degron chimeras, that act as combined protein targeting (by the stapled peptide or molecular portion) and protein degradation-inducing moieties (by another stapled peptide or molecular portion). The chimeras include a stapled peptide fused to either: (i) a small molecule degron (e.g., a cereblon- or VHL-binding small molecule as the degron), (ii) a polypeptide sequence degron, (iii) a stapled peptide as the degron, or (iv) a small molecule as the protein targeting compound (with the stapled peptide serving as the degron), and methods of their use.

### BACKGROUND

A degron is a portion of a protein that plays a major role in its degradation. Degrons are usually short amino acid sequences that can be located anywhere in the protein sequence (Cho et al., Genes & Development, 24 (5): 438-442 (2010); Fortmann et al., Journal of Molecular Biology, 427 (17): 2748-2756 (2015); Dohmen et al., Science, 263(5151):1273-1276 (1994); Varshavsky, Proceedings of the National Academy of Sciences, 93 (22):12142-12149 (1996)). Some proteins, in fact, have multiple degrons. Degrons have been identified both in prokaryotes and eukaryotes. Although there are several types of degrons, and despite the fact that there is a high degree of variability within these groups, degrons are all similar for their involvement in regulating the rate of a protein's degradation. The degradation may involve ubiquitin or may be ubiquitin-independent. Degrons that are ubiquitin-dependent contain a specific sequence that is recognized by cognate ubiquitin E3 ligases.

The Ubiquitin-Proteasome Pathway (UPP) is a major pathway that regulates key regulator proteins and degrades misfolded or abnormal proteins. The covalent attachment of ubiquitin to specific protein substrates is achieved through the action of E3 ubiquitin ligases. For example, Cereblon (CRBN) interacts with damaged DNA binding protein 1 (DDB1) and forms an E3 ubiquitin ligase complex with Cullin 4 (CUL4A), which then functions as a substrate receptor such that proteins recognized by CRBN can be ubiquitinated and degraded by the proteasome. CRBN has also been identified to bind immunomodulatory drugs (IMiDs), such as thalidomide. Such binding has been associated with the mechanism of teratogenicity and also the cytotoxicity of IMiDs, such as lenalidomide, which is used to treat multiple myeloma.

### SUMMARY

This disclosure relates to the synthesis and characterization of bifunctional stabilized peptide-small molecule (e.g., thalidomide degron) conjugates, stabilized peptide-peptide (e.g., primary degron sequence) conjugates, stabilized peptide-stabilized peptide (e.g. primary degron sequence) conjugates, and small molecule-stabilized peptide (e.g. primary degron sequence) conjugates that can be used to target any protein of interest (these conjugates are also referred to as “chimeras”). For example, these conjugates are useful to target proteins that are involved in or that are causative of disease. The targeted proteins can be of viral, bacterial, animal, or human origin. In certain instances, the conjugates are useful to target disease causing or disease-related proteins. Such stabilized peptide conjugates are useful for treating diseases driven by such pathologic proteins. The capacity of stapled peptides to target large and typically undruggable protein interaction surfaces, coupled with a degron functionality, broadens the utility of degron technology beyond the reach of small molecules, and can enhance the biological activity of stapled peptides.

In a first aspect, the disclosure features a peptide-small molecule fusion comprising a protein-targeting stapled peptide and a small molecule degron moiety (e.g., a thalidomide moiety or a Von Hippel-Lindau (VHL) moiety).

In some embodiments, the small molecule degron (e.g., thalidomide moiety or VHL moiety) is conjugated to the N-terminus of the protein-targeting stapled peptide. In some instances, the small molecule degron (e.g., thalidomide moiety or VHL moiety) is conjugated to the C-terminus of the protein-targeting stapled peptide. In certain instances, the small molecule degron (e.g., thalidomide moiety or VHL moiety) is contained within a non-natural amino acid inserted in the peptide sequence between the N- and C-terminus of the protein-targeting stapled peptide. In some instances, the stapled peptide binds a disease-causing protein. In some instances, the stapled peptide binds an intracellular protein. In some instances, the stapled peptide binds an extracellular protein. In some instances, the stapled peptide binds a cell surface protein (e.g., a receptor). In some instances, the stapled peptide binds a killer protein (e.g., BAX, BAK) or a protein that is damaging to cells or that causes neurodegeneration (e.g., IgG, beta-amyloid, tau, α-synuclein, TDP-43, hemoglobin (sickle cell), superoxide dismutase, Notch3, FUS, GFAP). In some instances, the stapled peptide binds a protein selected from the group consisting of BCL2, BCLXL, MCL-1, BFL-1, BCL-w, BCL-B, EZH2, HDM2/HDMX, KRAS/NRAS/HRAS, MYC, b-catenin, PI3K, PTEN, TSC, AKT, BRCA1/2, EWS-FLI, MLL fusions, a receptor Tyrosine kinases, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In some instances, the stapled peptide binds a bacterial protein. In some instances, the stapled peptide binds a viral protein. In certain instances, the thalidomide moiety comprises the structure provided below:

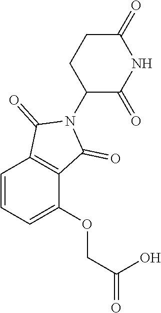 [SMILES: O=C(O)COc1cccc2c1C(=O)N(C1CCC(=O)NC1=O)C2=O]

In some instances, the thalidomide moiety, when conjugated at the N-terminus of the stabilized peptide, comprises the structure provided below:

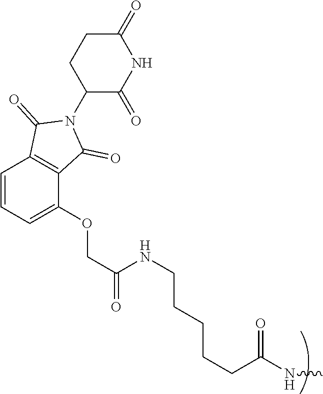

In some instances, the thalidomide moiety, when conjugated at the C-terminus of the stabilized peptide comprises the structure provided below:

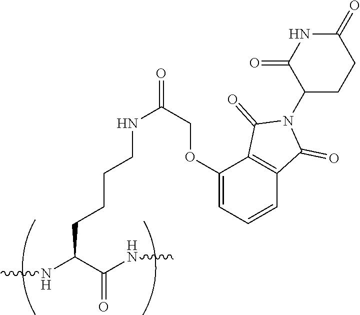

In some instances, the VHL moiety comprises the structure below:

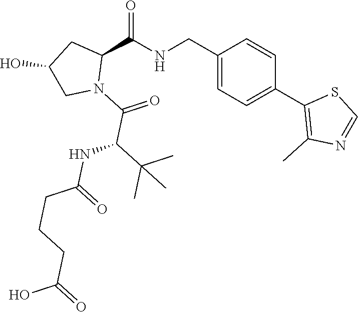 [SMILES: Cc1ncsc1-c1ccc(CNC(=O)[C@@H]2C[C@@H](O)CN2C(=O)[C@@H](NC(=O)CCCC(=O)O)C(C)(C)C)cc1]

In some instances, the VHL moiety comprises the structure below:

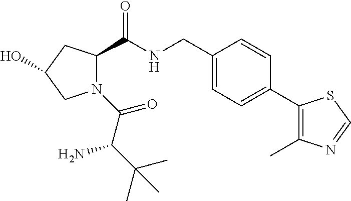 [SMILES: Cc1ncsc1-c1ccc(CNC(=O)[C@@H]2C[C@@H](O)CN2C(=O)[C@@H](N)C(C)(C)C)cc1]

In a second aspect, the disclosure features methods of treating a disease or disorder driven by a pathologic peptide or protein in a human subject in need thereof. The method comprises administering to the human subject a therapeutically effective amount of the peptide small molecule fusion described herein.

In a third aspect, the disclosure features a peptide degron that binds a WD40-repeat protein, wherein the WD40-repeat protein is a substrate adaptor for an E3 ubiquitin ligase. The peptide comprises a modified version of a natural binding sequence or a natural binding consensus sequence of an amino acid sequence that binds to the WD40-repeat protein. The modified version comprises at least one amino acid substitution, at least one amino acid deletion, at least one amino acid insertion, or any combination thereof within the natural binding consensus sequence of the amino acid sequence that binds to the WD40-repeat protein. Exemplary peptides comprising a modified version of a natural binding sequence or a natural binding consensus sequence of an amino acid sequence that binds to the WD40-repeat protein are provided in SEQ ID NOs.: 26-30 and 106-118. In one illustrative example, this disclosure provides a peptide that binds Constitutive Photomorphogenic 1 (Cop1) protein. The peptide comprises a modified version of the amino acid sequence DQIVPEY (SEQ ID NO:25). The modified version comprises at least one amino acid substitution, at least one amino acid deletion, at least one amino acid insertion, or any combination thereof in SEQ ID NO:25. If the modified version consists of a single amino acid substitution, then the amino acid substitution is not to an A or R at any one of positions 1 to 7 of SEQ ID NO:25, or to V at position 4 of SEQ ID NO:25.

In some embodiments, the peptide comprises the amino acid sequence set forth in SEQ ID NO:25 except having at least one amino acid substitution. In some instances, the peptide comprises the amino acid sequence set forth in SEQ ID NO:25 except having at least one amino acid deletion. In some instances, the peptide comprises the amino acid sequence set forth in SEQ ID NO:25 except having at least one amino acid substitution and at least one amino acid deletion. In certain instances, the peptide comprises the amino acid sequence set forth in SEQ ID NO:25 except having one to six amino acid substitutions. In some instances, position 4 (V) and/or position 5 (P) of SEQ ID NO:25 are not substituted. In some instances, one or more of positions 1 (D), position 2 (Q), position 3 (I), and position 6 (E) of SEQ ID NO:25 are substituted. In some instances, the peptide comprises the amino acid sequence set forth in SEQ ID NO:25 except having one amino acid deletion. In certain instances, position 7 (Y) of the amino acid sequence set forth in SEQ ID NO:25 is deleted. In some instances, the peptide comprises the amino acid sequence set forth in SEQ ID NO:25 except having with one to six amino acid substitutions and at least one amino acid deletion. In some instances, the peptide has an amino acid sequence selected from the group consisting of SEQ ID NOs.: 26 to 30. In one instance, the peptide has the amino acid sequence set forth in SEQ ID NO:30.

In certain embodiments, the peptide is 4 to 10 amino acids in length.

In some instances, the peptide binds Cop1 with a binding affinity of 1 nM to 300 nM. In some instances, the peptide binds Cop1 with a binding affinity of 1 nM to 1000 nM. In some instances, the peptide binds Cop1 with a binding affinity of 10 nM to 300 nM. In some instances, the peptide binds Cop1 with a binding affinity of 100 nM to 300 nM. In some instances, the peptide binds Cop1 with a binding affinity of 200 nM to 300 nM. In some instances, the peptide binds Cop1 with a binding affinity of 200 nM to 1000 nM.

In a fourth aspect, the disclosure relates to a chimeric fusion polypeptide comprising a protein-targeting stapled peptide and a Trib1 peptide degron or variant thereof.

In some embodiments, the stapled peptide binds an intracellular protein. In some embodiments, the stapled peptide binds an extracellular protein. In some embodiments, the stapled peptide binds a cell surface protein (e.g., receptor). In some embodiments, the stapled peptide binds a disease-causing or disease-related protein. In some embodiments, the stapled peptide binds a killer protein (e.g., BAX, BAK) or a protein that is damaging to cells or that causes neurodegeneration (e.g., IgG, beta-amyloid, tau, α-synuclein, TDP-43, HbS, superoxide dismutase, Notch3, FUS, GFAP). In some embodiments, the stapled peptide binds a protein selected from the group consisting of BCL2, BCLXL, MCL-1, BFL-1, BCL-w, BCL-B, EZH2, HDM2/HDMX, KRAS/NRAS/HRAS, MYC, 0-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In some embodiments, the stapled peptide binds a bacterial protein. In some embodiments, the stapled peptide binds a viral protein.

In a fifth aspect, the disclosure features a chimeric polypeptide comprising a stapled peptide and a peptide that binds a WD40-repeat protein, wherein the WD40-repeat protein is a substrate adaptor for an E3 ubiquitin ligase. The peptide comprises a modified version of the natural binding sequence or the natural binding consensus sequence of an amino acid sequence that binds to the WD40-repeat protein. The modified version comprises at least one amino acid substitution, at least one amino acid deletion, at least one amino acid insertion, or any combination thereof within the natural binding consensus sequence of the amino acid sequence that binds to the WD40-repeat protein.

In some embodiments, the WD40-repeat protein that is a substrate adaptor for an E3 ubiquitin ligase is selected from the group consisting of MDM2, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1.

In some embodiments, the natural binding sequence or the natural binding consensus sequence is a sequence selected from the group consisting of SEQ ID NOs.: 25, 31-46, and 65-105. In some instances, the natural binding consensus sequence is SEQ ID NO:25. In other instances, the natural binding consensus sequence is SEQ ID NO:46.

In some embodiments, the peptide comprises the amino acid sequence set forth in SEQ ID NO:25 except having at least one amino acid substitution. In some instances, the peptide comprises the amino acid sequence set forth in SEQ ID NO:25 except having at least one amino acid deletion. In some instances, the peptide comprises the amino acid sequence set forth in SEQ ID NO:25 except having at least one amino acid substitution and at least one amino acid deletion. In certain instances, the peptide comprises the amino acid sequence set forth in SEQ ID NO:25 except having one to six amino acid substitutions. In some instances, position 4(V) and/or position 5 (P) of SEQ ID NO:25 are not substituted. In some instances, one or more of positions 1(D), position 2 (Q), position 3 (I), and position 6 (E) of SEQ ID NO:25 are substituted. In some instances, the peptide comprises the amino acid sequence set forth in SEQ ID NO:25 except having one amino acid deletion. In certain instances, position 7 (Y) of the amino acid sequence set forth in SEQ ID NO:25 is deleted. In some instances, the peptide comprises the amino acid sequence set forth in SEQ ID NO:25 except having with one to six amino acid substitutions and at least one amino acid deletion. In some instances, the peptide has an amino acid sequence selected from the group consisting of SEQ ID NOs.: 26 to 30. In one instance, the peptide has the amino acid sequence set forth in SEQ ID NO:30.

In certain embodiments, the peptide is 4 to 30 amino acids in length. In certain embodiments, the peptide is 4 to 20 amino acids in length. In certain embodiments, the peptide is 4 to 15 amino acids in length. In certain embodiments, the peptide is 5 to 20 amino acids in length.

In certain embodiments, the peptide binds Cop1 with a binding affinity of 1 nM to 300 nM; 10 nM to 300 nM; 100 nM to 300 nM; or 200 nM to 300 nM. In certain embodiments, the peptide binds Cop1 with a binding affinity of 1 nM to 1000 nM. In certain embodiments, the peptide binds Cop1 with a binding affinity of 200 nM to 1000 nM.

In some embodiments, the stapled peptide binds an intracellular protein. In some embodiments, the stapled peptide binds an extracellular protein. In some embodiments, the stapled peptide binds a cell surface protein (e.g., receptor). In some embodiments, the stapled peptide binds a disease-causing or disease-related protein. In some embodiments, the stapled peptide binds a killer protein (e.g., BAX, BAK) or a protein that is damaging to cells or that causes neurodegeneration (e.g., IgG, beta-amyloid, tau, α-synuclein, TDP-43, HbS (hemoglobin-sickle cell), superoxide dismutase, Notch3, FUS, GFAP). In some embodiments, the stapled peptide binds a protein selected from the group consisting of BCL2, BCLXL, MCL-1, BFL-1, BCL-w, BCL-B, EZH2, HDM2/HDMX, KRAS/NRAS/HRAS, MYC, β-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In some embodiments, the stapled peptide binds a bacterial protein. In some embodiments, the stapled peptide binds a viral protein. In certain instances, the stapled peptide targets a protein aggregate (e.g., beta-amyloid) that causes neurodegeneration.

In a sixth aspect, the disclosure features a modified protein of a first protein that comprises a structurally disordered region. The modified protein differs from the first protein in that the structurally disordered region comprises a peptide that binds a WD40-repeat protein that is a substrate adaptor for an E3 ubiquitin ligase. The peptide comprises a modified version of the natural binding consensus sequence, wherein the modified version comprises at least one amino acid substitution, at least one amino acid deletion, at least one amino acid insertion, or any combination thereof within the natural binding consensus sequence.

In certain embodiments, the WD40-repeat protein that is a substrate adaptor for an E3 ubiquitin ligase is selected from the group consisting of MDM2, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1. In some instances, the natural binding consensus sequence is a sequence selected from the group consisting of SEQ ID NOs.: 25, 31-46, and 65-105. In some instances, the natural binding consensus sequence is SEQ ID NO:25. In some instances, the natural binding consensus sequence is SEQ ID NO:46.

In some embodiments, the peptide comprises the amino acid sequence set forth in SEQ ID NO:25 except having at least one amino acid substitution. In some instances, the peptide comprises the amino acid sequence set forth in SEQ ID NO:25 except having at least one amino acid deletion. In some instances, the peptide comprises the amino acid sequence set forth in SEQ ID NO:25 except having at least one amino acid substitution and at least one amino acid deletion. In certain instances, the peptide comprises the amino acid sequence set forth in SEQ ID NO:25 except having one to six amino acid substitutions. In some instances, position 4(V) and/or position 5 (P) of SEQ ID NO:25 are not substituted. In some instances, one or more of positions 1(D), position 2 (Q), position 3 (I), and position 6 (E) of SEQ ID NO:25 are substituted. In some instances, the peptide comprises the amino acid sequence set forth in SEQ ID NO:25 except having one amino acid deletion. In certain instances, position 7 (Y) of the amino acid sequence set forth in SEQ ID NO:25 is deleted. In some instances, the peptide comprises the amino acid sequence set forth in SEQ ID NO:25 except having with one to six amino acid substitutions and at least one amino acid deletion. In some instances, the peptide has an amino acid sequence selected from the group consisting of SEQ ID NOs.: 26 to 30. In one instance, the peptide has the amino acid sequence set forth in SEQ ID NO:30.

In certain embodiments, the peptide is 4 to 10 amino acids in length.

In certain embodiments, the peptide binds Cop1 with a binding affinity of 1 nM to 300 nM; 10 nM to 300 nM; 100 nM to 300 nM; or 200 nM to 300 nM. In certain embodiments, the peptide binds Cop1 with a binding affinity of 1 nM to 1000 nM. In certain embodiments, the peptide binds Cop1 with a binding affinity of 200 nM to 1000 nM.

In a seventh aspect, the disclosure features a method of treating a disease or disorder driven by a pathologic peptide or protein in a human subject in need thereof. The method comprising administering to the human subject a therapeutically effective amount of the chimeric fusion polypeptide described herein.

In an eighth aspect, the disclosure features a peptide degron selected from the group consisting of SEQ ID NOs.: 106 to 118.

In some embodiments, these peptides are linked to a stabilized peptide.

In a ninth aspect, the disclosure provides a stabilized peptide-peptide degron chimera selected from the group consisting of SEQ ID NOs.: 119 to 126.

In certain embodiments of all the above aspects, the stabilized peptide-degron chimera is used to target the degradation of any one or more of BCL2, BCLXL, BCLw, MCL-1, BFL-1, BAX, MDM2, or MDMX.

In a tenth aspect, the disclosure provides a stabilized peptide-stabilized peptide degron chimera, which comprises two stabilized peptides—a first stabilized peptide and a second stabilized peptide—wherein the first stabilized peptide binds to a first protein which is the protein target to be degraded, and the second stabilized peptide binds to a second protein which is a degrader protein. In certain embodiments, the first stabilized peptide binds a disease-related protein, which is the target of degradation, and the second stabilized peptide binds to a degrader protein, such an E3 ligase (e.g. MDM2).

In some embodiments, the first protein is an intracellular protein. In some embodiments, the first protein is an extracellular protein. In some embodiments, the first protein is a cell surface protein (e.g., receptor). In some embodiments, the first protein is a disease-causing or disease-related protein. In some embodiments, the first protein is a killer protein (e.g., BAX, BAK) or a protein that is damaging to cells or that causes neurodegeneration (e.g., IgG, beta-amyloid, tau, α-synuclein, TDP-43, HbS (hemoglobin-sickle cell), superoxide dismutase, Notch3, FUS, GFAP). In some embodiments, the first protein is a protein selected from the group consisting of BCL2, BCLXL, MCL-1, BFL-1, BCL-w, BCL-B, EZH2, HDM2/HDMX, KRAS/NRAS/HRAS, MYC, β-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In some embodiments the first protein is a bacterial protein. In some embodiments, the first protein is a viral protein. In certain instances, the first protein is a protein aggregate (e.g., beta-amyloid) that causes neurodegeneration.

In certain embodiments, the first stabilized peptide has an amino acid sequence set forth in any one of SEQ ID NOs.: 1-24, and 134, or a variant thereof. In certain embodiments, the second stabilized peptide has an amino acid sequence set forth in SEQ ID NO: 6, or a variant thereof. In certain embodiments, the second stabilized peptide has an amino acid sequence set forth in SEQ ID NO: 18, or a variant thereof.

In certain embodiments, the second protein is a degrader protein, such as, e.g., an E3 ubiquitin ligase or a substrate adaptor for an E3 ubiquitin ligase. In certain embodiments, the second protein is an E3 ubiquitin ligase. In some embodiments, the second protein binds to E3 ligase (e.g., MDM2) or a protein that is complexed to an E3 ligase, such as MDMX binding to MDM2. In certain embodiments, the E3 ubiquitin ligase is a RING E3 ubiquitin ligase (e.g., Mdm2-MdmX, TRIM5α, c-CBL, cIAP, RNF4, BIRC7, IDOL, BRCA1-BARD1, RING1B-Bmi1, E4B, CHIP, Prp19). In certain embodiments, the E3 ubiquitin ligase is a HECT E3 ubiquitin ligase (e.g., Smurf1, Smurf2, Itch, E6AP). In certain embodiments, the E3 ubiquitin ligase is a RBR E3 ubiquitin ligase (e.g., Parkin, Parc, RNF144 (A/B), HOIP, HHARI). See, e.g., Morreale and Walden, Cell 165, 2016 DOI http:/dx.doi.org/10.1016/j.cell.2016.03.003 for non-limiting examples of E3 ubiquitin ligases.

In certain embodiments, the second stabilized peptide portion of the chimera has an amino acid sequence set forth in SEQ ID NO: 134, or a variant thereof. In certain embodiments, the second stabilized peptide has an amino acid sequence set forth in SEQ ID NO: 6, or a variant thereof. In certain embodiments, the second stabilized peptide has an amino acid sequence set forth in SEQ ID NO: 18, or a variant thereof.

In an eleventh aspect, the disclosure provides a small molecule-stabilized peptide degron chimera, which comprises a small molecule and a stabilized peptide, wherein the small molecule binds to a first protein which is the protein target to be degraded, and the stabilized peptide binds to a second protein which is a degrader protein. In certain embodiments, the stabilized peptide binds to and recruits a degrader protein, such an E3 ligase (e.g. MDM2), or a degrader protein complex, such as the MDM2/MDMX complex.

In some embodiments, the first protein is an intracellular protein. In some embodiments, the first protein is an extracellular protein. In some embodiments, the first protein is a cell surface protein (e.g., receptor). In some embodiments, the first protein is a disease-causing or disease-related protein. In some embodiments, the first protein is a killer protein (e.g., BAX, BAK) or a protein that is damaging to cells or that causes neurodegeneration (e.g., IgG, beta-amyloid, tau, α-synuclein, TDP-43, HbS (hemoglobin-sickle cell), superoxide dismutase, Notch3, FUS, GFAP). In some embodiments, the first protein is a protein selected from the group consisting of BCL2, BCLXL, MCL-1, BFL-1, BCL-w, BCL-B, EZH2, HDM2/HDMX, KRAS/NRAS/HRAS, MYC, 0-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In some embodiments the first protein is a bacterial protein. In some embodiments, the first protein is a viral protein. In certain instances, the first protein is a protein aggregate (e.g., beta-amyloid)

In certain embodiments, the second protein is a degrader protein, such as, e.g., an E3 ubiquitin ligase or a substrate adaptor for an E3 ubiquitin ligase. In certain embodiments, the second protein is an E3 ubiquitin ligase. In some embodiments, the second protein binds to E3 ligase (e.g., MDM2) or a protein that is complexed to an E3 ligase, such as MDMX binding to MDM2. In certain embodiments, the E3 ubiquitin ligase is a RING E3 ubiquitin ligase (e.g., Mdm2-MdmX, TRIM5α, c-CBL, cIAP, RNF4, BIRC7, IDOL, BRCA1-BARD1, RING1B-Bmi1, E4B, CHIP, Prp19). In certain embodiments, the E3 ubiquitin ligase is a HECT E3 ubiquitin ligase (e.g., Smurf1, Smurf2, Itch, E6AP). In certain embodiments, the E3 ubiquitin ligase is a RBR E3 ubiquitin ligase (e.g., Parkin, Parc, RNF144 (A/B), HOIP, HHARI). See, e.g., Morreale and Walden, Cell 165, 2016 DOI http:/dx.doi.org/10.1016/j.cell.2016.03.003 for non-limiting examples of E3 ubiquitin ligases.

In a twelfth aspect, the disclosure provides a chimera comprising: a first moiety attached to a second moiety; wherein the first moiety binds to a first protein, which is targeted for degradation, and the second moiety binds to a second protein; wherein the second protein is a protein degrader.

In certain aspects, the first moiety and second moiety are covalently attached to each other. In certain aspects, the first moiety and the second moiety are attached to each other via a linker.

In some embodiments, the first protein is an intracellular protein. In some embodiments, the first protein is an extracellular protein. In some embodiments, the first protein is a cell surface protein (e.g., receptor). In some embodiments, the first protein is a disease-causing or disease-related protein. In some embodiments, the first protein is a killer protein (e.g., BAX, BAK) or a protein that is damaging to cells or that causes neurodegeneration (e.g., IgG, beta-amyloid, tau, α-synuclein, TDP-43, HbS (hemoglobin-sickle cell), superoxide dismutase, Notch3, FUS, GFAP). In some embodiments, the first protein is a protein selected from the group consisting of BCL2, BCLXL, MCL-1, BFL-1, BCL-w, BCL-B, EZH2, HDM2/HDMX, KRAS/NRAS/HRAS, MYC, β-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In some embodiments the first protein is a bacterial protein. In some embodiments, the first protein is a viral protein. In certain instances, the first protein is a protein aggregate (e.g., beta-amyloid)

In certain instances, the first moiety comprises a first stapled peptide that binds to the first protein targeted for degradation. In certain instances, the first moiety comprises a small molecule that binds to the first protein targeted for degradation. In certain instances, the second moiety comprises a second stapled peptide that binds to the second protein, such as the protein degrader. In certain instances, the second moiety comprises a small molecule that binds to the second protein, such as the protein degrader. In certain instances, the second moiety comprises a peptide degron that binds to the protein degrader. In certain instances, the first moiety comprises a first stapled peptide that binds to the first protein and the second moiety comprises a second stapled peptide that binds to the second protein. In certain instances, the first moiety comprises a first stapled peptide that binds to the first protein and the second moiety comprises a small molecule that binds to the second protein. In certain instances, the first moiety comprises a first stapled peptide that binds to the first protein and the second moiety comprises a peptide degron that binds to the protein degrader. In certain instances, the first moiety comprises a small molecule that binds to the first protein and the second moiety comprises a stapled peptide that binds to the second protein.

In certain instances in which the first moiety is a stapled peptide, the stapled peptide does not comprise a Bcl-2 homology 3 (BH3) domain polypeptide. In certain instances in which the first moiety is a stapled peptide, the stapled peptide does not comprise: (a) a Bcl-2 homology 3 domain from MCL-1, (b) a MCL-1 stabilized alpha helix of BCL2 domain, or (c) MCL-1 SAHBD.

In certain instances in which the first moiety is a stapled peptide, the second moiety is attached to the N-terminus of the first moiety. In certain instances in which the first moiety is a stapled peptide, the second moiety is attached to the C-terminus of the first moiety. In certain instances in which the first moiety is a first stapled peptide, the second moiety is attached to an internal amino acid position of the first moiety.

In certain instances in which the second moiety is a stapled peptide, the first moiety is attached to the N-terminus of the second moiety. In certain instances in which the second moiety is a stapled peptide, the first moiety is attached to the C-terminus of the second moiety. In certain instances in which the second moiety is a stapled peptide, the first moiety is attached to an internal amino acid position of the second moiety.

In some aspects, the protein degrader degrades the first protein targeted for degradation.

In a thirteenth aspect, the disclosure provides a method of treating a disease or disorder driven by a pathologic peptide or protein in a subject in need thereof, the method comprising administering to the subject a therapeutically effective amount of the chimera described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 provides the peptide sequences and degron type/location on a series of representative stapled peptide sequences. The amino acid sequences in column 1 are assigned SEQ ID NOs.: 1-24. #=N-term Ac or degron Ahx, as denoted; %=C-term Lys(ivdde) or Lys(degron) as denoted; X=S-pentenyl alanine; 8=R-octenyl alanine; B=norleucine; * (within the amino acid sequence)=cyclobutylalanine (ivdde=1-(4,4-Dimethyl-2,6-dioxocyclohex-1-ylidene)-3-methylbutyl)

FIG. 2 shows the carboxy degron thalidomide moiety that was coupled to a resin bound primary amine to yield the stapled peptide degrons shown in FIG. 1.

FIG. 3 shows the C-terminal degron-containing moiety, which is a side chain conjugated lysine linkage to the peptide.

FIG. 4 shows the N-terminal degron-containing moiety, which is an aminohexanoic acid linkage to the peptide.

FIG. 5 shows the structure of diaminobutanoic acid (“DAB”) and the chemical structures for the THAL and VHL ligands used for coupling to acids or amines.

FIG. 6 shows the structures of various linkers (Gly, βAla, and Linkers 1-8).

FIG. 7 shows a series of stapled peptide degron chimeras whereby the diaminobutanoic acid is incorporated into the stapled peptide to attach the small molecule degron, and linkers of various compositions and length are installed to separate the stapled peptide from the small molecule or peptide degron (THAL, TRIB, or VHL). The stapled peptide sequences depicted in FIG. 7 are: IWIA%ELRXIGDXFNAYYARR (SEQ ID NO:127), IWIAQELRXIGDXFN%YYARR (SEQ ID NO:128), LTF8%YWAQLXSAA (SEQ ID NO:129), LTF8EYWAQLX%AA (SEQ ID NO:130); and %TF8EYWAQLXSAA (SEQ ID NO:131), wherein % is as indicated in the Figure, 8 is (R)-2-(7-octenyl)alanine, and X is (S)-2-(4-pentenyl)alanine.

FIG. 8 shows the capacity of stapled peptide degron chimeras comprised of a stapled peptide linked to a thalidomide degron to retain binding to recombinant cereblon, as monitored by a competitive fluorescence polarization assay. Lenalidomide serves as the positive control for the experiment. Sequences: #QLTAARLKXLGDXLHQRTBWR% (SEQ ID NO: 11); #AELEVESATQLRXFGDXLNFRQKLL% (SEQ ID NO: 12); and #RRFFGIXLTNXLKTEEGN% (SEQ ID NO: 3), wherein X is (S)-2-(4-pentenyl)alanine and # and % are as described in the Figure. “N-term Ac”=N-terminus acetylated; Lys(ivDde)=N-α-Fmoc-N-ε-1-(4,4-dimethyl-2,6-dioxocyclohex-1-ylidene)-3-methylbutyl-L-lysine; “Lys-degron”=structure depicted in FIG. 3; “N-term degron Ahx”=structure depicted in FIG. 4.

FIGS. 9A-9I show that stapled peptide degron chimeras can enter cells to compete with dBET6 for interaction with cereblon and inhibit the induced degradation of GFP-BRD4. Sequences: #RRFFGIXLTNXLKTEEGN% (SEQ ID NO:3); #FSSNRXKILXRTQILNQEWKQRRIQPV% (SEQ ID NO:2); #NLWAAQRYGRELRXBSDXFVDSFKK% (SEQ ID NO:10); #LSQEQLEHRERSLXTLRXIQRBLF% (SEQ ID NO:5); and #NLWAAQRYGRELRXBDDXFVDSFKK% (SEQ ID NO:9), wherein X is (S)-2-(4-pentenyl)alanine, and # and % are as described in the Figure. “N-term Ac”=N-terminus acetylated; Lys(ivDde)=N-α-Fmoc-N-ε-1-(4,4-dimethyl-2,6-dioxocyclohex-1-ylidene)-3-methylbutyl-L-lysine; “Lys-degron”=structure depicted in FIG. 3; “N-term degron Ahx”=structure depicted in FIG. 4.

FIG. 10 shows that the BIM-C-terminal degron, when added to an A375P melanoma cell line at 10 μM concentration induces the degradation of MCL-1. BIM-C-terminal degron is #IWIAQELRXIGDXFNAYYARR% (SEQ ID NO: 134; #=N-term Ac; %=Lys-degron (see FIG. 3 for the structure of Lys-degron)).

FIG. 11 shows that SJSA-1 cells treated with a panel of stapled peptide degron (1 μM) chimeras comprised of an MDM2/MDMX targeting stapled peptide (“ATSP”) and the thalidomide degron moiety demonstrate lower MDM2 levels in the cancer cell compared to cells treated with ATSP-7041 alone, as assessed by anti-MDM2 western analysis. Actin represents the loading control. Sequences: LTF8EYWAQLX%AA (SEQ ID NO:130) and LTF8EYWAQ#XSAA (SEQ ID NO:6). Linker1, 2, 3, and 5 are as depicted in FIG. 6.

FIG. 12 shows that treatment of SJSA-1 cells with a panel of stapled peptide degron chimeras comprised of an MDM2/MDMX targeting stapled peptide (“ATSP”), distinct linkers, and the thalidomide degron variably impair the cell viability of the cancer cells, with “5-L5” (LTF8EYWAQLX%AA (SEQ ID NO:130), where % is DAB/LINKER5/THAL) showing the most potent cytotoxic activity (left). Certain compositions bearing different linkers show no cellular activity (right). Linker1-5 are as depicted in FIG. 5.

FIG. 13 shows how genetic modification of MDM2 p60 isoform with sequence derived from Trib1 results in Cop1-mediated degradation using the expression of Myc-tagged MDM2 p60 chimera constructs in 293T cells. The native peptide sequence GFDVPD (SEQ ID NO: 26) within MDM2 is replaced by the sequences indicated (lane 3: SEQ ID NO:27; lane 4: SEQ ID NO:28; lane 5: SEQ ID NO:29; and lane 6: SEQ ID NO:30). Replacement of the native sequence with the mutant sequence DQIVPD (SEQ ID NO:30) causes destruction of the p60 chimera protein by the Cop1 protein. Lower panel: Cop1 loading control.

FIG. 14 shows binding of Myc-tagged MDM2 p60 mutant constructs to Cop1, as assessed by co-immunoprecipitation from 293T cells. Sequences: GFDVPD (SEQ ID NO:26); GFDAAD (SEQ ID NO:27); GNDVPD (SEQ ID NO:28); PQTVPD (SEQ ID NO:29); and DQIVPD (SEQ ID NO:30).

FIG. 15 provides the SAH+Trib peptide sequences that were made to evaluate the activities of targeted Trib-degron mediated protein degradation; the stapled peptide chimera sequences are assigned SEQ ID NOs.: 119-126.

FIG. 16 shows the structure of peptide degrons modeled after the TRIB sequence and the relevant moieties incorporated therein for coupling to amines or acids.

FIG. 17 shows that treatment of SJSA-1 or SJSA-X cells with a panel of stapled peptide degron chimeras comprised of an MDM2/MDMX targeting stapled peptide (e.g. an ATSP-7041-like stapled p53 peptide) and the TRIB degron moiety (20 μM) manifest variably reduced MDM2 levels in the cancer cells compared to those treated with ATSP-7041 alone (1 μM), as assessed by anti-MDM2 western analysis. Actin represents the loading control. Sequences: LTF8EYWAQ#XSAA (SEQ ID NO:6) and LTF8%YWAQLXSAA (SEQ ID NO:129).

FIG. 18 shows that treatment of SJSA-1 or SJSA-X cells with a panel of stapled peptide degron chimeras comprised of an MDM2/MDMX targeting stapled peptide (e.g. an ATSP-7041-like stapled p53 peptide) and the TRIB degron variably impairs the cell viability of the cancer cells. Sequence: LTF8%YWAQLXSAA (SEQ ID NO:129).

FIG. 19 shows that treatment of SJSA-1 or SJSA-X cells with a panel of stapled peptide degron chimeras comprised of an MDM2/MDMX targeting stapled peptide (e.g. an ATSP-7041-like stapled p53 peptide) and the VHL degron moiety (1 μM) manifest variably reduced MDM2 levels in the cancer cells compared to those treated with ATSP-7041 alone, as assessed by anti-MDM2 western analysis. Actin represents the loading control. Sequences: LTF8EYWAQ#XSAA (SEQ ID NO:6) and LTF8EYWAQLX%AA (SEQ ID NO:130).

FIG. 20 shows that treatment of SJSA-1 or SJSA-X cells with a panel of stapled peptide degron chimeras comprised of an MDM2/MDMX targeting stapled peptide (e.g. an ATSP-7041-like stapled p53 peptide) and the VHL degron variably impairs the cell viability of the cancer cells. Sequences: %TF8EYWAQLXSAA (SEQ ID NO:131) and LTF8EYWAQLX%AA (SEQ ID NO:130).

FIG. 21 shows structures of exemplary stapled peptide degron chimeras comprising one stapled peptide and a second stapled peptide. One stapled peptide targets a protein of interest (e.g., a disease-related protein of interest) and a second stapled peptide binds to a degrader protein (e.g., the stapled peptide ATSP-7041 (SEQ ID NO:6 for binding to MDM2). Also depicted is an example of a stapled peptide degron chimera comprising two copies of the same stapled peptide, wherein the stapled peptide targets a protein of interest (e.g., a disease-related protein of interest) that is a degrader protein, such that induced protein dimerization and auto-degradation can ensue upon binding of the stapled peptide degron chimera to the target protein. In each depicted example, the two stapled peptides are connected by a linker of variable length (see, e.g., FIG. 6 for exemplary linkers). Left column (from top to bottom): SEQ ID NO: 1-5, 132, 7-10, 133, and 12. Right column: SEQ ID NO:134.

FIG. 22 shows that incubation of ubiquitination machinery, including E1, E2, and recombinant MDM2, with recombinant MCL-1 and a stapled peptide degron chimera that binds to MDM2 and MCL-1, induces the ubiquitination of MCL-1 (amino acids 1-327) by MDM2.

FIG. 23 shows the structure of a stapled peptide degron chimera. The stapled peptide (ATSP-7041 (LTF8EYWAQ#XSAA (SEQ ID NO:6)) is incorporated to bind and recruit a degrader protein (MDM2) and the small molecule (JQ1) is included to bind to a disease-related protein (BRD4).

FIG. 24 shows that incubation of the ubiquitination machinery, including E1, E2, and MDM2, with recombinant BRD4 species (e.g., amino acids 342-460; amino acids 49-170) and a stapled peptide degron chimera that binds to MDM2 (e.g., stapled p53 peptide ATSP-7041) and BRD4 (e.g. small molecule JQ1), wherein the linker was composed of two beta-alanine amino acids, can induce the ubiquitination of BRD4 in two distinct regions by MDM2.

FIG. 25 shows that treatment of U2OS cells with a stapled peptide degron chimera that incorporates a stapled peptide for binding to MDM2 and a small molecule, such as JQ1, for binding to BRD4, with a linker composed of two beta-alanine amino acids as shown in FIG. 23, results in time-dependent degradation of native BRD4. Actin represents the loading control.

FIG. 26 top panel shows the chemical structures of exemplary unnatural amino acids used to generate various kinds of staples for insertion into peptides.

FIG. 26 middle panel illustrates peptides with staples of various lengths.

FIG. 26 bottom panel illustrates a staple walk along a peptide sequence.

FIG. 27 is a schematic showing representations of various kinds of double and triple stapling strategies along with exemplary staple walks for generating stapled peptides.

FIG. 28 is a schematic showing exemplary staple walks using various lengths of branched double staple moieties for generating stapled peptides.

FIG. 29 is a schematic showing exemplary chemical alterations that are employed to generate stapled peptides.

### DETAILED DESCRIPTION

This disclosure features stabilized peptide degron chimeras that act as protein degradation inducing moieties, either by combining a stabilized peptide that targets a disease-related protein with the cereblon-binding small molecule thalidomide as the “degron”, as an example, or more generally, an alternative small molecule degron, or a polypeptide sequence “degron,” including a stabilized polypeptide sequence “degron”. Stabilized peptide degron chimeras also include combining a stabilized peptide that binds and recruits a degrader protein with a small molecule or peptide, which is incorporated for targeting a disease-related protein. By combining the capacity of stabilized peptides to effectively target a broad range of intracellular proteins, previously inaccessible to small molecules, with a small molecule or peptide degron moiety capable of recruiting degrader proteins that degrade bound proteins, or by combining stabilized peptides that effectively bind and recruit a degrader protein with a small molecule or peptide that targets a disease-related protein, this new class of stapled peptide degron chimeras expands the potency and breadth of biological activity of stapled peptides. This disclosure also relates to methods for the targeted degradation of endogenous proteins through the use of stapled peptide degron chimeras that can be utilized in the treatment of disorders (e.g., proliferative disorders) caused by the presence of disease-related proteins. The present application also provides methods for making compounds of the application and intermediates thereof.

### Stabilized Peptides

A peptide helix is an important mediator of key protein-protein interactions that regulate many important biological processes (e.g., apoptosis); however, when such a helix is taken out of its context within a protein and prepared in isolation, it can unfold and adopt a random coil conformation, leading to a drastic reduction in biological activity and thus diminished therapeutic potential. To avoid this problem, one can employ structurally stabilized peptides. In some cases, structurally stabilized peptides comprise at least two modified amino acids joined by an internal (intramolecular) cross-link (or staple). Stabilized peptides as described herein include stapled peptides, stitched peptides, peptides containing multiple stitches, peptides containing multiple staples, or peptides containing a mix of staples and stitches, as well as peptides structurally reinforced by other chemical strategies (see. e.g., Balaram P. Cur. Opin. Struct. Biol. 1992; 2:845; Kemp D S, et al., J. Am. Chem. Soc. 1996; 118:4240; Orner B P, et al., J. Am. Chem. Soc. 2001; 123:5382; Chin J W, et al., Int. Ed. 2001; 40:3806; Chapman R N, et al., J. Am. Chem. Soc. 2004; 126:12252; Home W S, et al., Chem., Int. Ed. 2008; 47:2853; Madden et al., Chem Commun (Camb). 2009 Oct. 7; (37): 5588-5590; Lau et al., Chem. Soc. Rev., 2015, 44:91-102; and Gunnoo et al., Org. Biomol. Chem., 2016, 14:8002-8013; all of which are incorporated by reference herein in their entirety).

In certain embodiments, polypeptides can be stabilized by peptide stapling (see, e.g., Walensky, J Med. Chem., 57:6275-6288 (2014), the contents of which are incorporated by reference herein in its entirety). A peptide is “stabilized” in that it maintains its native secondary structure. For example, stapling allows a polypeptide, predisposed to have an α-helical secondary structure, to maintain its native α-helical conformation. This secondary structure increases resistance of the polypeptide to proteolytic cleavage and heat, and also may increase target binding affinity, hydrophobicity, and cell permeability. Accordingly, the stapled (cross-linked) polypeptides described herein have improved biological activity relative to a corresponding non-stapled (un-cross-linked) polypeptide.

“Peptide stapling” is a term coined from a synthetic methodology wherein two olefin-containing side-chains (e.g., cross-linkable side chains) present in a polypeptide chain are covalently joined (e.g., “stapled together”) using a ring-closing metathesis (RCM) reaction to form a cross-linked ring (see, e.g., Blackwell et al., J Org. Chem., 66: 5291-5302, 2001; Angew et al., Chem. Int. Ed. 37:3281, 1994). As used herein, the term “peptide stapling” includes the joining of two (e.g., at least one pair of) double bond-containing side-chains, triple bond-containing side-chains, or double bond-containing and triple bond-containing side chain, which may be present in a polypeptide chain, using any number of reaction conditions and/or catalysts to facilitate such a reaction, to provide a singly “stapled” polypeptide. The term “multiply stapled” polypeptides refers to those polypeptides containing more than one individual staple, and may contain two, three, or more independent staples of various spacing. Additionally, the term “peptide stitching,” as used herein, refers to multiple and tandem “stapling” events in a single polypeptide chain to provide a “stitched” (e.g., tandem or multiply stapled) polypeptide, in which two staples, for example, are linked to a common residue. Peptide stitching is disclosed, e.g., in WO 2008/121767 and WO 2010/068684, which are both hereby incorporated by reference in their entirety. In some instances, staples, as used herein, can retain the unsaturated bond or can be reduced.

In certain embodiments, polypeptides can be stabilized by, e.g., hydrocarbon stapling. In certain instances, the stapled peptide includes at least two (e.g., 2, 3, 4, 5, 6) amino acid substitutions, wherein the substituted amino acids are separated by two, three, or six amino acids, and wherein the substituted amino acids are non-natural amino acids with olefinic side chains. There are many known non-natural or unnatural amino acids any of which may be included in the stapled peptides. Some examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and/para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C═O)C₆H₅; —CF₃; —CN; -halo; —NO₂; CH₃), and statine. Additionally, amino acids can be derivatized to include amino acid residues that are hydroxylated, phosphorylated, sulfonated, acylated, or glycosylated.

Hydrocarbon stapled polypeptides include one or more tethers (linkages) between two non-natural amino acids, which tether significantly enhances the α-helical secondary structure of the polypeptide. Generally, the tether extends across the length of one or two helical turns (i.e., about 3.4 or about 7 amino acids). Accordingly, amino acids positioned at i and i+3; i and i+4; or i and i+7 are ideal candidates for chemical modification and cross-linking. Thus, for example, where a peptide has the sequence . . . X1, X2, X3, X4, X5, X6, X7, X8, X9 . . . , cross-links between X1 and X4, or between X1 and X5, or between X1 and X8 are useful hydrocarbon stapled forms of that peptide, as are cross-links between X2 and X5, or between X2 and X6, or between X2 and X9, etc. The use of multiple cross-links (e.g., 2, 3, 4, or more) is also contemplated. The use of multiple cross-links is very effective at stabilizing and optimizing the peptide, especially with increasing peptide length. Thus, the disclosure encompasses the incorporation of more than one cross-link within the polypeptide sequence to either further stabilize the sequence or facilitate the structural stabilization, proteolytic resistance, acid stability, thermal stability, cellular permeability, and/or biological activity enhancement of longer polypeptide stretches. Additional description regarding making and use of hydrocarbon stapled polypeptides can be found, e.g., in U.S. Patent Publication Nos. 2012/0172285, 2010/0286057, and 2005/0250680, the contents of all of which are incorporated by reference herein in their entireties.

In certain embodiments when a staple is at the i and i+3 residues, R-propenylalanine and S-pentenylalanine; or R-pentenylalanine and S-pentenylalanine are substituted for the amino acids at those positions. In certain embodiments when a staple is at the i and i+4 residues, S-pentenyl alanine is substituted for the amino acids at those positions. In certain embodiments when a staple is at the i and i+7 residues, S-pentenyl alanine and R-octenyl alanine are substituted for the amino acids at those positions. In some instances, when the peptide is stitched, the amino acids of the peptide to be involved in the “stitch” are substituted with Bis-pentenylglycine, S-pentenylalanine, and R-octenylalanine; or Bis-pentenylglycine, S-octenylalanine, and R-octenylalanine.

Staple or stitch positions can be varied by testing different staple locations in a staple walk.

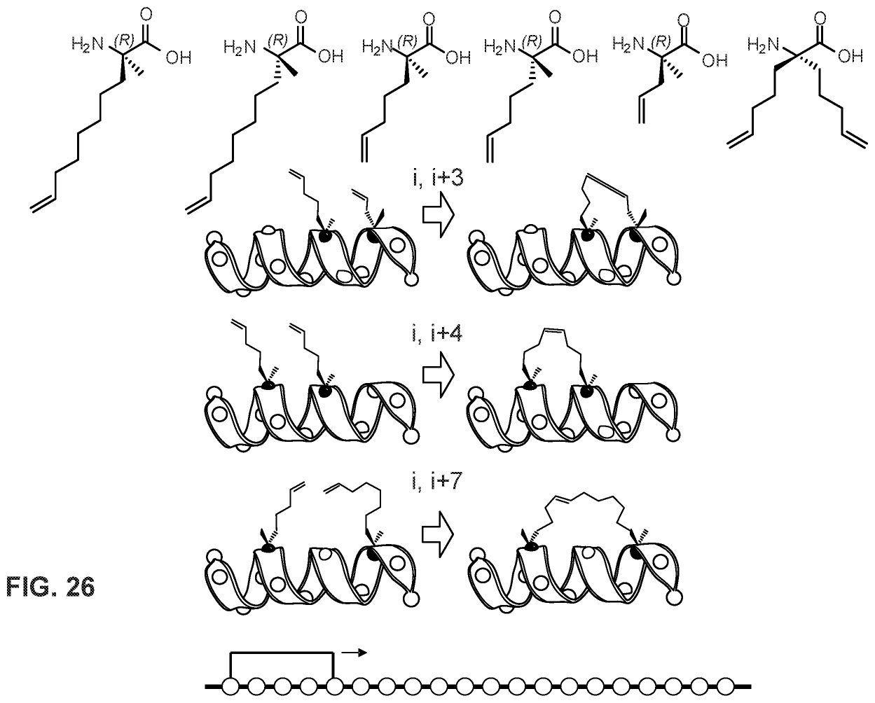

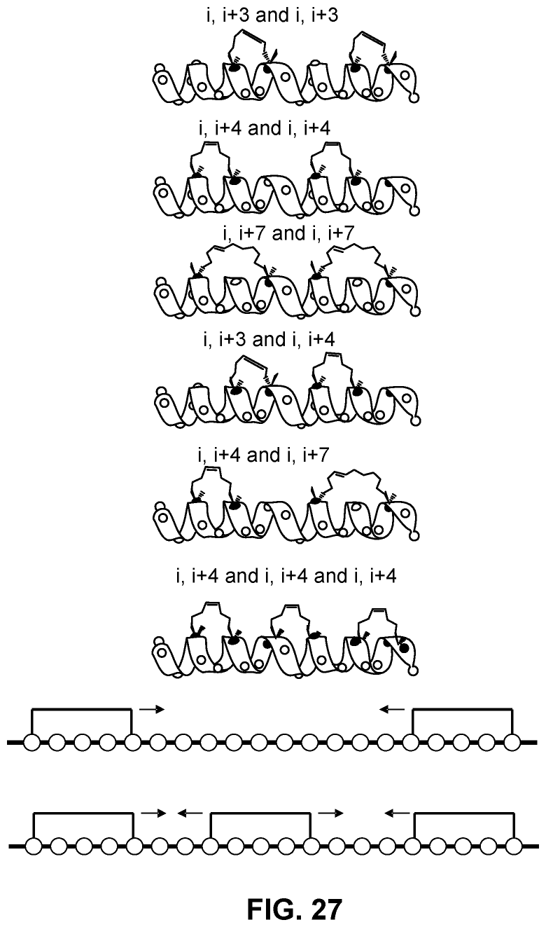

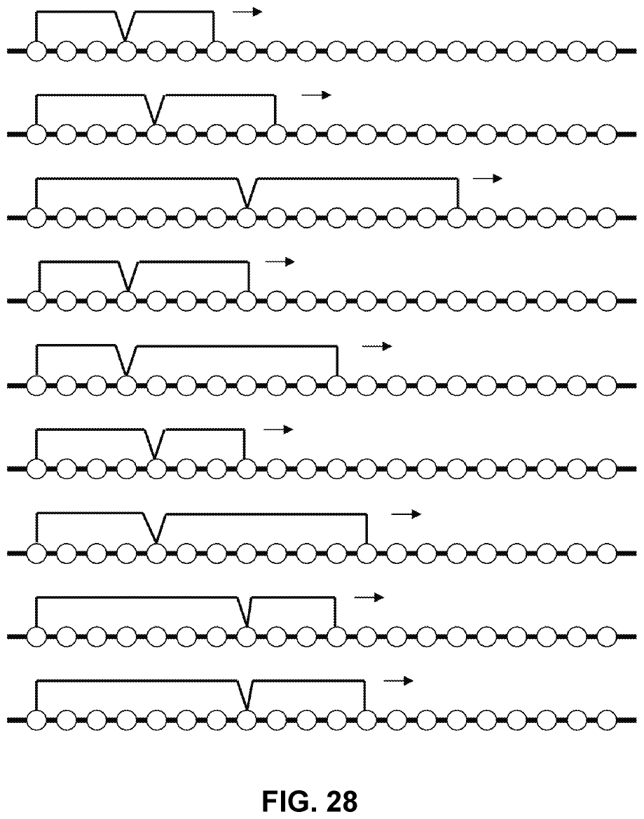

FIG. 26 (top) shows exemplary chemical structures of non-natural amino acids that can be used to generate various crosslinked compounds. FIG. 26 (middle) illustrates peptides with hydrocarbon cross-links between positions i and i+3; i and i+4; and i and i+7 residues. FIG. 26 (bottom) illustrates a staple walk along a peptide sequence. FIG. 27 shows various peptide sequences with double and triple stapling strategies, and exemplary staple walks. FIG. 28 illustrates exemplary staple walks using various lengths of branched stitched moieties.

In one aspect, a stabilized polypeptide has the formula (I),

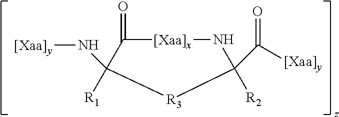

wherein:
each R₁ and R₂ are independently H or a C₁ to C₁₀ alkyl, alkenyl, alkynyl, arylalkyl, cycloalkylalkyl, heteroarylalkyl, or heterocyclylalkyl;
R₃ is alkyl, alkenyl, alkynyl; [R₄—K—R₄]ₙ; each of which is substituted with 0-6 R₅;
R₄ is alkyl, alkenyl, or alkynyl;
R₅ is halo, alkyl, OR₆, N(R₆)₂, SR₆, SOR₆, SO₂R₆, CO₂R₆, R₆, a fluorescent moiety, or a radioisotope;
K is O, S, SO, SO₂, CO, CO₂, CONR₆, or

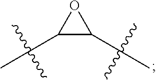 [SMILES: *C1OC1*]

R₆ is H, alkyl, or a therapeutic agent;
n is an integer from 1-4;
x is an integer from 2-10;
each y is independently an integer from 0-100;
z is an integer from 1-10 (e.g., 1, 2, 3, 4, 5, 6, 7, 8, 9, 10);
and each Xaa is independently an amino acid.

The tether can include an alkyl, alkenyl, or alkynyl moiety (e.g., C₅, C₈, or C₁₁ alkyl, a C₅, C₈, or C₁₁ alkenyl, or C₅, C₈, or C₁₁ alkynyl). The tethered amino acid can be alpha disubstituted (e.g., C₁-C₃ or methyl).

In some instances, x is 2, 3, or 6. In some instances, each y is independently an integer between 1 and 15, or 3 and 15. In some instances, R₁ and R₂ are each independently H or C₁-C₆ alkyl. In some instances, R₁ and R₂ are each independently C₁-C₃ alkyl. In some instances, at least one of R₁ and R₂ are methyl. For example, R₁ and R₂ can both be methyl. In some instances, R₃ is alkyl (e.g., C₈ alkyl) and x is 3. In some instances, R₃ is C₁₁ alkyl and x is 6. In some instances, R₃ is alkenyl (e.g., C₈ alkenyl) and x is 3. In some instances, x is 6 and R₃ is C₁₁ alkenyl. In some instances, R₃ is a straight chain alkyl, alkenyl, or alkynyl. In some instances, R₃ is —CH₂—CH₂—CH₂—CH═CH—CH₂—CH₂—CH₂—.

In another aspect, the two alpha, alpha disubstituted stereocenters are both in the R configuration or S configuration (e.g., i, i+4 cross-link), or one stereocenter is R and the other is S (e.g., i, i+7 cross-link). Thus, where formula I is depicted as:

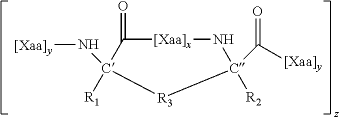

the C′ and C″ disubstituted stereocenters can both be in the R configuration or they can both be in the S configuration, e.g., when x is 3. When x is 6, the C′ disubstituted stereocenter is in the R configuration and the C″ disubstituted stereocenter is in the S configuration. The R₃ double bond can be in the E or Z stereochemical configuration.

In some instances, R₃ is [R₄—K—R₄]ₙ; and R₄ is a straight chain alkyl, alkenyl, or alkynyl.

In some embodiments, the disclosure features internally cross-linked (“stapled” or “stitched”) peptides, wherein the side chains of two amino acids separated by two, three, or six amino acids are replaced by an internal staple; the side chains of three amino acids are replaced by an internal stitch; the side chains of four amino acids are replaced by two internal staples, or the side chains of five amino acids are replaced by the combination of an internal staple and an internal stitch. The stapled/stitched peptide can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length.

In certain instances, the stabilized peptide is a peptide of an intracellular protein. In certain instances, the stabilized peptide is a peptide of a disease causing or disease-related protein. In certain instances, the stabilized peptide is a peptide of a bacterial protein. In certain instances, the stabilized peptide is a peptide of a human protein. In certain instances, the stabilized peptide is a peptide of an oncogenic protein. Non-limiting examples of oncogenic proteins include BCL2, BCLXL, MCL-1, BFL-1, BCL-w, BCL-B, EZH2, HDM2/HDMX, KRAS/NRAS/HRAS, MYC, 0-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5.

Non-limiting examples of stapled peptides are listed below:

PUMA
(SEQ ID NO: 1)
QWAREIGAQLRX₁BADX₂LNAQYERR
EZH2
(SEQ ID NO: 2)
FSSNRX₁KILX₂RTQILNQEWKQRRIQPV
SOS
(SEQ ID NO: 3)
RRFFGIX₁LTNX₂LKTEEGN
MCL-1
(SEQ ID NO: 4)
RKALETLRRVGDGVX₁RNHX₂TAF
BCL9
(SEQ ID NO: 5)
LSQEQLEHRERSLX₁TLRX₂IQRBLF
p53
(SEQ ID NO: 6)
LTF8EYWAQ#XSAA
BID
(SEQ ID NO: 7)
DIIRNIARHLAX₁VGDX₂BDRSI
BIM
(SEQ ID NO: 8)
IWIAQELRX₁IGDX₂FNAYYARR
BAD S153D
(SEQ ID NO: 9)
NLWAAQRYGRELRX₁BDDX₂FVDSFKK
BAD
(SEQ ID NO: 10)
NLWAAQRYGRELRX₁BSDX₂FVDSFKK
HRK
(SEQ ID NO: 11)
QLTAARLKX₁LGDX₂LHQRTBWR
NOXA
(SEQ ID NO: 12)
AELEVESATQLRX₁FGDX₂LNFRQKLL
PUMA
(SEQ ID NO: 13)
QWAREIGAQLRX₁BADX₂LNAQYERR
EZH2
(SEQ ID NO: 14)
FSSNRX₁KILX₂RTQILNQEWKQRRIQPV
SOS
(SEQ ID NO: 15)
RRFFGI X₁LTNX₂LKTEEGN
MCL-1
(SEQ ID NO: 16)
RKALETLRRVGDGVX₁RNHX₂TAF
BCL9
(SEQ ID NO: 17)
LSQEQLEHRERSLX₁TLRX₂IQRBLF
p53
(SEQ ID NO: 18)
LTF8EYWAQ#XSAA
BID
(SEQ ID NO: 19)
DIIRNIARHLAVVGDX₂BDRSI
BIM
(SEQ ID NO: 20)
IWIAQELRVIGDX₂FNAYYARR
BAD-S153D
(SEQ ID NO: 21)
NLWAAQRYGRELRX₁BDDX₂FVDSFKK
BAD
(SEQ ID NO: 22)
NLWAAQRYGRELRX₁BSDX₂FVDSFKK
HRK
(SEQ ID NO: 23)
QLTAARLKX₁LGDX₂LHQRTBWR
NOXA
(SEQ ID NO: 24)
AELEVESATQLRX₁FGDX₂LNFRQKLL
p53 (ATSP-7041 #26L)
(SEQ ID NO: 134)
LTF8EYWAQLXSAA,

wherein, 8=R-octenyl alanine; B=norleucine; #=cyclobutylalanine; X=S-pentenyl alanine, and, in some instances, X₁ and X2 are the same (e.g., S-penetenyl alanine).

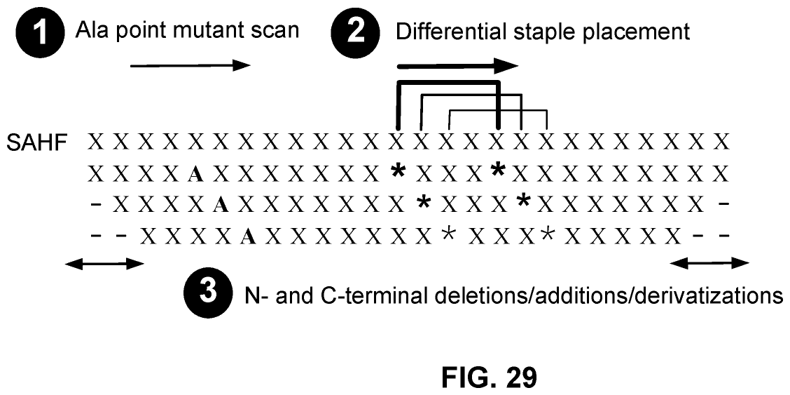

In certain embodiments, the stapled polypeptide comprises or consists of the amino acid sequence set forth in any one of SEQ ID NOs:1 to 24 and 134. In certain embodiments, this disclosure features stabilized peptides that differ from the peptides disclosed above in that they vary in the location of the staple/stitch. In certain embodiments, this disclosure features stabilized peptides that differ from the peptides disclosed above in that they vary from the above-disclosed sequences in having 1 to 7 (e.g., 1, 2, 3, 4, 5, 6, 7) amino acid substitutions on the non-interacting face of the alpha-helix of these peptides. In certain instances, the substitutions are conservative. In other instances, the substitutions are non-conservative. In certain embodiments, this disclosure features stabilized peptides that differ from the peptides disclosed above in that they vary from the above-disclosed sequences in having 1 to 5 (e.g., 1, 2, 3, 4, 5) amino acid substitutions on the interacting face of the alpha-helix of these peptides. In certain instances, the substitutions are conservative. Exemplary types of variations/modifications to stapled peptides are illustrated in FIG. 29.

In certain embodiments, the stapled peptide is not a Bcl-2 homology 3 (BH3) domain polypeptide (e.g., not a BH3 domain from MCL-1, not an MCL-1 Stabilized Alpha Helix of BCL2 domain (SAHB), or not MCL-1 SAHBD).

In certain embodiments, the stabilized peptide (e.g., stapled peptide) directly binds to and recruits a degrader protein, such as the ubiquitin E3 ligase MDM2. For example, the E3 ligase MDM2 can be potently bound by stapled p53 peptides known in the art and incorporated by reference herein in their entireties. In certain instances, the peptide degron is a stabilized or stapled peptide that directly binds to and recruits a complex that contains a degrader protein, such as the complex between MDMX and the ubiquitin E3 ligase MDM2. In this example, a stapled p53 peptide can potently bind MDMX and recruit the MDMX/MDM2 complex such that MDM2 can be recruited as a degrader protein.

In certain embodiments, the stabilized peptide binds, directly or indirectly, to a degrader protein, such as, e.g., an E3 ubiquitin ligase or a substrate adaptor for an E3 ubiquitin ligase. In certain embodiments, the stabilized peptide binds, directly or indirectly, to an E3 ubiquitin ligase. In some embodiments, the stabilized peptide binds, directly or indirectly, to E3 ligase (e.g., MDM2) or a protein that is complexed to an E3 ligase, such as MDMX binding to MDM2. In certain embodiments, the E3 ubiquitin ligase is a RING E3 ubiquitin ligase (e.g., Mdm2-MdmX, TRIM5α, c-CBL, cIAP, RNF4, BIRC7, IDOL, BRCA1-BARD1, RINGB-Bmi1, E4B, CHIP, Prp19). In certain embodiments, the E3 ubiquitin ligase is a HECT E3 ubiquitin ligase (e.g., Smurf1, Smurf2, Itch, E6AP). In certain embodiments, the E3 ubiquitin ligase is a RBR E3 ubiquitin ligase (e.g., Parkin, Parc, RNF144 (A/B), HOIP, HHARI). See, e.g., Morreale and Walden, Cell 165, 2016 DOI http./dx.doi.org/10.1016/j.cell.2016.03.003 for non-limiting examples of E3 ubiquitin ligases.

Non-limiting examples of other stabilized peptides that can be employed in the chimeric fusions described herein are provided in U.S. Pat. Nos. 9,834,581; 9,822,165; 9,695,224; 9,617,309; 9,579,395; 9,556,229; 9,556,227; 9,527,896; 9,522,947; 9,517,252; 9,505,816; 9,505,804; 9,505,801; 9,493,510; 9,464,125; 9,485,202; 9,458,189; 9,416,162; 9,408,885; 9,346,868; 9,296,805; 9,227,995; 9,175,047; 9,175,045; 9,163,330; 9,096,684; 9,079,970; 8,957,026; 8,937,154; 8,933,109; 8,927,500; 8,889,632; 8,592,377; 8,586,707; 8,324,153; and U.S. Patent Application Publication Nos. 20170247423; 20170240604; 20170212125; 20170165320; 20170066747; 20170015716; 20160376336; and 20160244494, the contents of all of which are incorporated by reference in their entirety herein (especially the disclosure of stabilized (e.g., stapled or stitched) peptides).

While hydrocarbon tethers are common, other tethers can also be employed in the stabilized peptides described herein. For example, the tether can include one or more of an ether, thioether, ester, amine, or amide, or triazole moiety. In some cases, a naturally occurring amino acid side chain can be incorporated into the tether. For example, a tether can be coupled with a functional group such as the hydroxyl in serine, the thiol in cysteine, the primary amiein lysine, the acid in aspartate or glutamate, or the amide in asparagine or glutamine. Accordingly, it is possible to create a tether using naturally occurring amino acids rather than using a tether that is made by coupling two non-naturally occurring amino acids. It is also possible to use a single non-naturally occurring amino acid together with a naturally occurring amino acid. Triazole-containing (e.g., 1, 4 triazole or 1, 5 triazole) crosslinks can be used (see, e.g., Kawamoto et al. 2012 Journal of Medicinal Chemistry 55:1137; WO 2010/060112). In addition, other methods of performing different types of stapling are well known in the art and can be employed (see, e.g., Lactam stapling: Shepherd et al., J. Am. Chem. Soc., 127:2974-2983 (2005); UV-cycloaddition stapling: Madden et al., Bioorg. Med. Chem. Lett., 21:1472-1475 (2011); Disulfide stapling: Jackson et al., Am. Chem. Soc., 113:9391-9392 (1991); Oxime stapling: Haney et al., Chem. Commun., 47:10915-10917 (2011); Thioether stapling: Brunel and Dawson, Chem. Commun., 552-2554 (2005); Photoswitchable stapling: J. R. Kumita et al., Proc. Natl. Acad. Sci. U.S.A, 97:3803-3808 (2000); Double-click stapling: Lau et al., Chem. Sci., 5:1804-1809 (2014); Bis-lactam stapling: J. C. Phelan et al., J Am. Chem. Soc., 119:455-460 (1997); and Bis-arylation stapling: A. M. Spokoyny et al., J Am. Chem. Soc., 135:5946-5949 (2013)).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers spanning from amino acids i to i+3, i to i+4, and i to i+7 are common in order to provide a tether that is primarily on a single face of the alpha helix, the tethers can be synthesized to span any combinations of numbers of amino acids and also used in combination to install multiple tethers.

In some instances, the hydrocarbon tethers (i.e., cross links) described herein can be further manipulated. In one instance, a double bond of a hydrocarbon alkenyl tether, (e.g., as synthesized using a ruthenium-catalyzed ring closing metathesis (RCM)) can be oxidized (e.g., via epoxidation, aminohydroxylation or dihydroxylation) to provide one of compounds below.

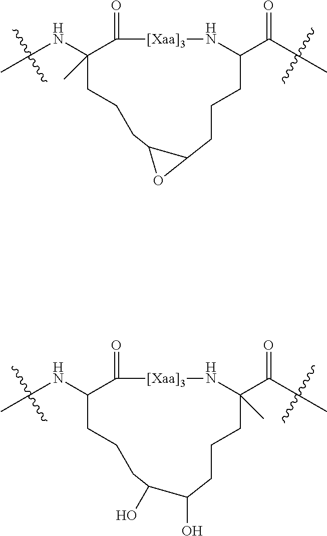

Either the epoxide moiety or one of the free hydroxyl moieties can be further functionalized. For example, the epoxide can be treated with a nucleophile, which provides additional functionality that can be used, for example, to attach a therapeutic agent. Such derivatization can alternatively be achieved by synthetic manipulation of the amino or carboxy-terminus of the polypeptide or via the amino acid side chain. Other agents can be attached to the functionalized tether, e.g., an agent that facilitates entry of the polypeptide into cells.

In some instances, alpha disubstituted amino acids are used in the polypeptide to improve the stability of the alpha helical secondary structure. However, alpha disubstituted amino acids are not required, and instances using mono-alpha substituents (e.g., in the tethered amino acids) are also envisioned.

The stapled polypeptides can include a drug, a toxin, a derivative of polyethylene glycol; a second polypeptide; a carbohydrate, etc. Where a polymer or other agent is linked to the stapled polypeptide is can be desirable for the composition to be substantially homogeneous.

The addition of polyethelene glycol (PEG) molecules can improve the pharmacokinetic and pharmacodynamic properties of the polypeptide. For example, PEGylation can reduce renal clearance and can result in a more stable plasma concentration. PEG is a water soluble polymer and can be represented as linked to the polypeptide as formula:

XO—(CH₂CH₂O)ₙ—CH₂CH₂—Y where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the polypeptide. Y may also be a maleimide linkage to a thiol group (including but not limited to, the thiol group of cysteine). Other methods for linking PEG to a polypeptide, directly or indirectly, are known to those of ordinary skill in the art. The PEG can be linear or branched. Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described in WO 99/34833; WO 99/14259, and U.S. Pat. No. 6,348,558.

In certain embodiments, macromolecular polymer (e.g., PEG) is attached to an agent described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH₂)ₙC(O)—, wherein n=2-20 can be used. These alkyl linkers may further be substituted by any non-sterically hindering group such as lower alkyl (e.g., C₁-C₆) lower acyl, halogen (e.g., C₁, Br), CN, NH₂, phenyl, etc. U.S. Pat. No. 5,446,090 describes a bifunctional PEG linker and its use in forming conjugates having a peptide at each of the PEG linker termini.

The stabilized peptides can also be modified, e.g., to further facilitate cellular uptake or increase in vivo stability, in some embodiments. For example, acylating or PEGylating a peptidomimetic macrocycle facilitates cellular uptake, increases bioavailability, increases blood circulation, alters pharmacokinetics, decreases immunogenicity and/or decreases the needed frequency of administration.

In some embodiments, the stapled peptides disclosed herein have an enhanced ability to penetrate cell membranes (e.g., relative to non-stapled peptides).

Methods of synthesizing the stabilized peptides described herein are known in the art. Nevertheless, the following exemplary method may be used. It will be appreciated that the various steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The stabilized peptides can be made by chemical synthesis methods, which are well known to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH2 protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from Advanced Chemtech. Peptide bonds can be replaced, e.g., to increase physiological stability of the peptide, by: a retro-inverso bonds (C(O)—NH); a reduced amide bond (NH—CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF=CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

The polypeptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. As indicated above, peptides can be conjugated to, for example, polyethylene glycol (PEG); alkyl groups (e.g., C₁-C₂₀ straight or branched alkyl groups); fatty acid radicals; and combinations thereof. α, α-Disubstituted non-natural amino acids containing olefinic side chains of varying length can be synthesized by known methods (Williams et al. J Am. Chem. Soc., 113:9276, 1991; Schafmeister et al., J Am. Chem Soc., 122:5891, 2000; and Bird et al., Methods Enzymol., 446:369, 2008; Bird et al, Current Protocols in Chemical Biology, 2011). For peptides where an i linked to i+7 staple is used (two turns of the helix stabilized) either: a) one S5 amino acid and one R₈ is used; or b) one S8 amino acid and one R₅ amino acid is used. R₈ is synthesized using the same route, except that the starting chiral auxillary confers the R-alkyl-stereoisomer. Also, 8-iodooctene is used in place of 5-iodopentene. Inhibitors are synthesized on a solid support using solid-phase peptide synthesis (SPPS) on MBHA resin (see, e.g., WO 2010/148335).

Fmoc-protected α-amino acids (other than the olefinic amino acids Fmoc-S₅—OH, Fmoc-R₈—OH, Fmoc-R₈—OH, Fmoc-S₈—OH and Fmoc-R₅—OH), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, Calif.). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:31, 2003).

Again, methods suitable for obtaining (e.g., synthesizing), stapling, and purifying the peptides disclosed herein are also known in the art (see, e.g., Bird et. al., Methods in Enzymol., 446:369-386 (2008); Bird et al, Current Protocols in Chemical Biology, 2011; Walensky et al., Science, 305:1466-1470 (2004); Schafmeister et al., J Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some embodiments, the peptides are substantially free of non-stapled peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific embodiment, a 50%/50% DMSO/NMP solution is used. The solution may be incubated for a period of 1, 6, 12 or 24 hours, following which the resin may be washed, for example with dichloromethane or NMP. In one embodiment, the resin is washed with NMP. Shaking and bubbling an inert gas into the solution may be performed.

Properties of the stabilized (e.g., stapled) polypeptides of the invention can be assayed, for example, using the methods described below.

Assays to Determine α-Helicity:

Compounds are dissolved in an aqueous solution (e.g. 5 mM potassium phosphate solution at pH 7, or distilled H₂O, to concentrations of 25-50 μM). Circular dichroism (CD) spectra are obtained on a spectropolarimeter (e.g., Jasco J-710, Aviv) using standard measurement parameters (e.g. temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity by the reported value for a model helical decapeptide (Yang et al., Methods Enzymol. 130:208 (1986)).

Assays to Determine Melting Temperature (Tm):

Cross-linked or the unmodified template peptides are dissolved in distilled H₂O or other buffer or solvent (e.g. at a final concentration of 50 μM) and Tm is determined by measuring the change in ellipticity over a temperature range (e.g. 4 to 95° C.) on a spectropolarimeter (e.g., Jasco J-710, Aviv) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

In Vitro Protease Resistance Assays:

The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The peptidomimetic macrocycles of the present invention may be subjected to in vitro enzymatic proteolysis (e.g. trypsin, chymotrypsin, pepsin) to assess for any change in degradation rate compared to a corresponding uncrosslinked or alternatively stapled polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln[S] versus time.

Peptidomimetic macrocycles and/or a corresponding uncrosslinked polypeptide can be each incubated with fresh mouse, rat and/or human serum (e.g. 1-2 mL) at 37° C. for, e.g., 0, 1, 2, 4, 8, and 24 hours. Samples of differing macrocycle concentration may be prepared by serial dilution with serum. To determine the level of intact compound, the following procedure may be used: The samples are extracted, for example, by transferring 100 μL of sera to 2 ml centrifuge tubes followed by the addition of 10 μL of 50% formic acid and 500 μL acetonitrile and centrifugation at 14,000 RPM for 10 min at 4+/−2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of macrocycles in serum.

In Vivo Protease Resistance Assays:

A key benefit of peptide stapling is the translation of in vitro protease resistance into markedly improved pharmacokinetics in vivo.

In vitro Binding Assays:

To assess the binding and affinity of peptidomimetic macrocycles and peptidomimetic precursors to acceptor proteins, a fluorescence polarization assay (FPA) can be used, for example. The FPA technique measures the molecular orientation and mobility using polarized light and fluorescent tracer. When excited with polarized light, fluorescent tracers (e.g., FITC) attached to molecules with high apparent molecular weights (e.g. FITC-labeled peptides bound to a large protein) emit higher levels of polarized fluorescence due to their slower rates of rotation as compared to fluorescent tracers attached to smaller molecules (e.g. FITC-labeled peptides that are free in solution).

Cellular Analyses:

Cultured cells (e.g., cancer cells) are treated with stapled peptide-degron chimeras and targeted protein levels monitored over time by western analysis. Negative control proteins, not targeted by the chimeric peptides, are likewise monitored as a demonstration of targeted degradation specificity. Depending on the specific target, phenotypic outcomes, such as apoptosis induction, are assessed by a combination of viability, annexin V binding, caspase 3/7 activation, and mitochondrial cytochrome c release assays.

### Peptide Degrons

This disclosure features peptide degrons that bind a protein that is the substrate adaptor for an ubiquitin E3 ligase. In some instances, the degrons bind a WD-40 protein that is the substrate adaptor for an ubiquitin E3 ligase. The degron binds to the substrate recognition domain of the Ubiquitin E3 ligase in a shallow groove and is tolerant of elaboration (i.e., conjugation of a stapled peptide sequence) at either the N- or C-terminus. Exemplary substrate adaptors for an ubiquitin E3 ligase include MDM2, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, and FZR1. While WD40 containing E3 ligases comprise many of the E3 ligases, there are other types of E3 ligases and degrons that bind a protein that is the substrate adaptor of such E3 ligases are also encompassed by this disclosure.

In certain instances, the peptide degrons are based on the Trib1 protein sequence: DQIVPEY (SEQ ID NO:25) or variants thereof.

| Position | 1 | 2 | 3 | 4 | 5 | 6 | 7 |
|---|---|---|---|---|---|---|---|
| Amino Acid | D | Q | I | V | P | E | Y |

The degrons of this disclosure include variants of SEQ ID NO:25, wherein the variant includes one or more (e.g., 1, 2, 3, 4, 5) amino acid substitutions; one or more deletions (e.g., 1, 2, 3); one or more insertions (e.g., 1, 2, 3); or a combination of any two or more thereof. In some instances, the variant of SEQ ID NO:25 has one or more (e.g., 1, 2, 3, 4, 5) substitutions. In certain instances one or more (e.g., 1, 2, 3, 4, 5, 6, 7) of these substitutions are not to A, R at any of positions 1 to 6 of SEQ ID NO:25. In certain instances, these substitutions do not include a substitution of V at position 4 to an I in SEQ ID NO:25. In some instances, the variant of SEQ ID NO:25 has one or more (e.g., 1, 2, 3) deletions. In some instances, the variant of SEQ ID NO:25 has one or more (e.g., 1, 2, 3) insertions. In some instances, the variant of SEQ ID NO:25 has one or more (e.g., 1, 2, 3, 4, 5) substitutions and one or more (e.g., 1, 2, 3) deletions. In some instances, the variant of SEQ ID NO:25 has one or more (e.g., 1, 2, 3, 4, 5) substitutions and one or more insertions (e.g., 1, 2, 3). In some instances, the variant of SEQ ID NO:25 has one or more (e.g., 1, 2, 3) deletions and one or more (e.g., 1, 2, 3) insertions. In some instances, the variant of SEQ ID NO:25 has one to six, one to five, one to four, one to three, two, or one amino acid substitution within SEQ ID NO:25. In certain instances, position 4(V) and/or position 5 (P) of SEQ ID NO:25 are not substituted. In some instances, one or more of positions 1(D), position 2 (Q), position 3 (I), and position 6 (E) of SEQ ID NO:25 are substituted. In some embodiments, the peptide degron comprises SEQ ID NO:25 except that any one of positions 1 through 7 are not substituted by alanine. In some embodiments, the peptide degron comprises an amino acid sequence comprising SEQ ID NO:25 except that any one of positions 1 through 7 are not substituted by arginine. In some embodiments, the peptide degron comprises SEQ ID NO:25 except that position 4 is not substituted with an isoleucine. In some instances, the variant of SEQ ID NO:25 has one deletion. The deletion may be at the C-terminus or at the N-terminus of SEQ ID NO:25.

In some instances, the peptide degron is a peptide that binds F-box/WD repeat-containing protein 7 (FBXW7) protein. In one embodiment, the peptide degron comprises the amino acid sequence phospho-Ser/phospho-ThrPXXE/phospho-Ser/phospho-Thr (pS/pT-P—Xₐ—X_{b}-E/pS/pT) (SEQ ID NO: 46), wherein Xₐ and X_{b} are independently any amino acid. In some instances Xₐ=P. In some instances, X_{b}=V, L, or Q. In other instances, Xₐ=P and X_{b}=V, L, or Q. In certain embodiments, the peptide degron is a variant of SEQ ID NO:46. Such variants include peptides that differ from SEQ ID NO:46 in having one or more (e.g., 1, 2, 3, 4) amino acid substitutions; one or more deletions (e.g., 1, 2, 3); one or more insertions (e.g., 1, 2, 3); or a combination of any two or more thereof. In some instances, the variant of SEQ ID NO:46 has one or more (e.g., 1, 2, 3, 4) substitutions. In some instances, the variant of SEQ ID NO:46 has one or more (e.g., 1, 2, 3) deletions. In some instances, the variant of SEQ ID NO:46 has one or more (e.g., 1, 2, 3) insertions. In some instances, the variant of SEQ ID NO:46 has one or more (e.g., 1, 2, 3, 4) substitutions and one or more (e.g., 1, 2, 3) deletions. In some instances, the variant of SEQ ID NO:46 has one or more (e.g., 1, 2, 3, 4) substitutions and one or more insertions (e.g., 1, 2, 3). In some instances, the variant of SEQ ID NO:46 has one or more (e.g., 1, 2, 3) deletions and one or more (e.g., 1, 2, 3) insertions. In some instances, the variant of SEQ ID NO:46 has one to five, one to four, one to three, two, or one amino acid substitution within SEQ ID NO:46. In certain instances, position 2(P) is not substituted. In certain instances, position 1 is pS and position 2 is P. In certain instances, position 1 is pS, position 2 is P, and position 5 is E. In certain instances, position 1 is pS, position 2 is P, and position 5 is pS. In certain instances, position 1 is pS, position 2 is P, and position 5 is pT. In certain instances, position 1 is pT, position 2 is P, and position 5 is E. In certain instances, position 1 is pT, position 2 is P, and position 5 is pS. In certain instances, position 1 is pT, position 2 is P, and position 5 is pT.

In certain instances, the peptide degrons are based on a natural binding consensus sequence of a peptide that binds a WD40-repeat protein that is the substrate adaptor for an E3 ubiquitin ligase. In some cases the peptide degrons are variants (e.g., substitution, deletion, or insertion variants) of the natural binding consensus sequence of a peptide that binds a WD40-repeat protein that is the substrate adaptor for an E3 ubiquitin ligase. Non-limiting examples of natural binding consensus sequence of a peptide that binds a WD40-repeat protein that is the substrate adaptor for an E3 ubiquitin ligase are provided below (the sequences are assigned SEQ ID NOs.: 65 to 92 from top to bottom):

| | Number | |
|---|---|---|
| | of known | |
| E3 ligase/degron | instances | Motif pattern^{†‡} |
| APC/C (DBOX) | 8 | .R..L..[LIVM]. |
| APC/C (KEN) | 15 | .KEN. |
| APC/C (ABBA) | 1 | [FIVL].[ILMVP][FHY].[DE].{0,3}[DEST] |
| APCC_TPR_1 | 16 | .[ILM]R$ |
| CBL (PTK) | 3 | [DN].Y[ST]..P |
| CBL (MET) | 5 | DYR |
| COP1 | 7 | [DE][DE].{2,3}VP[DE] |
| CRL4_CDT2_1 | 6 | [NQ]{0,1}..[ILMV][ST][DEN][FY][FY].{2,3}[KR]{2,3}[\hat{ }DE] |
| CRL4_CDT2_2 | 1 | [NQ]{0,1}..[ILMV]T[DEN][HMFY][FMY].{2,3}[KR]{2,3}[\hat{ }DE] |
| Kelch_KEAP1_1 | 13 | [DNS].[DES][TNS]GE |
| Kelch_KEAP1_2 | 1 | QD.DLGV |
| Kelch_actinfilin | 1 | [AP]P[MV][IM]V |
| Kelch_KLHL3 | 4 | E.EE.E[AV]DQH |
| MDM2_SWIB | 5 | F[\hat{ }P]{3}W[\hat{ }P]{2,3}[VIL] |
| Nend_Nbox_1 | 1 | \hat{ }M{0,1}[FYLIW][\hat{ }P] |
| Nend_UBRbox_1 | 2 | \hat{ }M{0,1}[RK][\hat{ }P]. |
| Nend_UBRbox_2 | 1 | \hat{ }M{0,1}([ED]). |
| Nend_UBRbox_3 | 1 | \hat{ }M{0,1}([NQ]). |
| Nend_UBRbox_4 | 8 | \hat{ }M{0,1}(C). |
| ODPH_VHL_1 | 8 | [IL]A(P).{6,8}[FLIVM].[FLIVM] |
| SCF_COI1_1 | 6 | ..[RK][RK].SL..F[FLM].[RK]R[HRK].[RK]. |
| SCF_FBW7_1 | 7 | [LIVMP].{0,2}(T)P..([ST]) |
| SCF_FBW7_2 | 2 | [LIVMP].{0,2}(T)P..E |
| SCF_SKP2-CKS1_1 | 3 | ..[DE].(T)P.K |
| SCF_TIR1_1 | 7 | .[VLIA][VLI]GWPP[VLI]...R. |
| SCF-TRCP1 | 18 | D(S)G.{2,3}([ST]) |
| SIAH | 8 | .P.A.V.P[\hat{ }P] |
| SPOP | 13 | [AVP].[ST][ST][ST] |

^{†}The motif pattern uses the following nomenclature: ‘.’ specifies any amino acid type, ‘[X]’ specifies the allowed amino acid type(s) at that position, ‘\hat{ }X’ at the beginning of the pattern specifies that the sequence starts with amino acid type X, ‘[\hat{ }X]’ denotes that the position can have any amino acid other than type X, numbers specified as the following ‘X{x,y}’, where x and y specify the minimum and maximum number of ‘X’ amino acid type required at that position. ‘$’ sign indicates the C-terminal of the protein chain. Conserved residue positions within the primary degron that are known to be post-translationally modified (for example, phosphorylation and proline hydroxylation) are shown in boldface.

Any other peptide degron known in the art can also be employed in this invention. See, e.g., Mészáros et al., Sci. Signal., 10(470):eaak9982 (2017); Guharoy et al., Nature Communications, 7:10239, doi:10.1038/ncomms10239 (2016); U.S. Pat. Nos. 9,783,575; 9,297,017; and 9,115,184, all of which are incorporated by reference herein in their entireties.

In certain instances, the peptide degron has the amino acid sequence of a peptide listed below, or a variant thereof:

E3 ligase: MDM2
(SEQ ID NO: 31)
FSDLWKLL;
E3 ligase: SKP2-CKS1
(SEQ ID NO: 32)
SVEQTPKK;
E3 ligase: (β-TrCP1
(SEQ ID NO: 32)
DSGIHS;
E3 ligase: FBXW7
(SEQ ID NO: 33)
LLPTPPLS;
E3 ligase: SPOP
(SEQ ID NO: 34)
ASSSS;
E3 ligase: VHL
(SEQ ID NO: 35)
LAPAAGDTIISLDF;
E3 ligase: FBXW7
(SEQ ID NO: 36)
PFLTPSPE;
E3 ligase: ITCH
(SEQ ID NO: 37)
PPPY;
E3 ligase: KEAP1
(SEQ ID NO: 38)
DEETGE;
E3 ligase: KEAP1
(SEQ ID NO: 39)
QDIDLGV;
E3 ligase: CBL
(SEQ ID NO: 40)
LLQPNNYQFC;
DYR-E3ligase:CBL
E3 ligase: CBL
(SEQ ID NO: 41)
RAVENQYSFY;
E3 ligase: CDH1
(SEQ ID NO: 42)
QKENS;
E3 ligase: CDC20/CDH1
(SEQ ID NO: 43)
FDIYMD;
E3 ligase: CDC20/CDH1
(SEQ ID NO: 44)
PRTALGDIG;
E3 ligase: PTTG1
(SEQ ID NO: 45)
DKENG;
E3 ligase: APC/C
(SEQ ID NO: 93)
HRKHLQEIP;
E3 ligase: APC/C
(SEQ ID NO: 94)
SKENV;
E3 ligase: APC/C
(SEQ ID NO: 95)
TRIR;
E3 ligase: COP1
(SEQ ID NO: 96)
DQIVPEY;
E3 ligase: DCAF2
(SEQ ID NO: 97)
TSMTDFYHSKRRL;
E3 ligase: KEAP1
(SEQ ID NO: 98)
SPETGE;
E3 ligase: KLHL3
(SEQ ID NO: 99)
EPEEPEADQH;
E3 ligase: VHL
(SEQ ID NO: 100)
LAPYIPMDDDFQL;
E3 ligase: FBXW7
(SEQ ID NO: 101)
LTPPQS;
E3 ligase: SKP2/CKS1
(SEQ ID NO: 102)
SVEQTPRK;
E3 ligase: beta-TrCP1
(SEQ ID NO: 103)
DSGNYS;
E3 ligase: Siah
(SEQ ID NO: 104)
KPAAVVAPI;
or
E3 ligase: SPOP
(SEQ ID NO: 105)
ADSST

Variants of the above peptides (i.e., SEQ ID NOs.: 31-45 and 93-105) include peptides with one or more (e.g., 1, 2, 3, 4, 5) amino acid substitutions; one or more deletions (e.g., 1, 2, 3); one or more insertions (e.g., 1, 2, 3); or a combination of any two or more thereof. The variants that interact with the relevant E3 ligase are selected. In some instances, the selected peptide degron binds its relevant E3 ligase with a binding affinity of 1 nM to 300 nM. In some instances, the selected peptide degron binds the relevant E3 ligase with a binding affinity of 10 nM to 300 nM. In some instances, the selected peptide degron binds the relevant E3 ligase with a binding affinity of 50 nM to 300 nM. In some instances, the selected peptide degron binds the relevant E3 ligase with a binding affinity of 100 nM to 300 nM. In some instances, the selected peptide degron binds the relevant E3 ligase with a binding affinity of 200 nM to 300 nM. In some instances, the selected peptide degron binds the relevant E3 ligase with a binding affinity of 200 nM to 250 nM. In some instances, the selected peptide degron bins the relevant E3 ligase with a binding affinity of 1 nM to 1000 nM. In some instances, the selected peptide degron bins the relevant E3 ligase with a binding affinity of 200 nM to 1000 nM.

Non-limiting exemplary variants are provided below:

for FBXW7 E3 ligase:
(SEQ ID NO: 106)
LTPPAS,
(SEQ ID NO: 107)
LTPPSS,
(SEQ ID NO: 108)
LSPPPS,
(SEQ ID NO: 109)
LSPPAS,
(SEQ ID NO: 110)
LSPPLS;
for (β-TrCP1 E3 ligase:
(SEQ ID NO: 111)
DSGIIS,
(SEQ ID NO: 112)
DSGNYT,
(SEQ ID NO: 113)
DSGIDT,
(SEQ ID NO: 114)
DSGIET,
(SEQ ID NO: 115)
DSGVDTS;
and
for DCAF2 E3 ligase:
(SEQ ID NO: 116)
TSMTDFYHSKRRI,
(SEQ ID NO: 117)
TSMTDFYHSKRKL,
(SEQ ID NO: 118)
TSMTDFYHSKRRS

In certain instances, the peptide degron is 4 to 20 amino acids in length (e.g., 4, 5, 6, 7, 8, 9, 10, 11, 12, 13, 14, 15, 16, 17, 18, 19, or 20).

In certain instances, the peptide degron has the amino acid sequence of a peptide set forth in any one of SEQ ID NOs.: 26 to 30. In some instances, the peptide degron has an amino acid sequence that is a variant of a peptide set forth in any one of SEQ ID NOs.: 26 to 30. In certain instances, the peptide degron has the amino acid sequence of a peptide set forth in any one of SEQ ID NOs.: 31 to 45. In some instances, the peptide degron has an amino acid sequence that is a variant of a peptide set forth in any one of SEQ ID NOs.: 31 to 45. In certain instances, the peptide degron has the amino acid sequence of a peptide set forth in any one of SEQ ID NOs.: 65 to 118. In some instances, the peptide degron has an amino acid sequence that is a variant of a peptide set forth in any one of SEQ ID NOs.: 65 to 118. Variants include peptide degrons with one or more (e.g., 1, 2, 3, 4, 5) amino acid substitutions; one or more deletions (e.g., 1, 2, 3); one or more insertions (e.g., 1, 2, 3); or a combination of any two or more thereof.

The above-described peptide degrons bind to their relevant substrate adaptor for an ubiquitin E3 ligase (e.g., Cop1, FBXW7, FBXW8). In some instances, the peptide degron binds to the substrate adaptor for an ubiquitin E3 ligase (e.g., Cop1, FBXW7, FBXW8) with a binding affinity of 1 nM to 300 nM. In some instances, the peptide degron binds to the substrate adaptor for an ubiquitin E3 ligase (e.g., Cop1, FBXW7, FBXW8) with a binding affinity of 10 nM to 300 nM. In some instances, the peptide degron binds to the substrate adaptor for an ubiquitin E3 ligase (e.g., Cop1, FBXW7, FBXW8) with a binding affinity of 50 nM to 300 nM. In some instances, the peptide degron binds to the substrate adaptor for an ubiquitin E3 ligase (e.g., Cop1, FBXW7, FBXW8) with a binding affinity of 100 nM to 300 nM. In some instances, the peptide degron binds to the substrate adaptor for an ubiquitin E3 ligase (e.g., Cop1, FBXW7, FBXW8) with a binding affinity of 200 nM to 300 nM. In some instances, the peptide degron binds to the substrate adaptor for an ubiquitin E3 ligase (e.g., Cop1, FBXW7, FBXW8) with a binding affinity of 200 nM to 250 nM. In some instances, the peptide degron binds to the substrate adaptor for an ubiquitin E3 ligase (e.g., Cop1, FBXW7, FBXW8) with a binding affinity of 1 nM to 1000 nM. In some instances, the peptide degron binds to the substrate adaptor for an ubiquitin E3 ligase (e.g., Cop1, FBXW7, FBXW8) with a binding affinity of 200 nM to 1000 nM.

The disclosure also features methods of selecting a protein degron. Such selected protein degrons can be employed in the chimeric constructs of this disclosure. The method involves contacting the substrate adaptor for an ubiquitin E3 ligase with a variant of the naturally occurring peptide degron and selecting a degron that binds to the substrate adaptor with a desired affinity. For example, the method comprises contacting a WD40-repeat protein that is the substrate adaptor for an E3 ubiquitin ligase with a variant of the amino acid sequence of a natural binding consensus sequence that binds the WD40-repeat protein (e.g., Cop, FBXW7, FBXW8) and selecting a peptide that binds to the WD40-repeat protein. In some instances, the selected peptide degron binds the WD40-repeat protein with a binding affinity of 1 nM to 1000 nM. In some instances, the selected peptide degron binds the WD40-repeat protein with a binding affinity of 10 nM to 300 nM. In some instances, the selected peptide degron binds the WD40-repeat protein with a binding affinity of 50 nM to 300 nM. In some instances, the selected peptide degron binds the WD40-repeat protein with a binding affinity of 100 nM to 300 nM. In some instances, the selected peptide degron binds the WD40-repeat protein with a binding affinity of 200 nM to 300 nM. In some instances, the selected peptide degron binds the WD40-repeat protein with a binding affinity of 200 nM to 250 nM. In some instances, the selected peptide degron binds the WD40-repeat protein with a binding affinity of 1 nM to 1000 nM. In some instances, the selected peptide degron binds the WD40-repeat protein with a binding affinity of 200 nM to 1000 nM. In some instances, the selected peptide degron binds the WD40-repeat protein with a binding affinity of less than 1 nM (e.g., about 0.01 nM, about 0.05 nM, about 0.1 nM, about 0.5 nM). In some instances, the selected peptide degron binds the WD40-repeat protein with a binding affinity of greater than 300 nM (e.g., about 350 nM, about 400 nM, about 500 nM, about 1000 nM).

In certain instances, a protein that is desired to be targeted for degradation is directly modified. The protein is examined for a region that includes a structurally disordered region. A structurally disordered region is one that as calculated by IUPred: iupred.enzim.hu has a disorder score of less than or equal to 0.4. The protein is modified in the structurally disordered region to include a peptide degron sequence described above or a variant thereof. In some instances, the structurally disordered region is at the N- or C-terminus of the protein. In some instances the structurally disordered region is between the N- and C-terminus of the protein. The peptide degron sequence can be inserted into the structurally disordered region. In other cases, the peptide degron sequence is substituted to replace an amino acid sequence in the structurally disordered region of the protein. Such substitutions can be made, e.g., by CRISPR/Cas9 modification.

### Small Molecule Degrons

This disclosure features small molecule degrons that can be utilized in the chimeras described herein. In one embodiment, the degron is based on a thalidomide having the structure provided below:

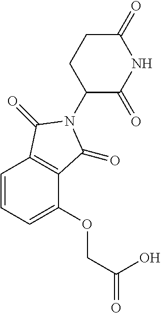 [SMILES: O=C(O)COc1cccc2c1C(=O)N(C1CCC(=O)NC1=O)C2=O]

In certain instances, when the small molecule degron is conjugated at the N-terminus of the stabilized peptide, it has the structure shown below:

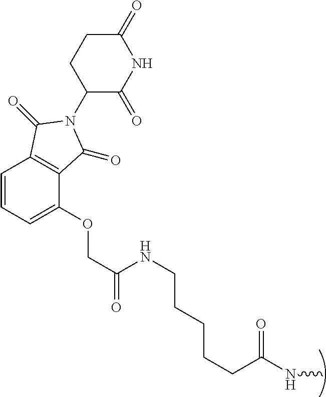

In certain instances, when the small molecule degron is conjugated at the C-terminus of the stabilized peptide, it has the structure shown below:

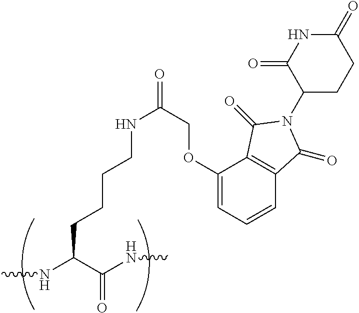

In certain instances, the small molecule degron employed herein is based on a ligand that binds the Von Hippel-Lindau (“VHL”) protein and has the structure provided below (compatible with coupling to acid residues):

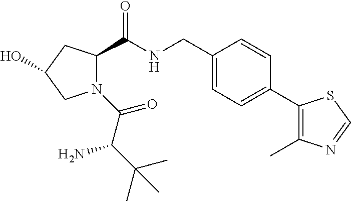 [SMILES: Cc1ncsc1-c1ccc(CNC(=O)[C@@H]2C[C@@H](O)CN2C(=O)[C@@H](N)C(C)(C)C)cc1]

In certain instances, when the small molecule VHL degron is conjugated to an amine the carboxylate analog pictured below is employed:

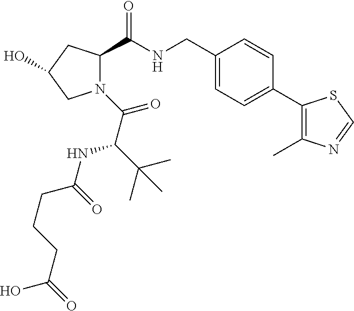 [SMILES: Cc1ncsc1-c1ccc(CNC(=O)[C@@H]2C[C@@H](O)CN2C(=O)[C@@H](NC(=O)CCCC(=O)O)C(C)(C)C)cc1]

Any small molecule degron known in the art can be employed in a chimera described herein. In certain instances, the small molecule degron employed herein is any degron described in U.S. Pat. Nos. 9,694,084; 9,750,816; 9,770,512; 9,821,068; 9,783,575; 9,765,019; 9,632,089; and 9,500,653, the contents of all of which are incorporated by reference in their entirety herein

### Chimeras of Stabilized Peptide and Degrons

This disclosure provides chimeras of stabilized peptides (e.g., stapled, stitched) with degrons (e.g., small molecule degrons, primary sequence degrons, and stabilized (e.g., stapled) peptide degrons). Such chimeras can effectively target a broad range of proteins, previously inaccessible to small molecules, with a small molecule (e.g., a cereblon-binding molecule), or other small molecule or peptide degron moiety capable of targeted degradation of bound proteins. Likewise, stabilized peptide degrons that can bind and recruit degrader proteins can be combined with small molecules that bind a broad range of proteins to degrade disease-related proteins. These new classes of stapled peptide degron chimeras expand the potency and breadth of biological activity of stapled peptides. Also encompassed herein are chimeras comprising more than one (e.g., 2, 3, 4, or more) stabilized peptide and one degron (e.g., one small molecule degron, one primary sequence degron, or one stabilized (e.g., stapled) peptide degron). Also encompassed herein are chimeras comprising more than one (e.g., 2, 3, 4, or more) degron (e.g., more than one small molecule degron, more than one primary sequence degron, or more than one stabilized (e.g., stapled) peptide degron) and one stabilized peptide. Also encompassed herein are chimeras comprising more than one (e.g., 2, 3, 4, or more) stabilized peptide and more than one (e.g., 2, 3, 4, or more) degron (e.g., more than one small molecule degron, more than one primary sequence degron, or more than one stabilized (e.g., stapled) peptide degron).

In certain embodiments, the stapled peptide of the chimera is not a Bcl-2 homology 3 (BH3) domain polypeptide (e.g., not a BH3 domain from MCL-1, not an MCL-1 Stabilized Alpha Helix of BCL2 domain (SAHB), or not MCL-1 SAHBD).

Stabilized Peptide-Peptide Degron Chimeras

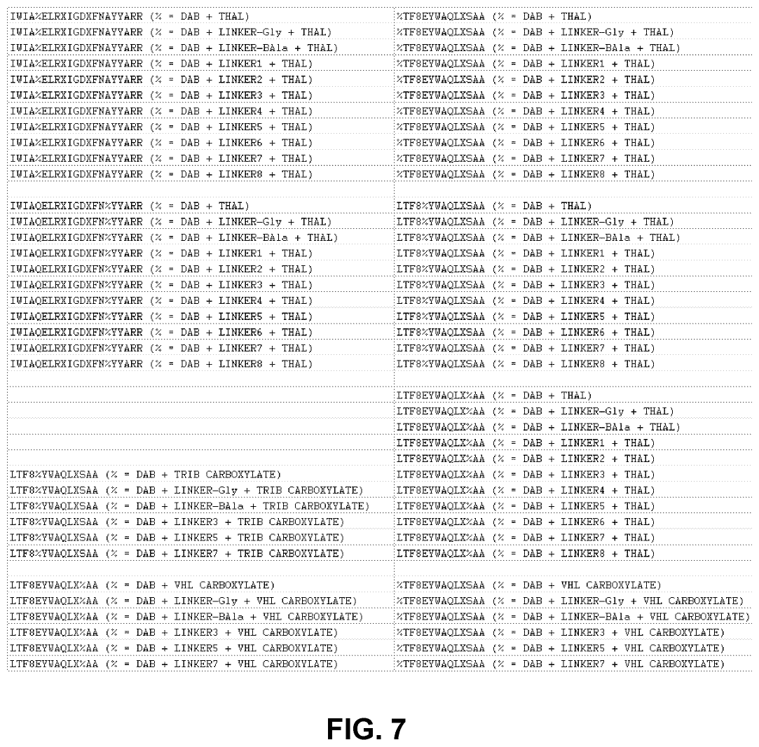

Provided herein are stabilized peptide-peptide degron chimeras. These chimeras are composed of a stabilized peptide and peptide degron, wherein the stabilized peptide binds to a first protein which is the protein targeted for degradation, and the peptide degron binds, directly or indirectly, a second protein that is the substrate adaptor for an ubiquitin E3 ligase. Thus, in certain embodiments, a stabilized peptide (e.g., stapled, stitched) described above is linked to a peptide degron described above. Exemplary chimeras are shown in FIGS. 7, 15, 17, and 18.

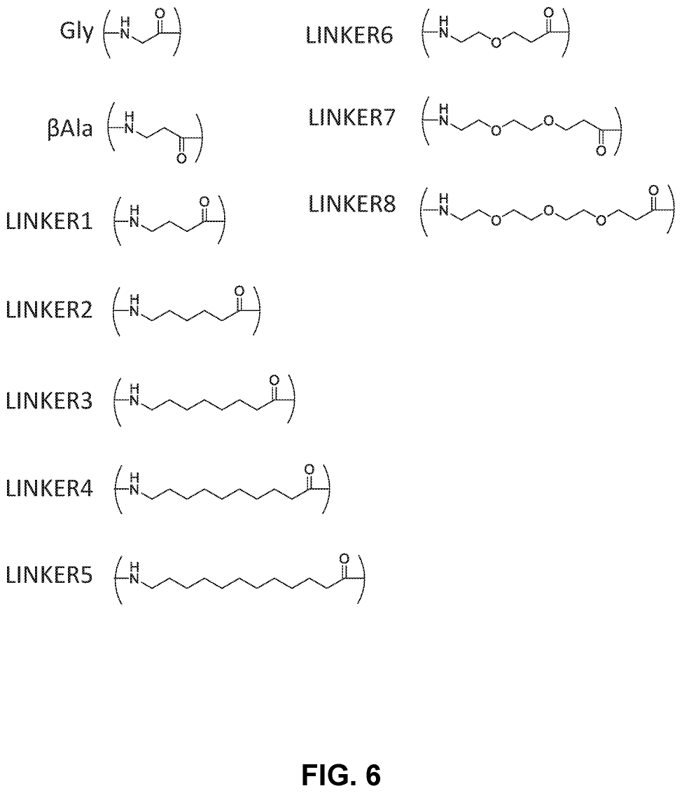

The stabilized peptide can be linked to the degron by any linker of interest (e.g., peptide linker, synthetic compound linker). Nonlimiting examples of linkers that can be used to link a peptide degron to a stabilized peptide to form a chimera described herein are described below and a subset are depicted in FIG. 6.

In certain embodiments, the stabilized peptide has an amino acid sequence set forth in any one of SEQ ID NOs.: 1-24 and 134, or a variant thereof. In some embodiments, the peptide degron has an amino acid sequence set forth in any one of SEQ ID NOs.:25-46, 65-118, or a variant thereof. In certain instances, the peptide degron is attached to the N-terminus of the stabilized peptide. In other instances, the peptide degron is attached to the C-terminus of the stabilized peptide. In some cases, a degron or degrons are attached to both the N- and C-terminus of the stabilized peptide. In some cases, a degron is attached to an internal amino acid position of the stabilized peptide (i.e., any amino acid position in the stabilized peptide except for the N- or C-terminus, e.g., position 2, 3, 4, 5, 6, 7, 8, 9, etc.). In some cases, more than one (e.g., 2 or 3) degron is attached to the stabilized peptide. In some cases in which more than one (e.g., 2 or 3) degron is attached to the stabilized peptide, one degron may be attached to a terminus of the stabilized peptide and one degron may be attached to an internal position of the stabilized peptide. In some cases in which more than one (e.g., 2 or 3) degron is attached to the stabilized peptide, one degron may be attached to each terminus of the stabilized peptide. In some cases in which more than one (e.g., 2 or 3) degron is attached to the stabilized peptide, each of the more than one degrons are attached to an internal position of the stabilized peptide. FIG. 7 depicts exemplary chimeras in which the degron is attached to the stabilized peptide at an internal amino acid position.

In certain instances, the stabilized peptide-peptide degron chimera has an amino acid sequence of one of SEQ ID NOs.: 119-126.

In certain embodiments, the stabilized peptide-peptide degron chimera comprises one or more (e.g., 2, 3, 4, or more) stabilized peptide and one peptide degron. In certain embodiments, the stabilized peptide-peptide degron chimera comprises one or more (e.g., 2, 3, 4, or more) peptide degron and one stabilized peptide. In certain embodiments, the stabilized peptide-peptide degron chimera comprises one or more (e.g., 2, 3, 4, or more) stabilized peptide and one or more (e.g., 2, 3, 4, or more) peptide degron.

Each of the chimeric constructs listed in FIGS. 7, 15, 17, and 18 are encompassed by the present disclosure. Variants of each of the chimeric constructs listed in FIGS. 7, 15, 17, and 18 are encompassed by the present disclosure.

In certain embodiments, the chimera is a chimera described in the Examples section below. See, e.g., Example 6 below for a non-limiting example of a stabilized peptide-peptide degron chimera.

Stabilized Peptide-Small Molecule Degron Chimeras

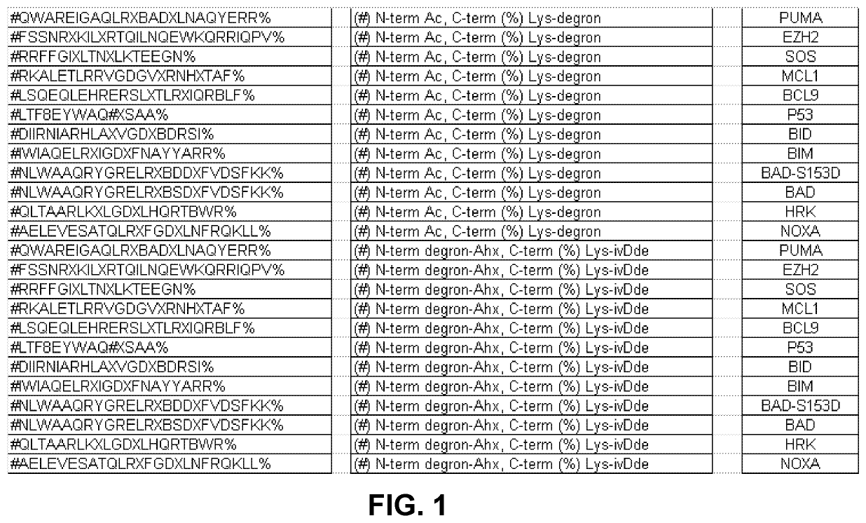

Provided herein are stabilized peptide-small molecule degron chimeras. These chimeras are composed of a stabilized peptide and a small molecule degron, wherein the stabilized peptide binds to a first protein which is the target of degradation, and the small molecule degron binds to a second protein which is a degrader protein. Thus, in certain embodiments, a stabilized peptide (e.g., stapled, stitched) described above is linked to a small molecule degron described above. The stabilized peptide can be linked to the degron by any linker of interest (e.g., synthetic compound linker). Exemplary chimeras are shown in FIGS. 1, 7, 8, 9, 11, 12, 19, and 20.

In certain embodiments, the first protein is the target of degradation by the second protein or a ligand or receptor of the second protein. In some embodiments, the first protein is an intracellular protein. In some embodiments, the first protein is an extracellular protein. In some embodiments, the first protein is a cell surface protein (e.g., receptor). In some embodiments, the first protein is a disease-causing or disease-related protein. In some embodiments, the first protein is a killer protein (e.g., BAX, BAK) or a protein that is damaging to cells or that causes neurodegeneration (e.g., IgG, beta-amyloid, tau, α-synuclein, TDP-43, HbS (hemoglobin-sickle cell), superoxide dismutase, Notch3, FUS, GFAP). In some embodiments, the first protein is a protein selected from the group consisting of BCL2, BCLXL, MCL-1, BFL-1, BCL-w, BCL-B, EZH2, HDM2/HDMX, KRAS/NRAS/HRAS, MYC, O-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In some embodiments, the first protein is a bacterial protein. In some embodiments, the first protein is a viral protein. In certain instances, the first protein is a protein aggregate (e.g., beta-amyloid) that causes neurodegeneration.

In certain embodiments, the stabilized peptide has an amino acid sequence set forth in any one of SEQ ID NOs.: 1-24, and 134, or a variant thereof.

In certain embodiments, the stabilized peptide is attached to the small molecule degron via a linker. Nonlimiting examples of linkers that can be used to attach the stabilized peptide and small molecule to each other to form a chimera described herein are described below and a subset are depicted in FIG. 6.

In certain embodiments, the stabilized peptide is indirectly attached to the small molecule peptide.

In certain instances, the small molecule degron is attached to the N-terminus of the stabilized peptide. In other instances, the small molecule degron is attached to the C-terminus of the stabilized peptide. In some cases, a degron or degrons are attached to both the N- and C-terminus of the stabilized peptide. In some cases, the degron is attached to an internal amino acid position of the stabilized peptide (i.e., any amino acid position in the stabilized peptide except for the N- or C-terminus, e.g., position 2, 3, 4, 5, 6, 7, 8, 9, etc.). In some cases, more than one (e.g., 2 or 3) degron is attached to the stabilized peptide. In some cases in which more than one (e.g., 2 or 3) degron is attached to the stabilized peptide, one degron may be attached to a terminus of the stabilized peptide and one degron may be attached to an internal position of the stabilized peptide. In some cases in which more than one (e.g., 2 or 3) degron is attached to the stabilized peptide, one degron may be attached to each terminus of the stabilized peptide. In some cases in which more than one (e.g., 2 or 3) degron is attached to the stabilized peptide, each of the more than one degrons are attached to an internal position of the stabilized peptide.

In certain embodiments, the second protein is a degrader protein, such as, e.g., an E3 ubiquitin ligase or a substrate adaptor for an E3 ubiquitin ligase. In certain embodiments, the second protein is an E3 ubiquitin ligase. Nonlimiting examples of E3 ubiquitin ligases include VHL, COP1, and MDM2. In certain embodiments, the second protein is selected from the group consisting of MDM2, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1. In certain embodiments, the second protein is a protein that binds to a protein selected from the group consisting of MDM2, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1. In some embodiments, the second protein binds to MDM2 or a protein that is complexed to MDM2, such as MDMX.

In certain embodiments, the second protein is a degrader protein, such as, e.g., an E3 ubiquitin ligase or a substrate adaptor for an E3 ubiquitin ligase. In certain embodiments, the second protein is an E3 ubiquitin ligase. In some embodiments, the second protein binds to E3 ligase (e.g., MDM2) or a protein that is complexed to an E3 ligase, such as MDMX binding to MDM2. In certain embodiments, the E3 ubiquitin ligase is a RING E3 ubiquitin ligase (e.g., Mdm2-MdmX, TRIM5α, c-CBL, cIAP, RNF4, BIRC7, IDOL, BRCA1-BARD1, RING1B-Bmi1, E4B, CHIP, Prp19). In certain embodiments, the E3 ubiquitin ligase is a HECT E3 ubiquitin ligase (e.g., Smurf1, Smurf2, Itch, E6AP). In certain embodiments, the E3 ubiquitin ligase is a RBR E3 ubiquitin ligase (e.g., Parkin, Parc, RNF144 (A/B), HOIP, HHARI). See, e.g., Morreale and Walden, Cell 165, 2016 DOI http:/dx.doi.org/10.1016/j.cell.2016.03.003 for non-limiting examples of E3 ubiquitin ligases.

In certain embodiments, the small molecule degron is based on thalidomide (see, e.g., the structure above). In certain embodiments, the small molecule degron is based on a ligand that binds the Von Hippel-Lindau protein (see, e.g., the structure above). In certain embodiments, the small molecule degron is any degron known in the art. In certain embodiments, the small molecule degron employed herein is any degron described in U.S. Pat. Nos. 9,694,084; 9,750,816; 9,770,512; 9,821,068; 9,783,575; 9,765,019; 9,632,089; and 9,500,653, the contents of all of which are incorporated by reference in their entirety herein.

Non-limiting examples of stapled peptide-small molecule degron chimeras are provided below:

PUMA
(SEQ ID NO: 1)
\hat{ }QWAREIGAQLRX₁BADX₂LNAQYERR
EZH2
(SEQ ID NO: 2)
\hat{ }FSSNRX₁KILX₂RTQILNQEWKQRRIQPV
SOS
(SEQ ID NO: 3)
\hat{ }RRFFGIX₁LTNX₂LKTEEGN
MCL-1
(SEQ ID NO: 4)
\hat{ }RKALETLRRVGDGVX₁RNHX₂TAF
BCL9
(SEQ ID NO: 5)
\hat{ }LSQEQLEHRERSLVTLRX₂IQRBLF
p53
(SEQ ID NO: 6)
\hat{ }LTF8EYWAQ#XSAA
BID
(SEQ ID NO: 7)
\hat{ }DIIRNIARHLAX₁VGDX2BDRSI
BIM
(SEQ ID NO: 8)
\hat{ }IWIAQELRVIGDX₂FNAYYARR
BAD S153D
(SEQ ID NO: 9)
\hat{ }NLWAAQRYGRELRX₁BDDX₂FVDSFKK
BAD
(SEQ ID NO: 10)
\hat{ }NLWAAQRYGRELRX₁BSDX₂FVDSFKK
HRK
(SEQ ID NO: 11)
\hat{ }QLTAARLKX₁LGDX₂LHQRTBWR
NOXA
(SEQ ID NO: 12)
\hat{ }AELEVESATQLRX₁FGDX₂LNFRQKLL
PUMA
(SEQ ID NO: 13)
QWAREIGAQLRX₁BADX₂LNAQYERR&
EZH2
(SEQ ID NO: 14)
FSSNRX₁KILX₂RTQILNQEWKQRRIQPV&
SOS
(SEQ ID NO: 15)
RRFFGIX₁LTNX₂LKTEEGN&
MCL-1
(SEQ ID NO: 16)
RKALETLRRVGDGVX₁RNHX₂TAF&
BCL9
(SEQ ID NO: 17)
LSQEQLEHRERSLX₁TLRX₂IQRBLF&
p53
(SEQ ID NO: 18)
LTF8EYWAQ#XSAA&
BID
(SEQ ID NO: 19)
DIIRNIARHLAVVGDX₂BDRSI&
BIM
(SEQ ID NO: 20)
IWIAQELRVIGDX₂FNAYYARR&
BAD-S153D
(SEQ ID NO: 21)
NLWAAQRYGRELRX₁BDDX₂FVDSFKK&
BAD
(SEQ ID NO: 22)
NLWAAQRYGRELRX₁BSDX₂FVDSFKK&
HRK
(SEQ ID NO: 23)
QLTAARLKX₁LGDX₂LHQRTBWR&
NOXA
(SEQ ID NO: 24)
AELEVESATQLRX₁FGDX₂LNFRQKLL&
BIM
(SEQ ID NO: 136)
IWIA%ELRX₁IGDX₂FNAYYARR
BIM
(SEQ ID NO: 137)
IWIAQELRX₁IGDX₂FN%YYARR
(SEQ ID NO: 138)
LTF8$YWAQLXSAA
(SEQ ID NO: 139)
LTF8EYWAQLX$AA
(SEQ ID NO: 140)
%TF8EYWAQLXSAA
(SEQ ID NO: 141)
LTF8%YWAQLXSAA
(SEQ ID NO: 142)
LTF8EYWAQLX%AA
(SEQ ID NO: 143)
@TF8EYWAQLXSAA,

wherein, 8=R-octenyl alanine; B=norleucine; #=cyclobutylalanine; X=S-pentenyl alanine, and, in some instances, X₁ and X₂ are the same (e.g., S-penetenyl alanine), \hat{ }=thalidomide-aminohexanoic, &=Lys-epsilon-amino-thalidomide, %=A₁, A₂, A₃, A₄, A₅, A₆, A₇, A₈, A₉, A₁₀, or A₁₁, wherein A₁=DAB-thalidomide, A₂=DAB-Gly-Thal, A₃=DAB-βAla-Thal, A₄=DAB-Linker1-Thal, A₅=DAB-Linker2-Thal, A₆=DAB-Linker3-Thal, A₇=DAB-Linker4-Thal, A₈=DAB-Linker5-Thal, A₉=DAB-Linker6-Thal, A₁₀=DAB-Linker7-Thal, and A₁₁=DAB-Linker8-Thal; $=B₁, B₂, B₃, B₄, B₅, or B₆, wherein B₁=DAB-TRIB carboxylate, B₂=DAB-Gly-TRIB carboxylate, B₃=DAB-βAla-TRIB carboxylate, B₄=DAB-Linker3-TRIB carboxylate, B₅=DAB-Linker5-TRIB carboxylate, and B₆=DAB-Linker7-TRIB carboxylate; @=C₁, C₂, C₃, C₄, C₅, or C₆, wherein C₁=DAB-VHL carboxylate, C₂=DAB-Gly-VHL carboxylate, C₃=DAB-βAla-VHL carboxylate, C₄=DAB-Linker3-VHL carboxylate, C₅=DAB-Linker5-VHL carboxylate, and C₆=DAB-Linker7-VHL carboxylate,
wherein, Linker1-Linker8 are depicted in FIG. 6. In some embodiments, the thalidomide-aminohexanoic (\hat{ }) or the Lys-epsilon-amino-thalidomide (&) are linked to the stapled peptide via a linker.

In certain embodiments, the stabilized peptide-small molecule degron chimera comprises one or more (e.g., 2, 3, 4, or more) stabilized peptide and one small molecule degron. In certain embodiments, the stabilized peptide-small molecule degron chimera comprises one or more (e.g., 2, 3, 4, or more) small molecule degron and one stabilized peptide. In certain embodiments, the stabilized peptide-small molecule degron chimera comprises one or more (e.g., 2, 3, 4, or more) stabilized peptide and one or more (e.g., 2, 3, 4, or more) small molecule degron.

Each of the above-listed chimeric constructs and variants thereof are encompassed by the present disclosure. Each of the chimeric constructs listed in FIGS. 1, 7, 8, 9, 11, 12, 19, and 20 are encompassed by the present disclosure. Variants of each of the chimeric constructs listed in FIGS. 1, 7, 8, 9, 11, 12, 19, and 20 are encompassed by the present disclosure.

In certain embodiments, the chimera is a chimera described in the Examples section below. See, e.g., Examples 2, 3, 4, and 7 below for non-limiting examples of a stabilized peptide-small molecule degron chimera.

Stabilized Peptide-Stabilized Peptide Degron Chimeras

Provided herein are stabilized peptide-stabilized peptide degron chimeras. These chimeras are composed of two stabilized peptides-a first stabilized peptide and a second stabilized peptide—wherein the first stabilized peptide binds to a first protein that is the protein target to be degraded, and the second stabilized peptide binds to a second protein that is a degrader protein. Thus, in certain embodiments, a first stabilized peptide (e.g., stapled, stitched) described above is linked to a second stabilized peptide (e.g., stapled, stiched) described above. The first and second stabilized peptides can be linked directly or indirectly.

In certain embodiments, the first protein is the protein target to be degraded by the second protein or a ligand or receptor of the second protein. In some embodiments, the first protein is an intracellular protein. In some embodiments, the first protein is an extracellular protein. In some embodiments, the first protein is a cell surface protein (e.g., receptor). In some embodiments, the first protein is a disease-causing or disease-related protein. In some embodiments, the first protein is a killer protein (e.g., BAX, BAK) or a protein that is damaging to cells or that causes neurodegeneration (e.g., IgG, beta-amyloid, tau, α-synuclein, TDP-43, HbS (hemoglobin-sickle cell), superoxide dismutase, Notch3, FUS, GFAP). In some embodiments, the first protein is a protein selected from the group consisting of BCL2, BCLXL, MCL-1, BFL-1, BCL-w, BCL-B, EZH2, HDM2/HDMX, KRAS/NRAS/HRAS, MYC, O-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In some embodiments, the first protein is a bacterial protein. In some embodiments, the first protein is a viral protein. In certain instances, the first protein is a protein aggregate (e.g., beta-amyloid) that causes neurodegeneration.

In certain embodiments, the first stabilized peptide has an amino acid sequence set forth in any one of SEQ ID NOs.: 1-24, and 134, or a variant thereof.

In certain embodiments, the first stabilized peptide is attached to the second stabilized peptide via a linker. Nonlimiting examples of linkers that can be used to attach the first and second stabilized peptides to each other to form a chimera described herein are described below and a subset are depicted in FIG. 6.

In certain embodiments, the first stabilized peptide is indirectly attached to the second stabilized peptide.

In certain embodiments, the second protein is a degrader protein, such as, e.g., an E3 ubiquitin ligase or a substrate adaptor for an E3 ubiquitin ligase. In certain embodiments, the second protein is an E3 ubiquitin ligase. Nonlimiting examples of E3 ubiquitin ligases include VHL, COP1, and MDM2. In certain embodiments, the second protein is selected from the group consisting of MDM2, MDMX, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1. In certain embodiments, the second protein is a protein that binds to a protein selected from the group consisting of MDM2, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1. In some embodiments, the second protein binds to MDM2 or a protein that is complexed to MDM2, such as MDMX.

In certain embodiments, the second protein is a degrader protein, such as, e.g., an E3 ubiquitin ligase or a substrate adaptor for an E3 ubiquitin ligase. In certain embodiments, the second protein is an E3 ubiquitin ligase. In some embodiments, the second protein binds to E3 ligase (e.g., MDM2) or a protein that is complexed to an E3 ligase, such as MDMX binding to MDM2. In certain embodiments, the E3 ubiquitin ligase is a RING E3 ubiquitin ligase (e.g., Mdm2-MdmX, TRIM5α, c-CBL, cIAP, RNF4, BIRC7, IDOL, BRCA1-BARD1, RING1B-Bmi1, E4B, CHIP, Prp19). In certain embodiments, the E3 ubiquitin ligase is a HECT E3 ubiquitin ligase (e.g., Smurf1, Smurf2, Itch, E6AP). In certain embodiments, the E3 ubiquitin ligase is a RBR E3 ubiquitin ligase (e.g., Parkin, Parc, RNF144 (A/B), HOIP, HHARI). See, e.g., Morreale and Walden, Cell 165, 2016 DOI http:/dx.doi.org/10.1016/j.cell.2016.03.003 for non-limiting examples of E3 ubiquitin ligases.

In certain embodiments, the second stabilized peptide has an amino acid sequence set forth in SEQ ID NO: 134, or a variant thereof. In certain embodiments, the second stabilized peptide has an amino acid sequence set forth in SEQ ID NO: 6, or a variant thereof. In certain embodiments, the second stabilized peptide has an amino acid sequence set forth in SEQ ID NO: 18, or a variant thereof.

In certain embodiments, the second stabilized peptide is a stabilized peptide described in U.S. Pat. Nos. 8,889,632, 9,458,202, 9,505,804, 9,527,896, 9,957,299, 10,030,049, and 10,059,741, International Patent Application Publication Nos. WO 1998/001467 and WO 2017/165617, and US Patent Application Publication No. 2014/0018302A₁, each of which is incorporated by reference herein in its entirety.

In certain instances, the first stabilized peptide is attached to the N-terminus of the second stabilized peptide. In other instances, the first stabilized peptide is attached to the C-terminus of the second stabilized peptide. In some cases, the first stabilized peptide is attached to an internal amino acid position of the second stabilized peptide (i.e., any amino acid position in the stabilized peptide except for the N- or C-terminus, e.g., position 2, 3, 4, 5, 6, 7, 8, 9, etc.). In certain instances, the second stabilized peptide is attached to the N-terminus of the first stabilized peptide. In other instances, the second stabilized peptide is attached to the C-terminus of the first stabilized peptide. In some cases, the second stabilized peptide is attached to an internal amino acid position of the first stabilized peptide (i.e., any amino acid position in the stabilized peptide except for the N- or C-terminus, e.g., position 2, 3, 4, 5, 6, 7, 8, 9, etc.).

In certain embodiments, the stabilized peptide-stabilized peptide degron chimera comprises one or more (e.g., 2, 3, 4, or more) stabilized peptide that binds to one or more proteins to be degraded and one stabilized peptide degron that binds to a degrader protein. In certain embodiments, the stabilized peptide-stabilized peptide degron chimera comprises one or more (e.g., 2, 3, 4, or more) stabilized peptide degrons that bind to one or more degrader proteins and one stabilized peptide that binds to a protein to be degraded. In certain embodiments, the stabilized peptide-stabilized peptide degron chimera comprises one or more (e.g., 2, 3, 4, or more) stabilized peptide that binds to one or more proteins to be degraded and one or more (e.g., 2, 3, 4, or more) stabilized peptide degrons that bind to one or more degrader proteins.

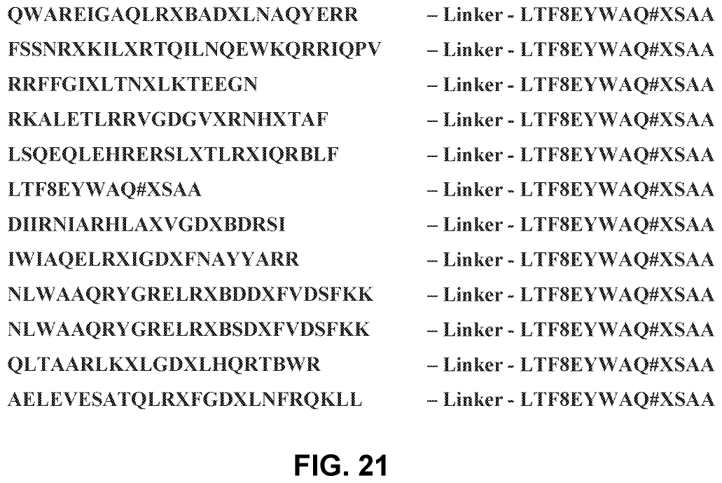

Each of the chimeric constructs listed in FIG. 21 are encompassed by the present disclosure. Variants of each of the chimeric constructs listed in FIG. 21 are encompassed by the present disclosure.

In certain embodiments, the chimera is a chimera described in the Examples section below. See, e.g., Example 8 below for a non-limiting example of a stabilized peptide-stabilized peptide degron chimera.

Small Molecule-Stabilized Peptide Degron Chimeras

Provided herein are small molecule-stabilized peptide degron chimeras. These chimeras are composed of a small molecule and a stabilized peptide, wherein the small molecule binds to a first protein that is the protein target to be degraded, and the stabilized peptide binds to a second protein, which is a degrader protein. Thus, in certain embodiments, a small molecule is linked to a stabilized peptide (e.g., stapled, stiched) described above. The small molecule and the stabilized peptides can be linked directly or indirectly.

In certain embodiments, the first protein is the protein target to be degraded by the second protein or a ligand or receptor of the second protein. In some embodiments, the first protein is an intracellular protein. In some embodiments, the first protein is an extracellular protein. In some embodiments, the first protein is a cell surface protein (e.g., receptor). In some embodiments, the first protein is a disease-causing or disease-related protein. In some embodiments, the first protein is a killer protein (e.g., BAX, BAK) or a protein that is damaging to cells or that causes neurodegeneration (e.g., IgG, beta-amyloid, tau, α-synuclein, TDP-43, HbS (hemoglobin-sickle cell), superoxide dismutase, Notch3, FUS, GFAP). In some embodiments, the first protein is a protein selected from the group consisting of BCL2, BCLXL, MCL-1, BFL-1, BCL-w, BCL-B, EZH2, HDM2/HDMX, KRAS/NRAS/HRAS, MYC, O-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In some embodiments, the first protein is a bacterial protein. In some embodiments, the first protein is a viral protein. In certain instances, the first protein is a protein aggregate (e.g., beta-amyloid) that causes neurodegeneration.

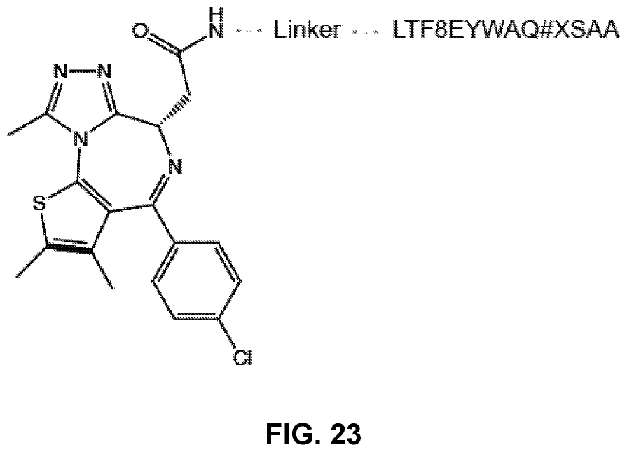

In certain embodiments, the small molecule is any drug or chemical compound that, when forming part of the chimera, is able to bind to a protein without interfering with the ability of the stapled peptide of the chimera to interact with its target. Assays and methods are known in the art for evaluating a drug's or a chemical compound's interference (in its context of the chimera) with the ability of the stapled peptide (of the chimera) to interact with its target such as, e.g., immunofluorescence and co-immunoprecipitation. In certain embodiments, the small molecule is the compound depicted in FIG. 23. In certain embodiments, the small molecule is a kinase inhibitor. In certain embodiments, the small molecule is a histone deacetylase inhibitor.

In certain embodiments, the small molecule is attached to the stabilized peptide via a linker. Nonlimiting examples of linkers that can be used to attach the small molecule and the stabilized peptide to each other to form a chimera described herein are described below and a subset are depicted in FIG. 6.

In certain embodiments, the small molecule is indirectly attached to the stabilized peptide.

In certain instances, the small molecule is attached to the N-terminus of the stabilized peptide. In other instances, the small molecule is attached to the C-terminus of the stabilized peptide. In some cases, the small molecule is attached to an internal amino acid position of the stabilized peptide (i.e., any amino acid position in the stabilized peptide except for the N- or C-terminus, e.g., position 2, 3, 4, 5, 6, 7, 8, 9, etc.).

In certain embodiments, the second protein is a degrader protein, such as, e.g., an E3 ubiquitin ligase or a substrate adaptor for an E3 ubiquitin ligase. In certain embodiments, the second protein is an E3 ubiquitin ligase. Nonlimiting examples of E3 ubiquitin ligases include VHL, COP1, and MDM2. In certain embodiments, the second protein is selected from the group consisting of MDM2, MDMX, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1. In certain embodiments, the second protein is a protein that binds to a protein selected from the group consisting of MDM2, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1. In some embodiments, the second protein binds to MDM2 or a protein that is complexed to MDM2, such as MDMX.

In certain embodiments, the second protein is a degrader protein, such as, e.g., an E3 ubiquitin ligase or a substrate adaptor for an E3 ubiquitin ligase. In certain embodiments, the second protein is an E3 ubiquitin ligase. In some embodiments, the second protein binds to E3 ligase (e.g., MDM2) or a protein that is complexed to an E3 ligase, such as MDMX binding to MDM2. In certain embodiments, the E3 ubiquitin ligase is a RING E3 ubiquitin ligase (e.g., Mdm2-MdmX, TRIM5a, c-CBL, cIAP, RNF4, BIRC7, IDOL, BRCA1-BARD1, RINGB-Bmi1, E4B, CHIP, Prp19). In certain embodiments, the E3 ubiquitin ligase is a HECT E3 ubiquitin ligase (e.g., Smurf1, Smurf2, Itch, E6AP). In certain embodiments, the E3 ubiquitin ligase is a RBR E3 ubiquitin ligase (e.g., Parkin, Parc, RNF144 (A/B), HOIP, HHARI). See, e.g., Morreale and Walden, Cell 165, 2016 DOI http:/dx.doi.org/10.1016/j.cell.2016.03.003 for non-limiting examples of E3 ubiquitin ligases.

In certain embodiments, the stabilized peptide has an amino acid sequence set forth in SEQ ID NO: 134, or a variant thereof. In certain embodiments, the second stabilized peptide has an amino acid sequence set forth in SEQ ID NO: 6, or a variant thereof. In certain embodiments, the second stabilized peptide has an amino acid sequence set forth in SEQ ID NO: 18, or a variant thereof.

In certain embodiments, the small molecule-stabilized peptide degron chimera comprises one or more (e.g., 2, 3, 4, or more) stabilized peptide degron and one small molecule. In certain embodiments, the small molecule-stabilized peptide degron chimera comprises one or more (e.g., 2, 3, 4, or more) small molecule and one stabilized peptide degron. In certain embodiments, the small molecule-stabilized peptide degron chimera comprises one or more (e.g., 2, 3, 4, or more) stabilized peptide degron and one or more (e.g., 2, 3, 4, or more) small molecule.

The chimeric construct depicted in FIG. 23 is encompassed by the present disclosure. Variants of the chimeric construct depicted in FIG. 23 is encompassed by the present disclosure.

In certain embodiments, the chimera is a chimera described in the Examples section below. See, e.g., Example 9 below for a non-limiting example of a small molecule-stabilized peptide degron chimeras.

### Linkers

There is no particular limitation on the linkers that can be used in the constructs described above. In some embodiments, the linker is an amino acid such as amino-propionic-acid, amino-butanoic-acid, amino-pentanoic-acid, or amino-hexanoic-acid. In some embodiments, the linker is an oligoethylene glycol, i.e., NH₂—(CH₂—CH₂—O)ₓ—CH₂—CH₂—COOH. In some embodiments, the linker is a peptide linker. In some embodiments, any arbitrary single-chain peptide comprising about one to 30 residues (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids) can be used as a linker. In other embodiments, the linker is 10 to 20, 10 to 30, 10 to 40, 10 to 50, 10 to 60, 10 to 70, 10 to 80, 10 to 90, 10 to 100, 10 to 144, or 10 to 150 amino acids in length. In certain instances, the linker contains only glycine and/or serine residues. Examples of such peptide linkers include: Gly, Ser; Gly Ser; Gly Gly Ser; Ser Gly Gly; Gly Gly Gly Ser (SEQ ID NO:47); Ser Gly Gly Gly (SEQ ID NO:48); Gly Gly Gly Gly Ser (SEQ ID NO:49); Ser Gly Gly Gly Gly (SEQ ID NO:50); Gly Gly Gly Gly Gly Ser (SEQ ID NO:51); Ser Gly Gly Gly Gly Gly (SEQ ID NO:52); Gly Gly Gly Gly Gly Gly Ser (SEQ ID NO:53); Ser Gly Gly Gly Gly Gly Gly (SEQ ID NO:54); (Gly Gly Gly Gly Ser)ₙ (SEQ ID NO:49)ₙ, wherein n is an integer of one or more; and (Ser Gly Gly Gly Gly)ₙ (SEQ ID NO:50)ₙ, wherein n is an integer of one or more. In some instances, the linker has the amino acid sequence of SEQ ID NO:4 with the exception that the serine residue is replaced with another amino acid. In some instances, the linker has multiple copies (e.g., 1, 2, 3, 4, 5, 6, 7, 8, 9, 10) of the amino acid sequence of SEQ ID NO:4 with the exception that the serine residue in each copy of the linker is replaced with another amino acid.

In other embodiments, the linker peptides are modified such that the amino acid sequence GSG (that occurs at the junction of traditional Gly/Ser linker peptide repeats) is not present. For example, the peptide linker comprise an amino acid sequence selected from the group consisting of: (GGGXX)ₙGGGGS (SEQ ID NO:55) and GGGGS(XGGGS)ₙ (SEQ ID NO:56), where X is any amino acid that can be inserted into the sequence and not result in a polypeptide comprising the sequence GSG, and n is 0 to 4. In one embodiment, the sequence of a linker peptide is (GGGX₁X₂)ₙGGGGS and X₁ is P and X₂ is S and n is 0 to 4 (SEQ ID NO:57). In another embodiment, the sequence of a linker peptide is (GGGX₁X₂)ₙGGGGS and X₁ is G and X₂ is Q and n is 0 to 4 (SEQ ID NO:58). In another embodiment, the sequence of a linker peptide is (GGGX₁X₂)ₙGGGGS and X₁ is G and X₂ is A and n is 0 to 4 (SEQ ID NO:59). In yet another embodiment, the sequence of a linker peptide is GGGGS(XGGGS)ₙ, and X is P and n is 0 to 4 (SEQ ID NO:60). In one embodiment, a linker peptide of the invention comprises or consists of the amino acid sequence (GGGGA)₂GGGGS (SEQ ID NO:61). In another embodiment, a linker peptide comprises or consists of the amino acid sequence (GGGGQ)₂GGGGS (SEQ ID NO:62). In yet another embodiment, a linker peptide comprises or consists of the amino acid sequence (GGGPS)₂GGGGS (SEQ ID NO:63). In a further embodiment, a linker peptide comprises or consists of the amino acid sequence GGGGS(PGGGS)₂(SEQ ID NO:64).

In certain embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

In certain embodiments, the linker is a linker depicted in FIG. 6.

### Methods of Synthesizing Stabilized Peptide Degron Chimeras

Synthesis of Stapled Peptide-Small Molecule Degron Chimeras

Hydrocarbon-stapled peptides can be synthesized, purified, and quantitated using previously reported methods (Bird et al., Methods Enzymol., 446:369-86 (2008); Bird et al., Curr. Protoc. Chem. Biol., 3(3):99-117 (2011), with the following modifications and added details: The peptide is synthesized using established methods (i.e., Fmoc protected amino acids, HATU coupling reagent) until the desired sequence is complete. The peptide is then stapled using Grubbs catalyst (generation 1) and the N-terminus deprotected using piperidine. A multi-atom linker is incorporated, such as beta alanine or amino hexanoic acid. The thalidomide-COOH is then coupled using HCTU. The imide bond is particularly sensitive to nucleophiles so piperidine or hydrazine is not used after incorporation. The peptides are then cleaved for 1 hour with TFA and purified by LCMS.

Synthesis of Stapled Peptide-Peptide Degron Chimeras

The stapled peptide portion of the chimera can be synthesized as above followed by the coupling of a fully protected peptide degron. The fully-protected degron peptide can be synthesized on a weak acid-cleavable resin, such as the Sieber amide resin, with the final synthetic step being reaction of glycolic anhydride with the peptide N-terminus. After 1% TFA cleavage, the protected peptide is precipitated in ether, dissolved in acetic acid/water, and lyophilized. The fully-protected degron peptide is then mixed with coupling reagent and base, and reacted with the resin-bound stapled peptide N-terminus for 2 hours, followed by TFA cleavage and purification to yield the stapled peptide-peptide degron.

Synthesis of Stapled Peptide-Stapled Peptide Degron Chimeras

The first stapled peptide portion can be synthesized using the established methods described above, followed by incorporation of a linker moiety, such as beta alanine or amino hexanoic acid. The second half of the stapled peptide chimera can then synthesized using the same protocol for the first stapled peptide portion, and then the entire chimera can be stapled using Grubbs catalyst (generation 1), followed by acetylation of the N-terminus. The chimera is then subjected to cleavage for 1 hour with TFA and purified by LCMS.

Synthesis of Small Molecule-Stapled Peptide Degron Chimeras

The stapled peptide portion of the chimera can be synthesized using the established methods described above, followed by peptide stapling using the Grubbs catalyst (generation 1), deprotection of the N-terminus with piperidine, and incorporation of a multi-atom linker, such as beta alanine or amino hexanoic acid. Coupling of the small molecule to the stapled peptide can be performed as described above.

Properties and functional activities of the stabilized (e.g., stapled) peptide degron chimeras of the invention can be assayed, for example, using the methods described below.

Binding of Stapled Peptide Degron Chimeras to Protein Targets

Competitive fluorescence polarization assays are performed to monitor the capacity of (1) the stapled peptide (or molecular) portion of a chimera to retain binding affinity for its protein target and (2) the degron component (whether molecular or peptidic) to retain binding affinity for its protein target. Exemplary fluorescence polarization methods for stapled peptides and molecular degrons include Pitter et al Methods Enzymol 446: 387-408 (2008) and Nowak et al. Nat Chem Biol 14:706-714 (2018). In cellulo degradation assays using GFP-labeled target protein substrate (e.g. GFP-BRD4) are likewise used to confirm the capacity of stapled peptide degron chimeras to penetrate intact cells and compete with positive control molecular degron chimeras (e.g. dBET6) to inhibit induced degradation. Exemplary methods for such competitive cellular degradation assays can be found in Nowak et al. Nat Chem Biol 14:706-714 (2018).

Monitoring Ubiquitination of Recombinant Protein Targets as Induced by Stapled Peptide Degron Chimeras.

To monitor ubiquitination of protein targets in vitro, a commercial Mdm2/HDM2 Ubiquitin Ligase Kit (K-200B) can be employed. Briefly, chimera (10 μM), recombinant full length MDM2 (GST-tagged, 1 μM), E1 enzyme (UBE1, 50 nM), E2 enzyme (UBE2D3, 1 μM), ubiquitin (100 μM), ATP (1 mM) and recombinant target protein (100 nM) are combined in a 1.5 mL microtube in reaction buffer. The mixture is incubated at 37 degrees Celsius for six hours. Subsequently, 20 uL of reaction mixture is assayed using standard western blotting techniques whereby antibodies raised against the target protein are used to visualize the upward band shifts due to ubiquitination.

Monitoring Native Protein Degradation in Cellulo as Induced by Stapled Peptide Degron Chimeras.

To assay for intracellular protein degradation, cancer cells (e.g., SJSA-1, SJSA-X, U20S) are passaged at 37 degrees Celsius in a humidity-controlled, CO₂-equilibrated incubator in DMEM (Life Technologies, Grand Island, N.Y.) culture medium (CM) containing 10% fetal bovine serum (FBS) and 1% penicillin/streptomycin (Pen Strep). One day before treatment, cells are passaged and plated in six well plates at a density of 100,000 cells/mL. After 24 hours, cells are treated with a stapled peptide degron chimera (e.g., 10 μM) for 0, 2, 4 and 6 hours after which they were harvested and lysed. Cell lysates are then assayed using standard western blotting techniques using an antibody raised against the target protein to assess protein level and an actin antibody for the loading control.

Monitoring the Impact of Target Protein Degradation Induced by Stapled Peptide Degron Chimeras on Cancer Cell Viability.

Stapled peptide degron chimeras that retain binding to both protein targets, achieve cellular uptake, and can access their dual targets in cellulo to induce targeted protein degradation are assessed for their cytotoxic effect on cancer cells using established cell viability and apoptosis assays, including Cell Titer Glo and caspase 3/7 activation assays, performed as reported (e.g. Labelle et al, J Clin Invest 122:2018-31 (2012); Wachter et al, Oncogene, 36:2184-2190 (2017); Guerra et al, Cell Reports 24:3393-3403 (2018). Specificity of action controls studies are performed using, for example, point mutant peptides that cannot engage their protein target and/or cell lines that do not express the target protein and/or degrader protein of interest.

### Methods of Treatment

The chimeras disclosed herein can facilitate degradation of the disease-related protein that the stabilized peptide or small molecule binds. In certain instances, the protein that is degraded is a killer proteins like BAX or BAK (this is useful for e.g., as a cytoprotectant during stress as in such as in stroke, neurodegenerative disease, and hypoxia in heart attack). In certain instances, the protein that is degraded is a protein that is damaging to cells, like Igs in myeloma, amyloid in Alzheimer's disease, other protein deposits that cause disease. In certain instances, the protein that is degraded is a protein selected from the group consisting of BCL2, /BCLX_{L}, MCL-1, BFL-1, BCL-w, BCL-B, EZH2, HDM2/HDMX, PUMA, SOSKRAS/NRAS/HRAS, MYC, b-catenin, PI3K, PTEN, TSC, AKT, BRCA1/2, EWS-FLI, MLL fusions, a receptor Tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5. In certain instances, the protein that is degraded is a protein selected from the group consisting of Amyloid beta (Alzheimer's disease), tau protein (Alzheimer's disease), alpha-synuclein (Alzheimer's disease), TDP-43 (frontotemporal lobar degeneration), superoxide dismutase (ALS), Notch3 (CADASIL), FUS (sarcoma, ALS), amyloid A, Ig heavy and light chain, and GFAP (Alexander disease).

The disclosure features methods of using any of the stabilized peptides or chimeras described herein for the prophylaxis and/or treatment of a cancer, an autoimmune disease, or an inflammatory disease. The terms “treat” or “treating,” as used herein, refers to alleviating, inhibiting, or ameliorating the disease or condition from which the subject is suffering.

The peptides or chimeras described herein can be useful for treating a human subject with a cancer. The peptides or chimeras described herein can also be useful for treating a human subject with a melanoma, a leukemia, lymphoma, or other hematologic malignancy or solid tumor. In certain instances, the solid tumor is a melanoma, a breast cancer or a lung cancer. In some embodiments, the peptides or chimeras described herein can be useful for treating a human subject with autoimmune disease or other inflammatory condition characteristic of a disease of cellular excess. In certain instances, the autoimmune disease is autoimmune colitis, thyroiditis, arthritis, nephritis, dermatitis, vasculitis, system lupus erythematosus, diabetes, or Sjogren's disease. In some instances, the inflammatory disease, asthma, psoriasis, inflammatory colitis, thyroiditis, arthritis, nephritis, dermatitis, or vasculitis.

In instances, where an endogenous protein (e.g., an oncogenic protein like MDM2) is to be modified to include a degron, any gene editing technique can be used (see, e.g., U.S. Pat. Nos. 9,840,713; 9,840,702; 9,840,699; 9,834,791; 9,822,372; 9,816,080; 9,790,490; 9,783,490; 9,771,601; 9,758,775; 9,738,908; 9,616,090; 9,574,211, all of which are incorporated by reference in their entireties herein). The presence of the newly introduced degron should lead to the protein's degradation.

In general, methods include selecting a subject and administering to the subject an effective amount of one or more of the peptides herein, e.g., in or as a pharmaceutical composition, and optionally repeating administration as required for the prophylaxis or treatment of a cancer, e.g., melanoma or lymphoma, and can be administered orally, intravenously or topically. A subject can be selected for treatment based on, e.g., determining that the subject has a cancer that expresses the protein the stapled peptide targets (e.g., MCL-1, BFL-1).

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, effective amounts can be administered at least once.

### Pharmaceutical Compositions

One or more of any of the stabilized peptides or chimeras described herein can be formulated for use as or in pharmaceutical compositions. Such compositions can be formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.htm). For example, compositions can be formulated or adapted for administration by inhalation (e.g., oral and/or nasal inhalation (e.g., via nebulizer or spray)), injection (e.g., intravenously, intra-arterial, subdermally, intraperitoneally, intramuscularly, and/or subcutaneously); and/or for oral administration, transmucosal adminstration, and/or topical administration (including topical (e.g., nasal) sprays and/or solutions).

In some instances, pharmaceutical compositions can include an effective amount of one or more stabilized peptides. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment of infection).

Pharmaceutical compositions of this invention can include one or more peptides and any pharmaceutically acceptable carrier and/or vehicle. In some instances, pharmaceuticals can further include one or more additional therapeutic agents in amounts effective for achieving a modulation of disease or disease symptoms.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

Pharmaceutical compositions can be in the form of a solution or powder for inhalation and/or nasal administration. Such compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Pharmaceutical compositions can be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Alternatively or in addition, pharmaceutical compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In some instances, one or more peptides disclosed herein can be conjugated, for example, to a carrier protein. Such conjugated compositions can be monovalent or multivalent. For example, conjugated compositions can include one peptide disclosed herein conjugated to a carrier protein. Alternatively, conjugated compositions can include two or more peptides disclosed herein conjugated to a carrier.

As used herein, when two entities are “conjugated” to one another they are linked by a direct or indirect covalent or non-covalent interaction. In certain embodiments, the association is covalent. In other embodiments, the association is non-covalent. Non-covalent interactions include hydrogen bonding, van der Waals interactions, hydrophobic interactions, magnetic interactions, electrostatic interactions, etc. An indirect covalent interaction is when two entities are covalently connected, optionally through a linker group.

Carrier proteins can include any protein that increases or enhances immunogenicity in a subject. Exemplary carrier proteins are described in the art (see, e.g., Fattom et al., Infect. Immun., 58:2309-2312, 1990; Devi et al., Proc. Nal. Acad. Sci. USA 88:7175-7179, 1991; Li et al., Infect. Immun. 57:3823-3827, 1989; Szu et al., Infect. Immun. 59:4555-4561, 1991; Szu et al., J Exp. Med. 166:1510-1524, 1987; and Szu et al., Infect. Immun. 62:4440-4444, 1994). Polymeric carriers can be a natural or a synthetic material containing one or more primary and/or secondary amino groups, azido groups, or carboxyl groups. Carriers can be water soluble.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Synthesis of Stapled Peptide Degron Chimeras

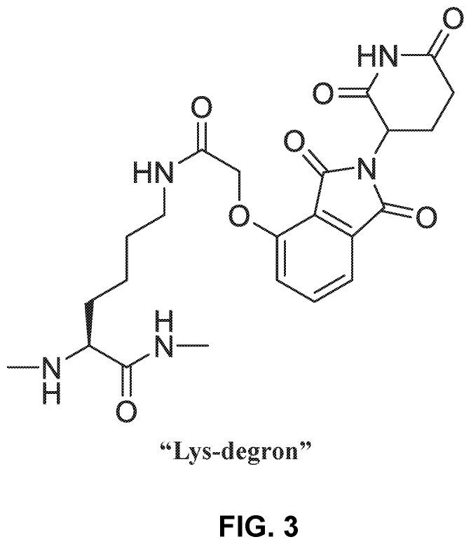

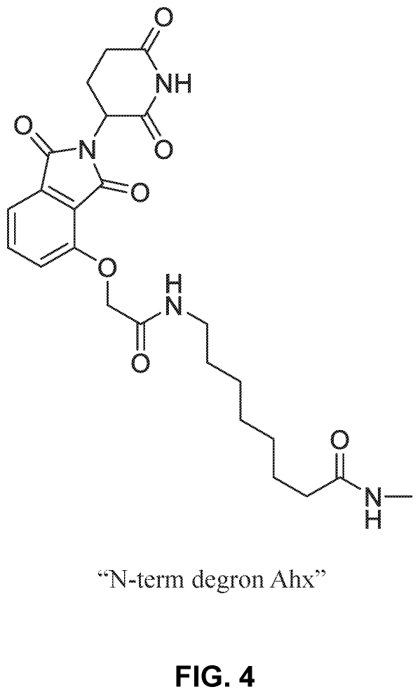

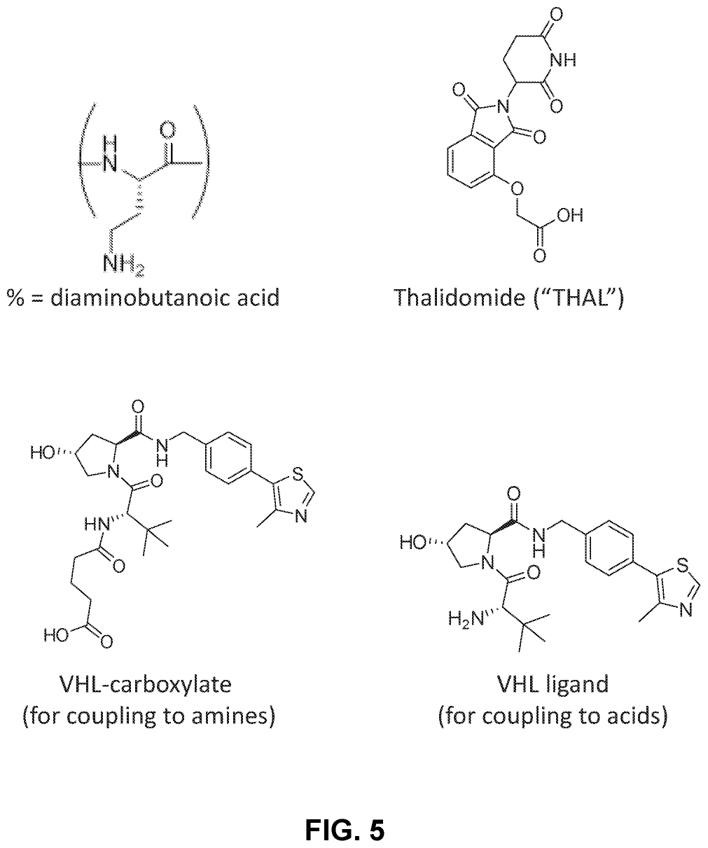

We generated a series of classes of stapled peptide degron chimeras, including: (i) stapled peptide-small molecule degron chimeras, (ii) stapled peptide-peptide degron chimeras, (iii) stapled peptide-stapled peptide degron chimeras, and (iv) small molecule-stapled peptide degron chimeras. The methods for producing each of these classes of stapled peptide degron chimeras are described below. FIGS. 26-29 demonstrate the diversity of approaches in designing the stapled peptide portions of these chimeras. Exemplary components of the chimeras are depicted in FIG. 1-7, 15-16, 21, 23.

Synthesis of Stapled Peptide-Small Molecule Degron Chimeras

Hydrocarbon-stapled peptides were synthesized, purified, and quantitated using previously reported methods (Bird et al., Methods Enzymol., 446:369-86 (2008); Bird et al., Curr. Protoc. Chem. Biol., 3(3):99-117 (2011), with the following modifications and added details: The peptide was synthesized using established methods (i.e., Fmoc protected amino acids, HATU coupling reagent) until the desired sequence was complete. The peptide was then stapled using Grubbs catalyst (generation 1) and the N-terminus deprotected using piperidine. A multi-atom linker was incorporated, such as beta alanine or amino hexanoic acid (see, also, the linkers of FIG. 6). The thalidomide-COOH was then coupled using HCTU. The imide bond is particularly sensitive to nucleophiles; thus, piperidine or hydrazine was not used after incorporation. The peptides were then cleaved for 1 hour with TFA and purified by LCMS.

Synthesis of Stapled Peptide-Peptide Degron Chimeras

The stapled peptide portion of the stapled peptide-peptide degron chimera was synthesized as above (i.e., as for the synthesis of stapled peptide-small molecule degron). The stapled peptide was then coupled to a fully protected peptide degron. The fully-protected degron peptide was synthesized on a weak acid-cleavable resin, specifically, the Sieber amide resin, with the final synthetic step being reaction of glycolic anhydride with the degron peptide N-terminus. After 1%TFA cleavage, the protected degron peptide was precipitated in ether, dissolved in acetic acid/water, and lyophilized. The fully-protected degron peptide was then mixed with coupling reagent and base, and reacted with the resin-bound stapled peptide N-terminus for 2 hours, followed by TFA cleavage and purification to yield the stapled peptide-peptide degron.

Synthesis of Stapled Peptide-Stapled Peptide Degron Chimeras

The first stapled peptide of the stapled peptide-stapled peptide degron chimera was synthesized using the established methods described above (i.e., as for the synthesis of stapled peptide-small molecule degron). A linker moiety, such as beta alanine or amino hexanoic acid, was then incorporated into the first stapled peptide. The second stapled peptide of the chimera was synthesized using the same protocol as the first stapled peptide of the chimera. Next, the entire chimera (i.e., both stapled peptides) was stapled using Grubbs catalyst (generation 1), followed by acetylation of the N-terminus. The chimera was then subjected to cleavage for 1 hour with TFA and purified by LCMS.

Synthesis of Small Molecule-Stapled Peptide Degron Chimeras

The stapled peptide portion of the small molecule-stapled peptide degron chimera was synthesized using the established methods described above (i.e., as for the synthesis of stapled peptide-small molecule degron), followed by peptide stapling using the Grubbs catalyst (generation 1), deprotection of the N-terminus with piperidine, and incorporation of a multi-atom linker, such as beta alanine or amino hexanoic acid.

A small molecule-stapled peptide degron chimera comprising JQ1 as the small molecule was generated. Since the carboxyl group of JQ1 (L. Anders et al., Nat. Biotechnol. 32, 92-96 (2014)) can tolerate chemical substitution, we incubated the resin with JQ1-acid (11.3 mg, 0.0281 mmol, 1 eq) and N-(4-aminobutyl)-2-((2-(2,6-dioxopiperidin-3-yl)-1,3-dioxoisoindolin-4-yl)oxy)acetamidetrifluoroacetate (14.5 mg, 0.0281 mmol, leq) dissolved in DMF (0.28 ml, 0.1 M) at room temperature. DIPEA (14.7 microliters, 0.0843 mmol, 3 eq) and HATU (10.7 mg, 0.0281 mmol, 1 eq) were added, followed by reaction under nitrogen for 20 hours. The small molecule-stapled peptide degron chimera was then subjected to cleavage for 1 hour with TFA and purified by LCMS.

### Example 2: Stapled Peptide Degron Chimeras Retain Target Protein Binding Affinity, and can Achieve Cellular Uptake to Access their Native Targets in Cellulo

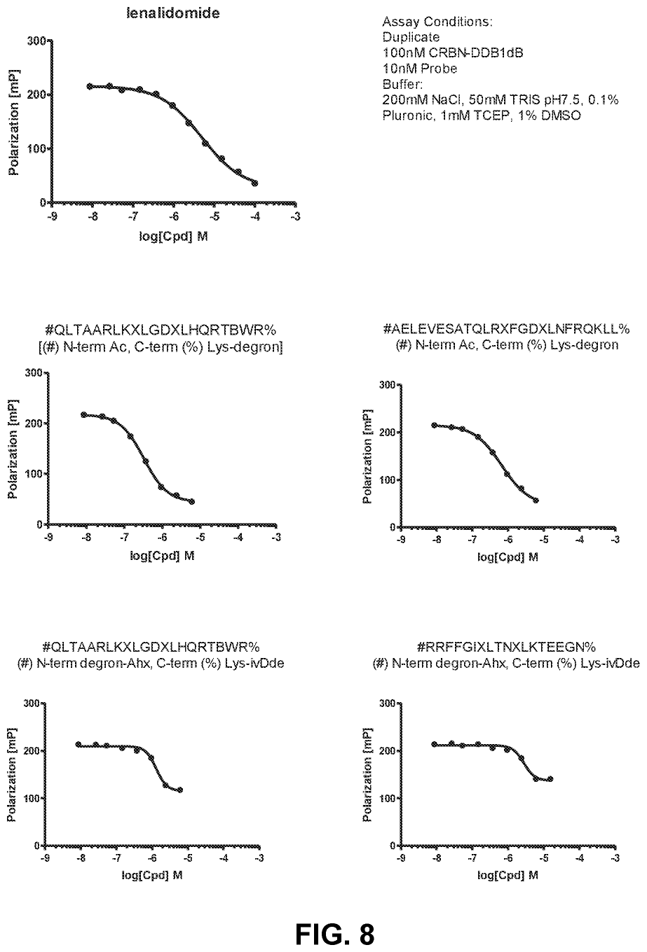

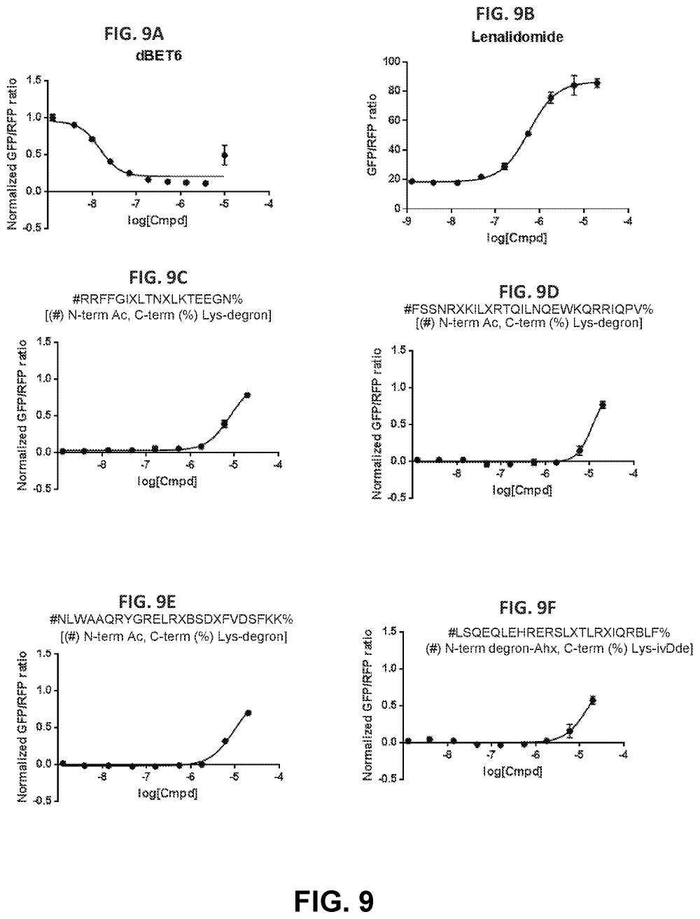

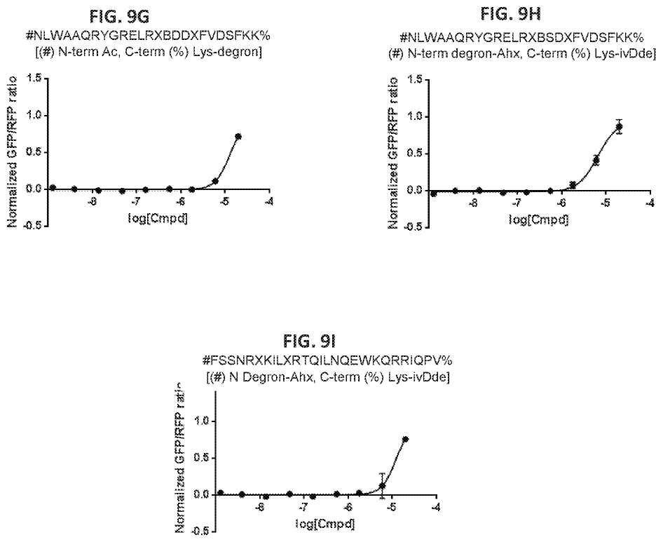

Exemplary fluorescence polarization (FP) binding assays (see Nat Chem Biol. 2018 July; 14(7): 706-714) were performed to demonstrate that a series of stapled peptide degron chimeras incorporating stapled peptides, a linker, and a thalidomide moiety could variably retain binding to cereblon, as monitored by competitive FP (FIG. 8). By use of a cellular assay that involves expression of GFP-BRD4 (exemplary target protein) and administration of dBET6, a small molecule proteolysis targeting chimera (“PROTAC”) that engages Cereblon and BRD4 to induce BRD4 degradation, we further demonstrated that stapled peptide-thalidomide chimeras could enter cells to compete with dBET6 for Cereblon binding and thereby restore GFP-BRD4 (FIG. 9). These data demonstrate that stapled peptide degron chimeras can retain target protein interaction in vitro, gain access to cells, and engage a native protein degrader in cellulo.

### Example 3: Targeted Degradation of an Anti-Apoptotic BCL-2 Family Protein by a Stapled BIM BH3 Peptide Helix-Thalidomide Degron Chimera

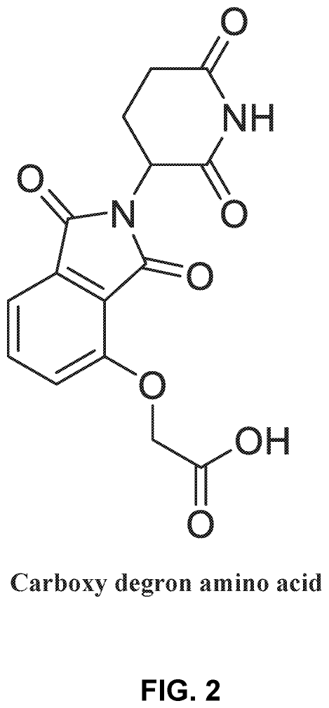

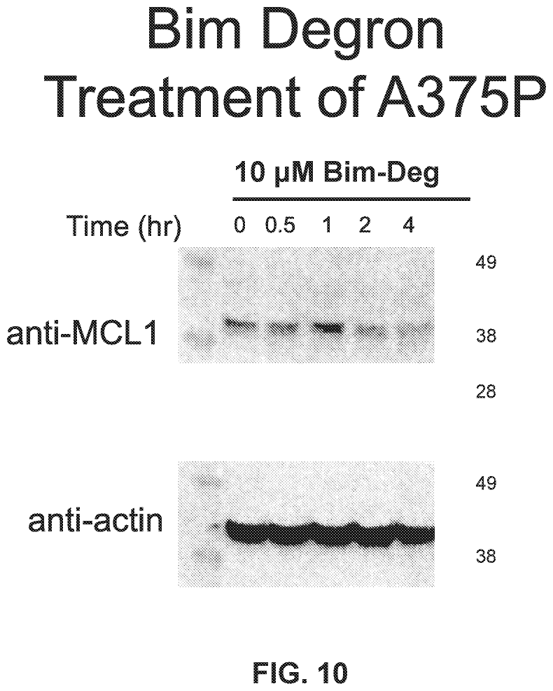

A stapled peptide helix-degron chimeric peptide (FIG. 1) modeled after the pro-apoptotic BIM BH3 domain with an incorporated degron (e.g., Lys-degron, FIG. 2-4) was applied to A375P melanoma cells that express such anti-apoptotic BCL-2 family proteins as MCL-1, which promotes cancer cell survival and chemoresistance. Compound treatment at 10 M followed by monitoring of cellular MCL-1 levels by western blot of lysates revealed a time-dependent decrease in MCL-1 protein as early as the 2 hour time point (FIG. 10). Importantly, the western blotting control for actin protein levels showed no decrement. These data demonstrate that MCL-1 targeting by the BIM BH3 helix, derivatized with a degron moiety, was capable of decreasing the levels of MCL-1 protein in cancer cells within hours of treatment.

### Example 4: Targeted Degradation of the MDM2 Oncoprotein by a Stapled p53 Peptide Helix-Thalidomide Degron Chimera

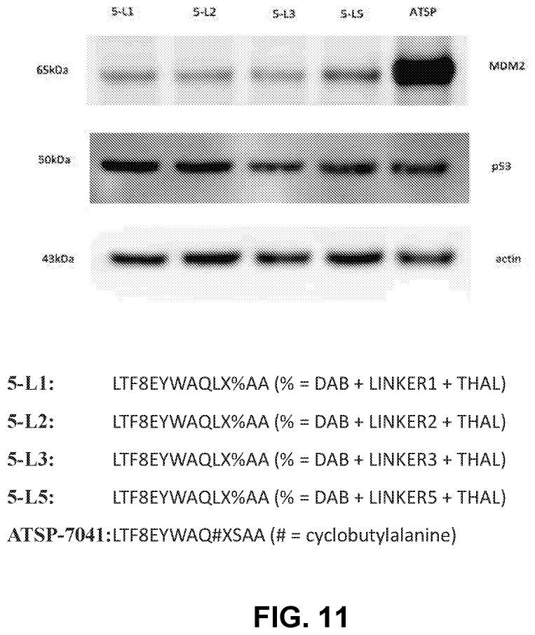

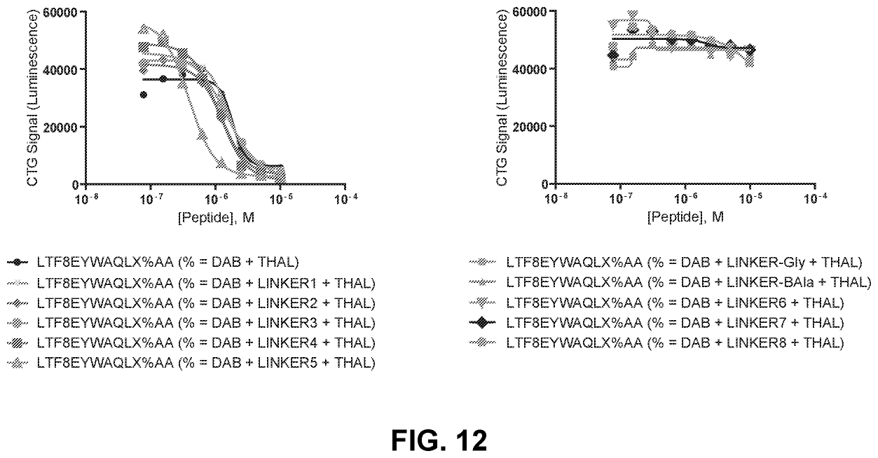

A stapled peptide degron chimera modeled after the transactivation domain helix of p53 (ATSP-7041) coupled to a thalidomide degron was applied to cultured cancer cells (SJSA-1, SJSA-X) and MDM2 protein levels were monitored by western blot and compared to that in cells treated with ATSP-7041 alone. The experiment was repeated and cell viability was measured by Cell Titer Glo assay. The data demonstrate that MDM2 levels were reduced in cells treated with the Thalidomide degron coupled to ATSP-7041 as compared to cells treated with ATSP-7041 alone (FIG. 11). In addition, each of the stapled peptide degron chimeras impaired cancer cell viability in a dose-responsive fashion (FIG. 12). The composition of the linker influenced the presence (FIG. 12, left) or absence (FIG. 12, right) of biological activity.

### Example 5: Cop1-Mediated Protein Degradation

A primary degron is defined as peptide motif that contains a specific sequence pattern that is recognizable by cognate ubiquitin E3 ligases. Primary degrons are usually short, linear motifs within structurally disordered protein regions (Guharoy, M. et al., Nat Commun., 7:10239, doi:10.1038/ncomms10239 (2016)). The primary degron sequence from the protein Trib1 that is recognized by the E3 ligase Cop1 is the amino acid sequence DQIVPEY (SEQ ID NO: 25). In the context of the protein Trib1, the sequence DQIVPEY (SEQ ID NO: 25) confers Trib1 binding to Cop1 such that Trib1 functions as a substrate adaptor and proteins bound to Trib1 are targeted for degradation (Uljon, S. et al., Structure, doi:10.1016/j.str.2016.03.002 (2016)). The reported binding affinity of the sequence DQIVPEY (SEQ ID NO: 25) for Cop1 is 250±40 nM.

We find that the sequence DQIVPEY (SEQ ID NO: 25), and derivatives thereof retaining binding affinity to Cop1, can be used as portable degron sequences causing Cop1-mediated degradation of cellular proteins for therapeutic benefit as described below.

### (1) Direct Genetic Modification:

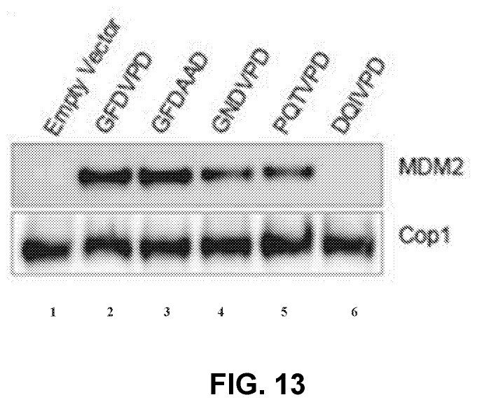

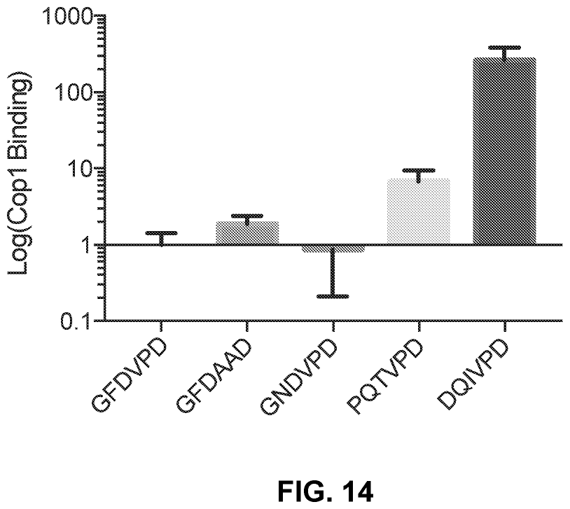

Replacement of natural protein residues in structurally disordered regions with derivatives of the sequence DQIVPEY (SEQ ID NO: 25) can cause the chimera proteins to be degraded by Cop1. For example, replacement of the C-terminal sequence GFDVPD (SEQ ID NO: 26) of the p60 isoform of the protein HDM2 with the Trib1-derived sequence DQIVPD (SEQ ID NO: 30) causes Cop1-mediated degradation of the mutant protein when exogenously expressed in Cop1-expressing human embryonic kidney 293T cells (FIG. 13). Co-immunoprecipitation studies in 293T cells document a correlation between degradation upon specific degron-sequence incorporation (FIG. 13) and direct binding between Cop1 and the corresponding Myc-tagged MDM2 p60 mutant constructs (e.g., DQIVPD (SEQ ID NO:30)) (FIG. 14).

### (2) Peptide Ligand Targeting:

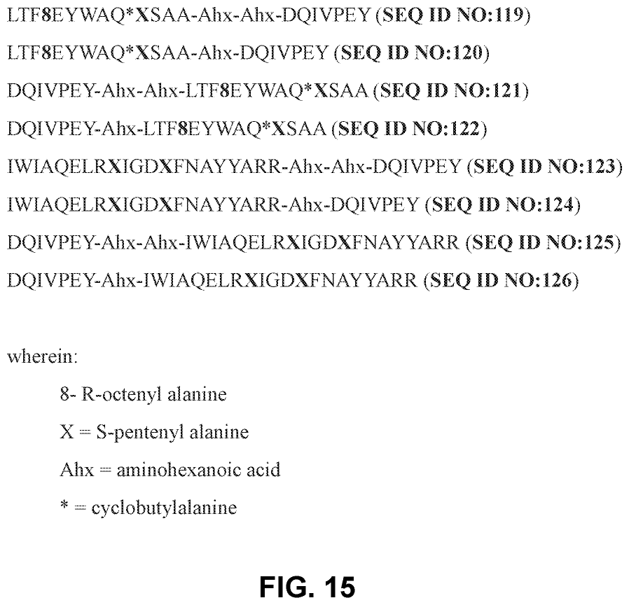

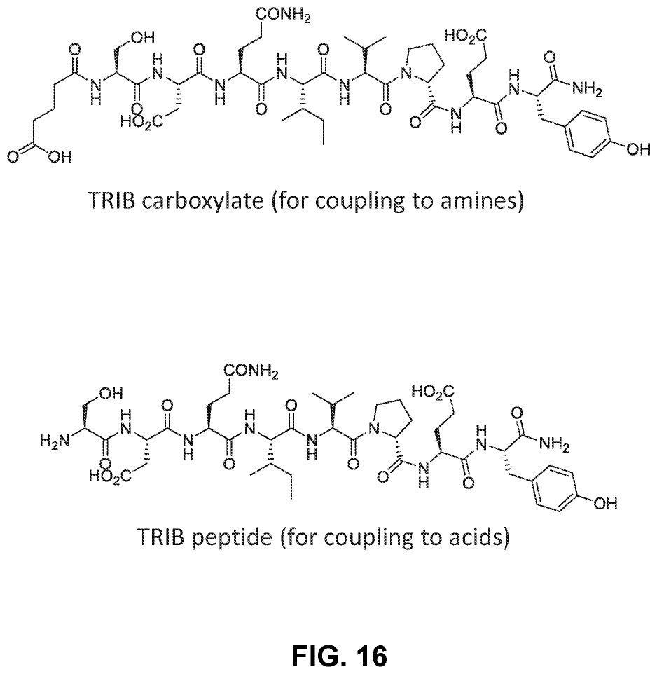

Conjugation of derivatives of the sequence DQIVPEY (SEQ ID NO: 25) to protein-targeting stapled peptides can target the binding partners of the stapled peptides for Cop1-mediated degradation. The design of these conjugates is the same as that outlined in FIG. 1, with the exception that the small molecule thalidomide is replaced with derivatives of the peptide sequence DQIVPEY (SEQ ID NO: 25) and conjugation is achieved through a peptide linker (FIG. 15).

### Example 6: Targeted Degradation of the MDM2 Oncoprotein by a Stapled p53 Peptide Helix-Trib Degron Chimera

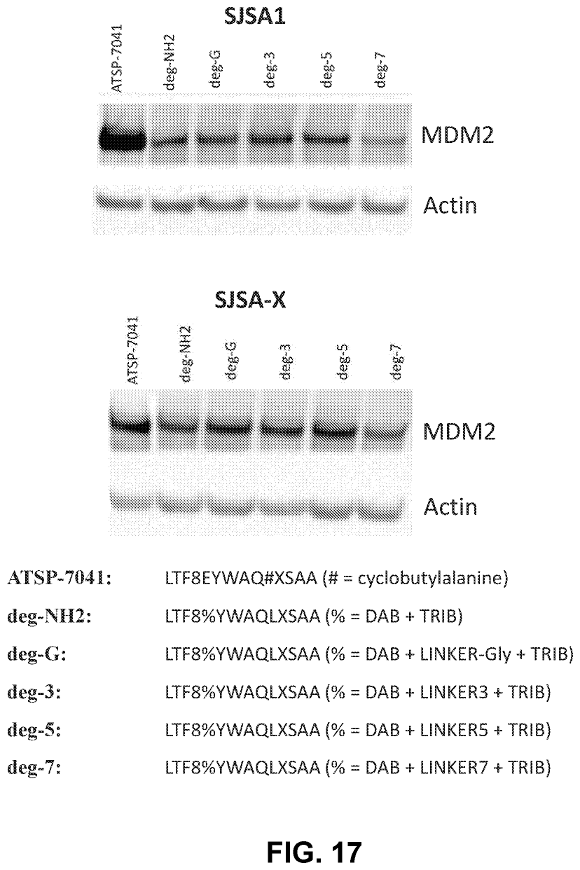

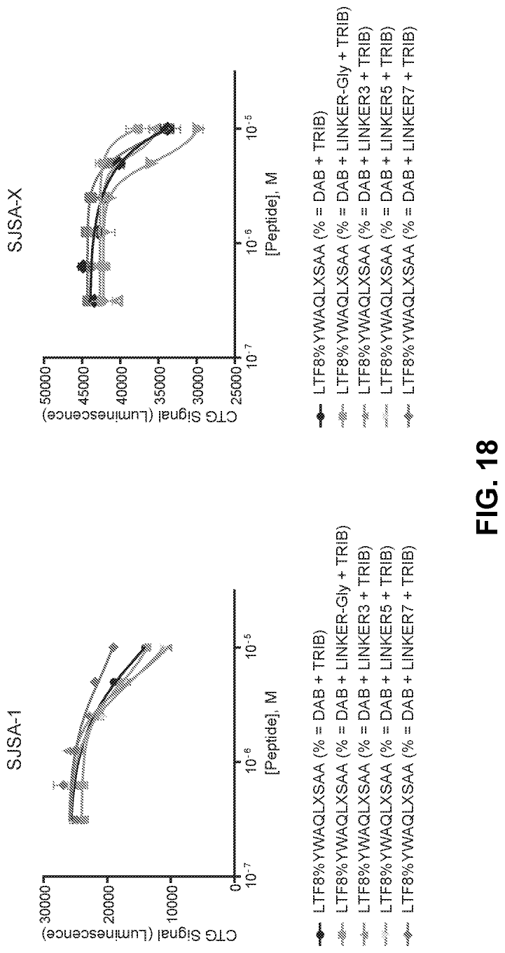

A stapled peptide degron chimera modeled after the transactivation domain helix of p53 (ATSP-7041) coupled to a peptide degron modeled after the sequences from Trib that bind to Cop1 was applied to cultured cancer cells (SJSA-1, SJSA-X) and MDM2 protein levels were monitored by western blot and compared to that in cells treated with ATSP-7041 alone. The experiment was repeated and cell viability was measured by Cell Titer Glo assay. The data demonstrate that MDM2 levels were lower in cells treated with the Trib degron coupled to ATSP-7041 compared to cells treated with ATSP-7041 alone (FIG. 17). In addition, each of the stapled peptide degron chimeras impaired cancer cell viability in a dose-responsive fashion (FIG. 18).

### Example 7: Targeted Degradation of the MDM2 Oncoprotein by a Stapled p53 Peptide Helix-VHL Degron Chimera

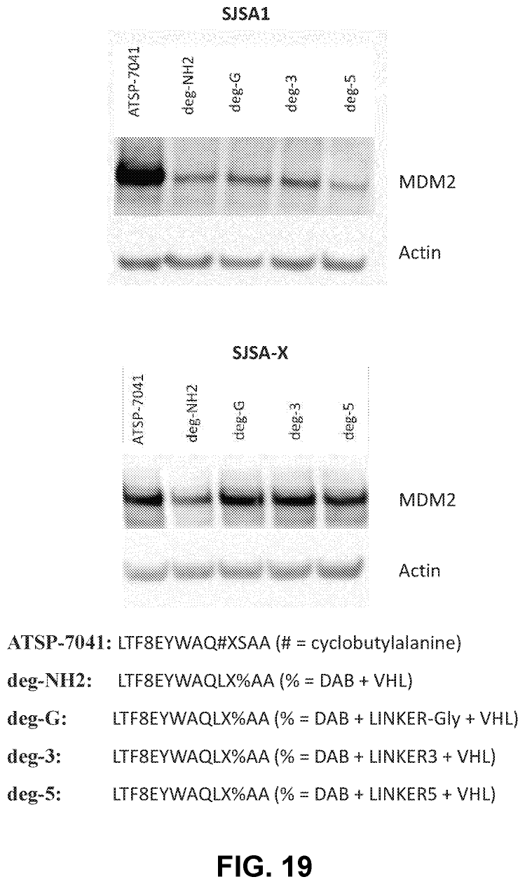

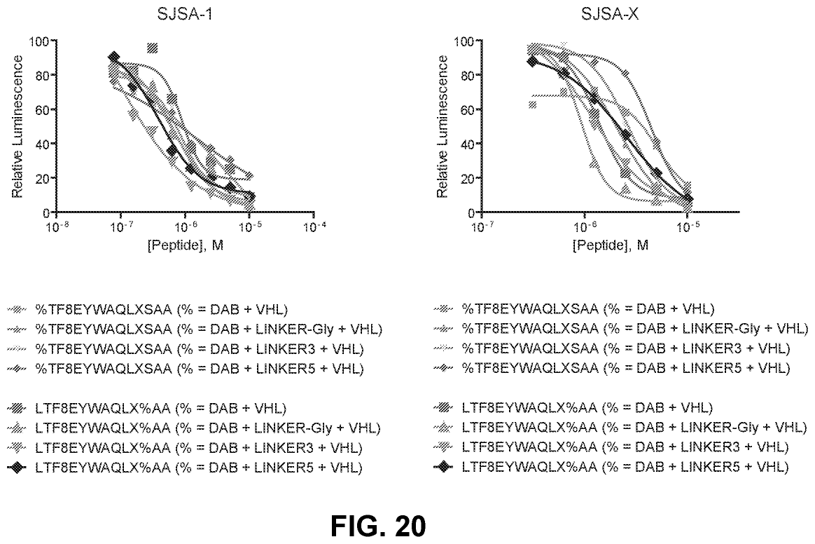

A stapled peptide degron chimera modeled after the transactivation domain helix of p53 (ATSP-7041) coupled to a small molecule degron that binds to VHL was applied to cultured cancer cells (SJSA-1, SJSA-X) and MDM2 protein levels were monitored by western blot and compared to that in cells treated with ATSP-7041 alone. The experiment was repeated and cell viability was measured by Cell Titer Glo assay. The data demonstrate that MDM2 levels were lower in cells treated with the VHL degron coupled to ATSP-7041 compared to cells treated with ATSP-7041 alone (FIG. 19). In addition, each of the stapled peptide degron chimeras impaired cancer cell viability in a dose-responsive fashion (FIG. 20).

### Example 8: Targeted Degradation of the MCL-1 Oncoprotein by a Selective Stapled BH3 Peptide Helix-Stapled p53 Peptide Degron Chimera

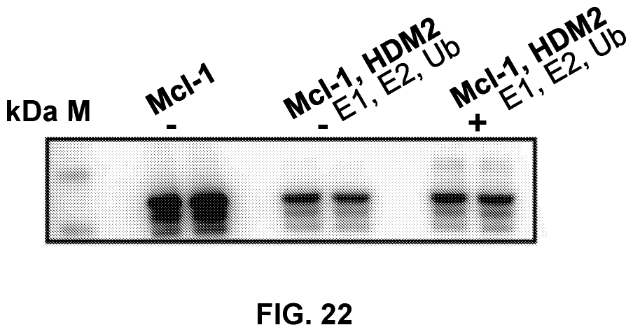

A stapled peptide degron chimera (FIG. 21) modeled after the MCL-1 BH3 helix that selectively targets MCL-1 was coupled to a stapled peptide degron modeled after the transactivation domain helix of p53, with the goal of recruiting MDM2 to MCL-1 to induce non-canonical MDM2-mediated ubiquitination and degradation of MCL-1. Using an in vitro ubiquitination assay (described below), we observed that the addition of the stapled peptide degron chimera to recombinant MCL-1, recombinant MDM2, E1 enzyme, E2 enzyme (UBE2D3) and ATP induced ubiquitination of MCL-1 (FIG. 22). These results show that MDM2 was successfully recruited to MCL-1, only in the presence of the stapled peptide degron chimera, resulting in the transfer of ubiquitin by the ubiquitylation machinery to MCL-1.

### Example 9: Targeted Degradation of the BRD4 Oncoprotein by a Selective Small Molecule BRD4 Inhibitor-Stapled p53 Peptide Degron Chimera

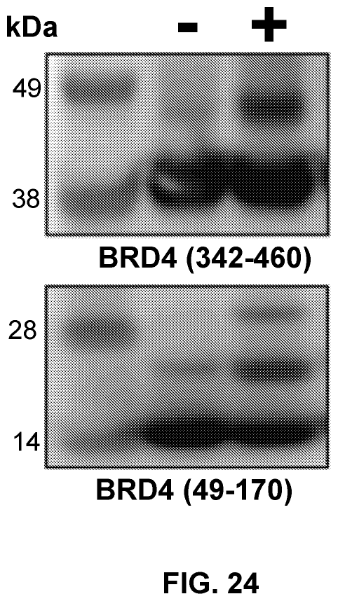

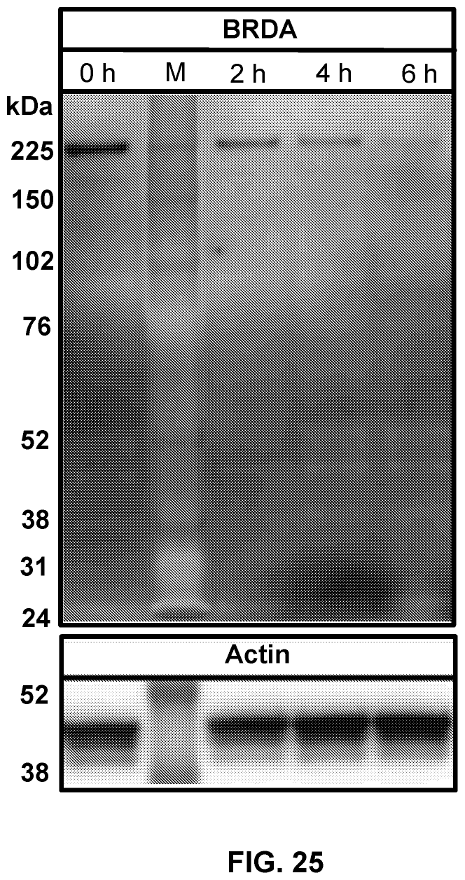

A small molecule that potently targets BRD4 was coupled to a stapled peptide degron modeled after the transactivation domain helix of p53 (FIG. 23), with the goal of recruiting MDM2 to BRD4 to induce non-canonical MDM2-mediated ubiquitination and degradation of BRD4. In a similar manner described above (see Example 8) for evaluating induced ubiquitylation of recombinant MCL-1 protein, we utilized an in vitro assay to test if our stapled peptide degron chimera could recruit MDM2 to ubiquitinate recombinant BRD4 protein (e.g., portions: amino acids 342-460 or amino acids 49-170). Addition of the stapled peptide degron chimera induced upward shifts of each recombinant BRD4 protein, indicating the transfer of ubiquitin to the target BRD4 proteins by MDM2 (FIG. 24). To assess the effect on native BRD4 levels in cancer cells, we treated the U2OS cancer cell line with our stapled peptide degron chimera at 10 μM dosing and observed time-responsive degradation of native BRD4, as reflected by the progressive decrease in BRD4 protein level over time (e.g., 0 to 6 hours) (FIG. 25). These data show that our stapled peptide degron chimera can effectively repurpose MDM2 for ubiquitination of BRD4 in vitro, and induce degradation of native BRD4 in cellulo.

### Materials and Methods

Monitoring Ubiquitination of Recombinant Protein Targets as Induced by Stapled Peptide Degron Chimeras.

To monitor ubiquitination of protein targets in vitro, a commercial Mdm2/HDM2 Ubiquitin Ligase Kit (K-200B) was employed. Briefly, chimera (10 μM), recombinant full length MDM2 (GST-tagged, 1 μM), E1 enzyme (UBE1, 50 nM), E2 enzyme (UBE2D3, 1 μM), ubiquitin (100 μM), ATP (1 mM) and recombinant target protein (100 nM) were combined in a 1.5 mL microtube in reaction buffer. The mixture was incubated at 37 degrees Celsius for six hours. Subsequently, 20 uL of reaction mixture was assayed using standard western blotting techniques whereby antibodies raised against the target protein were used to visualize the upward band shifts due to ubiquitination.

Monitoring Native Protein Degradation in Cellulo as Induced by Stapled Peptide Degron Chimeras.

To assay for intracellular protein degradation, cancer cells (e.g. SJSA-1, SJSA-X, U20S) were passaged at 37 degrees Celsius in a humidity-controlled, C₂-equilibrated incubator in DMEM (Life Technologies, Grand Island, N.Y.) culture medium (CM) containing 10% fetal bovine serum (FBS) and 1% penicillin/streptomycin (Pen Strep). One day before treatment, cells were passaged and plated in six well plates at a density of 100,000 cells/mL. After 24 hours, cells were treated with a stapled peptide degron chimera (e.g., 10 μM) for 0, 2, 4 and 6 hours after which they were harvested and lysed. Cell lysates were then assayed using standard western blotting techniques using an antibody raised against the target protein to assess protein level and an actin antibody for the loading control.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A chimera, comprising:
a first moiety attached to a second moiety;
wherein the first moiety binds to a first protein targeted for degradation; and
the second moiety binds to a second protein, wherein the second protein is a protein degrader.

2. The chimera of claim 1, wherein the first moiety and second moiety are covalently attached to each other.

3. The chimera of claim 1, wherein the first moiety and the second moiety are attached to each other via a linker.

4. The chimera of any one of claims 1 to 3, wherein the first protein is a disease-causing or disease-related protein.

5. The chimera of any one of claims 1 to 4, wherein the first protein targeted for degradation is a killer protein, a protein that is damaging to cells, a protein that causes neurodegeneration, BCL2, BCLXL, MCL-1, BFL-1, BCL-w, BCL-B, EZH2, HDM2/HDMX, KRAS/NRAS/HRAS, MYC, β-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, CCR5, a bacterial protein, or a viral protein.

6. The chimera of any one of claims 1 to 5, wherein the first moiety comprises a first stapled peptide that binds to the first protein targeted for degradation.

7. The chimera of claim 6, wherein the first stapled peptide does not comprise a Bcl-2 homology 3 (BH3) domain polypeptide.

8. The chimera of claim 6, wherein the first stapled peptide does not comprise: (a) a Bcl-2 homology 3 domain from MCL-1, (b) a MCL-1 stabilized alpha helix of BCL2 domain, or (c) MCL-1 SAHBD.

9. The chimera of claim any one of claims 6 to 8, wherein the second moiety is attached to the N-terminus of the first moiety.

10. The chimera of claim any one of claims 6 to 8, wherein the second moiety is attached to the C-terminus of the first moiety.

11. The chimera of claim any one of claims 6 to 8, wherein the second moiety is attached to an internal amino acid position of the first moiety.

12. The chimera of any one of claims 1 to 11, wherein the first moiety comprises a small molecule that binds to the first protein targeted for degradation.

13. The chimera of any one of claims 1 to 11, wherein the second moiety comprises a peptide degron that binds to the protein degrader.

14. The chimera of any one of claims 1 to 12, wherein the second moiety comprises a second stapled peptide that binds to the protein degrader.

15. The chimera of any one of claims 1 to 11, wherein the second moiety comprises a small molecule that binds to the protein degrader.

16. The chimera of any one of claims 1 to 8, wherein the second moiety comprises a second stapled peptide that binds to the protein degrader, and wherein the first moiety is attached to the N-terminus of the second moiety.

17. The chimera of any one of claims 1 to 8, wherein the second moiety comprises a second stapled peptide that binds to the protein degrader, and wherein the first moiety is attached to the C-terminus of the second moiety.

18. The chimera of any one of claims 1 to 8, wherein the second moiety comprises a second stapled peptide that binds to the protein degrader, and wherein the first moiety is attached to an internal amino acid position of the second moiety.

19. The chimera of any one of claims 1 to 18, wherein the protein degrader degrades the first protein targeted for degradation.

20. A method of treating a disease or disorder driven by a pathologic peptide or protein in a subject in need thereof, the method comprising administering to the subject a therapeutically effective amount of the chimera of any one of claims 1 to 19.

21. A chimeric polypeptide comprising a stapled peptide and a peptide that binds a WD40-repeat protein that is a substrate adaptor for an E3 ubiquitin ligase, wherein the peptide comprises a modified version of a natural binding sequence or a natural binding consensus sequence of an amino acid sequence that binds to the WD40-repeat protein, wherein the modified version comprises at least one amino acid substitution, at least one amino acid deletion, at least one amino acid insertion, or any combination thereof within the natural binding consensus sequence.

22. The chimeric polypeptide of claim 21, wherein the WD40-repeat protein that is a substrate adaptor for an E3 ubiquitin ligase is selected from the group consisting of MDM2, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1.

23. The chimeric polypeptide of claim 21, wherein the natural binding consensus sequence is a sequence selected from the group consisting of SEQ ID NOs.: 25, 31-46, and 65-105.

24. The chimeric polypeptide of claim 21, wherein the natural binding sequence is SEQ ID NO:25.

25. The chimeric polypeptide of claim 21, wherein the natural binding consensus sequence is SEQ ID NO:46.

26. The chimeric polypeptide of claim 24, wherein the peptide comprises the amino acid sequence set forth in SEQ ID NO:25 except having at least one amino acid substitution.

27. The chimeric polypeptide of claim 24, wherein the peptide comprises the amino acid sequence set forth in SEQ ID NO:25 except having at least one amino acid deletion.

28. The chimeric polypeptide of claim 24, wherein the peptide comprises the amino acid sequence set forth in SEQ ID NO:25 except having at least one amino acid substitution and at least one amino acid deletion.

29. The chimeric polypeptide of claim 24, wherein the peptide comprises the amino acid sequence set forth in SEQ ID NO:25 except having one to six amino acid substitutions.

30. The chimeric polypeptide of claim 24, wherein position 4(V) and position 5 (P) of SEQ ID NO:25 are not substituted.

31. The chimeric polypeptide of claim 24, wherein one or more of positions 1(D), position 2 (Q), position 3 (I), and position 6 (E) of SEQ ID NO:25 are substituted.

32. The chimeric polypeptide of claim 24, wherein the peptide comprises the amino acid sequence set forth in SEQ ID NO:25 except having one amino acid deletion.

33. The chimeric polypeptide of claim 24, wherein position 7 (Y) of the amino acid sequence set forth in SEQ ID NO:25 is deleted.

34. The chimeric polypeptide of claim 24, wherein the peptide comprises the amino acid sequence set forth in SEQ ID NO:25 except having with one to six amino acid substitutions and at least one amino acid deletion.

35. The chimeric polypeptide of claim 24, wherein the peptide has an amino acid sequence selected from the group consisting of SEQ ID NOs.: 26 to 30.

36. The chimeric polypeptide of claim 24, wherein the peptide has the amino acid sequence set forth in SEQ ID NO:30.

37. The chimeric polypeptide of any one of claims 24 to 36, wherein the peptide is 4 to 30 amino acids in length.

38. The chimeric polypeptide of any one of claims 24 and 25 to 37, wherein the peptide binds Cop1 with a binding affinity of:
1 nM to 300 nM;
10 nM to 300 nM;
100 nM to 300 nM; or
200 nM to 300 nM.

39. The chimeric polypeptide of any one of claims 24 to 38, wherein the stapled peptide targets an intracellular protein, extracellular protein, or cell surface protein.

40. The chimeric polypeptide of any one of claims 24 to 38, wherein the stapled peptide targets a disease-causing or disease-related protein.

41. The chimeric polypeptide of any one of claims 24 to 38, wherein the stapled peptide targets a killer protein (e.g., BAX, BAK) or a protein that is damaging to cells that causes neurodegeneration (e.g., IgG, beta-amyloid, tau, α-synuclein, TDP-43, HbS, superoxide dismutase, Notch3, FUS, GFAP).

42. The chimeric polypeptide of any one of claims 24 to 28, wherein the stapled peptide targets a protein selected from the group consisting of BCL2, BCLXL, MCL-1, BFL-1, BCL-w, BCL-B, EZH2, HDM2/HDMX, KRAS/NRAS/HRAS, MYC, β-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5.

43. The chimeric polypeptide of any one of claims 24 to 28, wherein the stapled peptide targets a bacterial protein.

44. The chimeric polypeptide of any one of claims 24 to 38, wherein the stapled peptide targets a viral protein.

45. A modified protein of a first protein that comprises a structurally disordered region, wherein the modified protein differs from the first protein in that the structurally disordered region comprises a peptide that binds a WD40-repeat protein that is a substrate adaptor for an E3 ubiquitin ligase, the peptide comprising a modified version of a natural binding sequence or a natural binding consensus sequence, wherein the modified version comprises at least one amino acid substitution, at least one amino acid deletion, at least one amino acid insertion, or any combination thereof within the natural binding consensus sequence.

46. The modified protein of claim 45, wherein the WD40-repeat protein that is a substrate adaptor for an E3 ubiquitin ligase is selected from the group consisting of MDM2, SKP2-CKS1, FBXW1, FBXW2, FBXW4, FBXW5, FBXW7, FBXW8, FBXW9, FBXW10, FBXW11, FBXW12, SPOP, VHL, ITCH, KEAP1, KLHL2, KLHL3, KLHL7, KLHL12, KLHL13, KLHL15, KLHL20, KLHL21, KLHL24, KLHL40, KLHL42, COP1, TRAF7, RFWD3, DCAF1, DCAF2, DCAF3, DCAF4, DCAF5, DCAF6, DCAF7, DCAF8, DCAF9, DCAF10, DCAF11, DCAF12, DCAF13, DCAF14, DCAF15, DCAF16, DCAF17, DCAF19, SIAH1, TRPC4AC, DET1, WSB1, WSB2, HERC1, DDB2, CSA, CBL, CDC20, and FZR1.

47. The modified protein of claim 45, wherein the natural binding sequence or the natural binding consensus sequence is a sequence selected from the group consisting of SEQ ID NOs.: 25, 31-46, and 65-105.

48. The modified protein of claim 45, wherein the natural binding sequence is SEQ ID NO:25.

49. The modified protein of claim 45, wherein the natural binding consensus sequence is SEQ ID NO:46.

50. The modified protein of claim 48, wherein the peptide comprises the amino acid sequence set forth in SEQ ID NO:25 except having at least one amino acid substitution.

51. The modified protein of claim 48, wherein the peptide comprises the amino acid sequence set forth in SEQ ID NO:25 except having at least one amino acid deletion.

52. The modified protein of claim 48, wherein the peptide comprises the amino acid sequence set forth in SEQ ID NO:25 except having at least one amino acid substitution and at least one amino acid deletion.

53. The modified protein of claim 48, wherein the peptide comprises the amino acid sequence set forth in SEQ ID NO:25 except having one to six amino acid substitutions.

54. The modified protein of claim 48, wherein position 4(V) and position 5 (P) of SEQ ID NO:25 are not substituted.

55. The modified protein of claim 48, wherein one or more of positions 1(D), position 2 (Q), position 3 (I), and position 6 (E) of SEQ ID NO:25 are substituted.

56. The modified protein of claim 48, wherein the peptide comprises the amino acid sequence set forth in SEQ ID NO:25 except having one amino acid deletion.

57. The modified protein of claim 48, wherein position 7 (Y) of the amino acid sequence set forth in SEQ ID NO:25 is deleted.

58. The peptide of claim 48, wherein the peptide comprises the amino acid sequence set forth in SEQ ID NO:25 except having with one to six amino acid substitutions and at least one amino acid deletion.

59. The peptide of claim 48, wherein the peptide has an amino acid sequence selected from the group consisting of SEQ ID NOs.: 26 to 30.

60. The peptide of claim 48, wherein the peptide has the amino acid sequence set forth in SEQ ID NO:30.

61. The peptide of any one of claims 45 to 60, wherein the peptide is 4 to 10 amino acids in length.

62. The peptide of any one of claims 48, or 50 to 61, wherein the peptide binds Cop1 with a binding affinity of:
1 nM to 300 nM;
10 nM to 300 nM;
100 nM to 300 nM; or
200 nM to 300 nM.

63. A method of treating a disease or disorder driven by a pathologic peptide or protein in a subject in need thereof, the method comprising administering to the subject a therapeutically effective amount of the chimeric fusion polypeptide of any one of claims 21 to 44.

64. A peptide small molecule fusion comprising a protein-targeting stapled peptide and a thalidomide degron moiety.

65. The peptide small molecule fusion of claim 64, wherein the thalidomide moiety is conjugated to the N-terminus of the protein-targeting stapled peptide.

66. The peptide small molecule fusion of claim 64, wherein the thalidomide moiety is conjugated to the C-terminus of the protein-targeting stapled peptide.

67. The peptide small molecule fusion of claim 64, wherein the thalidomide moiety is contained within a non-natural amino acid inserted in the peptide sequence between the N- and C-terminus of the protein-targeting stapled peptide.

68. The peptide small molecule fusion of any one of claims 64 to 67, wherein the stapled peptide targets a disease-causing protein.

69. The peptide small molecule fusion of any one of claims 64 to 67, wherein the stapled peptide targets an intracellular protein, receptor protein, or an extracellular protein.

70. The peptide small molecule fusion of any one of claims 64 to 67, wherein the stapled peptide targets a killer protein (e.g., BAX, BAK) or a protein that is damaging to cells that causes neurodegeneration (e.g., IgG, beta-amyloid, tau, α-synuclein, TDP-43, HbS, superoxide dismutase, Notch3, FUS, GFAP).

71. The peptide small molecule fusion of claim 69, wherein the intracellular protein, receptor protein, or an extracellular protein is selected from the group consisting of BCL2, BCLXL, MCL-1, BFL-1, BCL-w, BCL-B, EZH2, HDM2/HDMX, KRAS/NRAS/HRAS, MYC, b-catenin, PI3K, PTEN, TSC, AKT, BRCA1/2, EWS-FLI, MLL fusions, a receptor Tyrosine kinases, a HOX homolog, JUN, Cyclin D, Cyclin E, BRAF, CRAF, CDK4, CDK2, HPV-E6/E7, Aurora kinase, MITF, Wnt1, PD-1, BCR, and CCR5.

72. The peptide small molecule fusion of any one of claims 64 to 67, wherein the stapled peptide targets a bacterial protein.

73. The peptide small molecule fusion of any one of claims 64 to 67, wherein the stapled peptide targets a viral protein.

74. The peptide small molecule fusion of any one of claims 64 to 73, wherein the thalidomide moiety comprises the structure provided below:

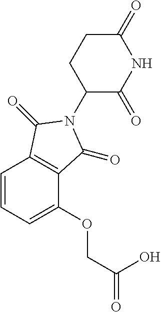 [SMILES: O=C(O)COc1cccc2c1C(=O)N(C1CCC(=O)NC1=O)C2=O]

75. The peptide small molecule fusion of any one of claims 64 and 67 to 73, wherein the thalidomide moiety comprises the structure provided below:

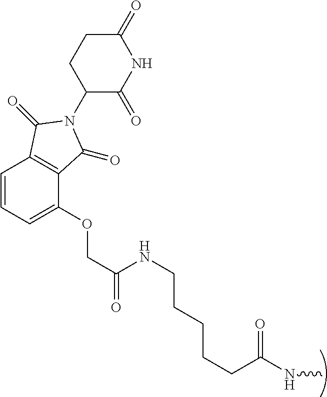

76. The peptide small molecule fusion of any one of claims 68 to 73, wherein the thalidomide moiety comprises the structure provided below:

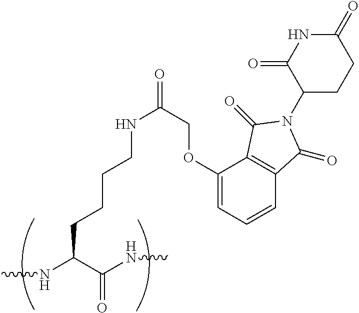

77. A method of treating a disease or disorder driven by a pathologic peptide or protein in a subject in need thereof, the method comprising administering to the subject a therapeutically effective amount of the peptide small molecule fusion of any one of claims 64 to 76.

78. A peptide small molecule fusion comprising a protein-targeting stapled peptide and a Von Hippel-Lindau (VHL) degron moiety.

79. The peptide small molecule fusion of claim 78, wherein the VHL degron moiety is conjugated to the N-terminus of the protein-targeting stapled peptide.

80. The peptide small molecule fusion of claim 79, wherein the VHL degron moiety comprises the structure below:

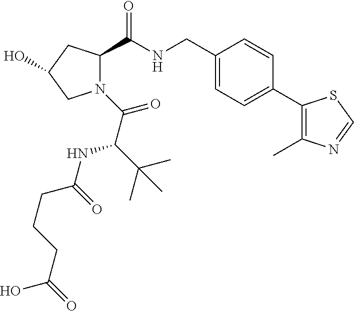 [SMILES: Cc1ncsc1-c1ccc(CNC(=O)[C@@H]2C[C@@H](O)CN2C(=O)[C@@H](NC(=O)CCCC(=O)O)C(C)(C)C)cc1]

81. The peptide small molecule fusion of claim 78, wherein the VHL degron moiety is conjugated to the C-terminus of the protein-targeting stapled peptide.

82. The peptide small molecule of claim 81, wherein the VHL degron moiety comprises the structure below:

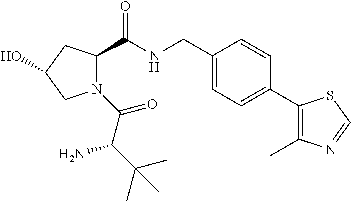 [SMILES: Cc1ncsc1-c1ccc(CNC(=O)[C@@H]2C[C@@H](O)CN2C(=O)[C@@H](N)C(C)(C)C)cc1]

83. The peptide small molecule fusion of claim 78, wherein the thalidomide moiety is contained within a non-natural amino acid inserted in the peptide sequence between the N- and C-terminus of the protein-targeting stapled peptide.

84. The peptide small molecule fusion of any one of claims 78 to 83, wherein the stapled peptide targets a disease-causing protein.

85. The peptide small molecule fusion of any one of claims 78 to 83, wherein the stapled peptide targets an intracellular protein, receptor protein, or an extracellular protein.

86. The peptide small molecule fusion of any one of claims 78 to 84, wherein the stapled peptide targets a killer protein (e.g., BAX, BAK) or a protein that is damaging to cells that causes neurodegeneration (e.g., IgG, beta-amyloid, tau, α-synuclein, TDP-43, HbS, superoxide dismutase, Notch3, FUS, GFAP).

87. The peptide small molecule fusion of claim 85, wherein the intracellular protein, receptor protein, or an extracellular protein is selected from the group consisting of BCL2, BCLXL, MCL-1, BFL-1, BCL-w, BCL-B, EZH2, HDM2/HDMX, KRAS/NRAS/HRAS, MYC, b-catenin, PI3K, PTEN, TSC, AKT, BRCA1/2, EWS-FLI, MLL fusions, a receptor Tyrosine kinases, a HOX homolog, JUN, Cyclin D, Cyclin E, BRAF, CRAF, CDK4, CDK2, HPV-E6/E7, Aurora kinase, MITF, Wnt1, PD-1, BCR, and CCR5.

88. The peptide small molecule fusion of any one of claims 78 to 83, wherein the stapled peptide targets a bacterial protein.

89. The peptide small molecule fusion of any one of claims 78 to 83, wherein the stapled peptide targets a viral protein.

90. A method of treating a disease or disorder driven by a pathologic peptide or protein in a subject in need thereof, the method comprising administering to the subject a therapeutically effective amount of the peptide small molecule fusion of any one of claims 78 to 89.

91. A peptide that binds Constitutive Photomorphogenic 1 (Cop1) protein, wherein the peptide comprises a modified version of the amino acid sequence DQIVPEY (SEQ ID NO:25), wherein the modified version comprises at least one amino acid substitution, at least one amino acid deletion, at least one amino acid insertion, or any combination thereof in SEQ ID NO:25, but wherein if the modified version consists of a single amino acid substitution, then the amino acid substitution is not to A or R at any one of positions 1 to 7 of SEQ ID NO:25, or to V at position 4 of SEQ ID NO:25.

92. The peptide of claim 91, wherein the peptide comprises the amino acid sequence set forth in SEQ ID NO:25 except having at least one amino acid substitution.

93. The peptide of claim 91, wherein the peptide comprises the amino acid sequence set forth in SEQ ID NO:25 except having at least one amino acid deletion.

94. The peptide of claim 91, wherein the peptide comprises the amino acid sequence set forth in SEQ ID NO:25 except having at least one amino acid substitution and at least one amino acid deletion.

95. The peptide of claim 91, wherein the peptide comprises the amino acid sequence set forth in SEQ ID NO:25 except having one to six amino acid substitutions.

96. The peptide of claim 91, wherein position 4(V) and/or position 5 (P) of SEQ ID NO:25 are not substituted.

97. The peptide of claim 91, wherein one or more of positions 1(D), position 2(Q), position 3 (I), and position 6 (E) of SEQ ID NO:25 are substituted.

98. The peptide of claim 91, wherein the peptide comprises the amino acid sequence set forth in SEQ ID NO:25 except having one amino acid deletion.

99. The peptide of claim 91, wherein position 7 (Y) of the amino acid sequence set forth in SEQ ID NO:25 is deleted.

100. The peptide of claim 91, wherein the peptide comprises the amino acid sequence set forth in SEQ ID NO:25 except having with one to six amino acid substitutions and at least one amino acid deletion.

101. The peptide of claim 91, wherein the peptide has an amino acid sequence selected from the group consisting of SEQ ID NOs.: 26 to 30.

102. The peptide of claim 91, wherein the peptide has the amino acid sequence set forth in SEQ ID NO:30.

103. The peptide of any one of claims 91 to 102, wherein the peptide is 4 to 10 amino acids in length.

104. The peptide of any one of claims 91 to 102, wherein the peptide binds Cop1 with a binding affinity of 1 nM to 300 nM.

105. The peptide of any one of claims 91 to 102, wherein the peptide binds Cop1 with a binding affinity of 10 nM to 300 nM.

106. The peptide of any one of claims 91 to 102, wherein the peptide binds Cop1 with a binding affinity of 100 nM to 300 nM.

107. The peptide of any one of claims 91 to 102, wherein the peptide binds Cop1 with a binding affinity of 200 nM to 300 nM.

108. A chimeric fusion polypeptide comprising a protein-targeting stapled peptide and a peptide of any one of claims 91 to 107.

109. The chimeric fusion polypeptide of claim 108, wherein the stapled peptide targets an intracellular protein or a cell surface receptor.

110. The chimeric fusion polypeptide of claim 108, wherein the stapled peptide targets a disease-causing or disease-related protein.

111. The chimeric fusion polypeptide of claim 108, wherein the stapled peptide targets a killer protein (e.g., BAX, BAK) or a protein that is damaging to cells that causes neurodegeneration (e.g., IgG, beta-amyloid, tau, α-synuclein, TDP-43, HbS, superoxide dismutase, Notch3, FUS, GFAP).

112. The chimeric fusion polypeptide of claim 109, wherein the intracellular protein or cell surface receptor is selected from the group consisting of BCL2, BCLXL, MCL-1, BFL-1, BCL-w, BCL-B, EZH2, HDM2/HDMX, KRAS/NRAS/HRAS, MYC, β-catenin, PI3K, PTEN, TSC, AKT, BRCA1/2, a EWS-FLI fusion, an MLL fusion, a receptor tyrosine kinase, a HOX homolog, JUN, Cyclin D, Cyclin E, BRAF, CRAF, CDK4, CDK2, HPV-E6/E7, Aurora kinase, MITF, Wnt1, PD-1, BCR, and CCR5.

113. The chimeric fusion polypeptide of claim 108, wherein the stapled peptide targets a bacterial protein.

114. The chimeric fusion polypeptide of claim 108, wherein the stapled peptide targets a viral protein.

115. A method of treating a disease or disorder driven by a pathologic peptide or protein in a subject in need thereof, the method comprising administering to the subject a therapeutically effective amount of the chimeric fusion polypeptide of any one of claims 108 to 114.


SEQUENCE LISTING (145 sequences)

SEQ ID NO: 1 - protein, 26 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“ ”
  MOD_RES (13)..(13): S-pentenyl alanine or any amino acid
  MOD_RES (14)..(14): Norleucine
  MOD_RES (17)..(17): S-pentenyl alanine or any amino acid
  MOD_RES (26)..(26): Lys-degron
  VARIANT (26)..(26): /replace=“ ”
  SITE (1)..(26): /note=“Variant residues given in the sequence have no preference with respect to those in the annotations for variant positions”
  source: /note=“See specification as filed for detailed description of substitutions and preferred embodiments”
XQWAREIGAQLRXXADXLNAQYERRX

SEQ ID NO: 2 - protein, 29 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“Degron-Ahx” or “ ”
  MOD_RES (7)..(7): S-pentenyl alanine or any amino acid
  MOD_RES (11)..(11): S-pentenyl alanine or any amino acid
  MOD_RES (29)..(29): Lys-degron
  VARIANT (29)..(29): /replace=“Lys-ivDde” or “ ”
  SITE (1)..(29): /note=“Variant residues given in the sequence have no preference with respect to those in the annotations for variant positions”
  source: /note=“See specification as filed for detailed description of substitutions and preferred embodiments”
XFSSNRXKILXRTQILNQEWKQRRIQPVX

SEQ ID NO: 3 - protein, 20 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“Degron-Ahx” or “ ”
  MOD_RES (8)..(8): S-pentenyl alanine or any amino acid
  MOD_RES (12)..(12): S-pentenyl alanine or any amino acid
  MOD_RES (20)..(20): Lys-degron
  VARIANT (20)..(20): /replace=“Lys-ivDde” or “ ”
  SITE (1)..(20): /note=“Variant residues given in the sequence have no preference with respect to those in the annotations for variant positions”
  source: /note=“See specification as filed for detailed description of substitutions and preferred embodiments”
XRRFFGIXLTNXLKTEEGNX

SEQ ID NO: 4 - protein, 24 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“ ”
  MOD_RES (16)..(16): S-pentenyl alanine or any amino acid
  MOD_RES (20)..(20): S-pentenyl alanine or any amino acid
  MOD_RES (24)..(24): Lys-degron
  VARIANT (24)..(24): /replace=“ ”
  SITE (1)..(24): /note=“Variant residues given in the sequence have no preference with respect to those in the annotations for variant positions”
  source: /note=“See specification as filed for detailed description of substitutions and preferred embodiments”
XRKALETLRRVGDGVXRNHXTAFX

SEQ ID NO: 5 - protein, 26 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“Degron-Ahx” or “ ”
  MOD_RES (15)..(15): S-pentenyl alanine or any amino acid
  MOD_RES (19)..(19): S-pentenyl alanine or any amino acid
  MOD_RES (23)..(23): Norleucine
  MOD_RES (26)..(26): Lys-degron
  VARIANT (26)..(26): /replace=“Lys-ivDde” or “ ”
  SITE (1)..(26): /note=“Variant residues given in the sequence have no preference with respect to those in the annotations for variant positions”
  source: /note=“See specification as filed for detailed description of substitutions and preferred embodiments”
XLSQEQLEHRERSLXTLRXIQRXLFX

SEQ ID NO: 6 - protein, 16 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“ ”
  MOD_RES (5)..(5): R-octenyl alanine
  MOD_RES (11)..(11): Cyclobutylalanine
  MOD_RES (12)..(12): S-pentenyl alanine
  MOD_RES (16)..(16): Lys-degron
  VARIANT (16)..(16): /replace=“ ”
  SITE (1)..(16): /note=“Variant residues given in the sequence have no preference with respect to those in the annotations for variant positions”
  source: /note=“See specification as filed for detailed description of substitutions and preferred embodiments”
XLTFXEYWAQXXSAAX

SEQ ID NO: 7 - protein, 23 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“ ”
  MOD_RES (13)..(13): S-pentenyl alanine or any amino acid
  MOD_RES (17)..(17): S-pentenyl alanine or any amino acid
  MOD_RES (18)..(18): Norleucine
  MOD_RES (23)..(23): Lys-degron
  VARIANT (23)..(23): /replace=“ ”
  SITE (1)..(23): /note=“Variant residues given in the sequence have no preference with respect to those in the annotations for variant positions”
  source: /note=“See specification as filed for detailed description of substitutions and preferred embodiments”
XDIIRNIARHLAXVGDXXDRSIX

SEQ ID NO: 8 - protein, 23 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“ ”
  MOD_RES (10)..(10): S-pentenyl alanine or any amino acid
  MOD_RES (14)..(14): S-pentenyl alanine or any amino acid
  MOD_RES (23)..(23): Lys-degron
  VARIANT (23)..(23): /replace=“ ”
  SITE (1)..(23): /note=“Variant residues given in the sequence have no preference with respect to those in the annotations for variant positions”
  source: /note=“See specification as filed for detailed description of substitutions and preferred embodiments”
XIWIAQELRXIGDXFNAYYARRX

SEQ ID NO: 9 - protein, 27 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“ ”
  MOD_RES (15)..(15): S-pentenyl alanine or any amino acid
  MOD_RES (16)..(16): Norleucine
  MOD_RES (19)..(19): S-pentenyl alanine or any amino acid
  MOD_RES (27)..(27): Lys-degron
  VARIANT (27)..(27): /replace=“ ”
  SITE (1)..(27): /note=“Variant residues given in the sequence have no preference with respect to those in the annotations for variant positions”
  source: /note=“See specification as filed for detailed description of substitutions and preferred embodiments”
XNLWAAQRYGRELRXXDDXFVDSFKKX

SEQ ID NO: 10 - protein, 27 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“Degron-Ahx” or “ ”
  MOD_RES (15)..(15): S-pentenyl alanine or any amino acid
  MOD_RES (16)..(16): Norleucine
  MOD_RES (19)..(19): S-pentenyl alanine or any amino acid
  MOD_RES (27)..(27): Lys-degron
  VARIANT (27)..(27): /replace=“Lys-ivDde” or “ ”
  SITE (1)..(27): /note=“Variant residues given in the sequence have no preference with respect to those in the annotations for variant positions”
  source: /note=“See specification as filed for detailed description of substitutions and preferred embodiments”
XNLWAAQRYGRELRXXSDXFVDSFKKX

SEQ ID NO: 11 - protein, 23 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“Degron-Ahx” or “ ”
  MOD_RES (10)..(10): S-pentenyl alanine or any amino acid
  MOD_RES (14)..(14): S-pentenyl alanine or any amino acid
  MOD_RES (20)..(20): Norleucine
  MOD_RES (23)..(23): Lys-degron
  VARIANT (23)..(23): /replace=“Lys-ivDde” or “ ”
  SITE (1)..(23): /note=“Variant residues given in the sequence have no preference with respect to those in the annotations for variant positions”
  source: /note=“See specification as filed for detailed description of substitutions and preferred embodiments”
XQLTAARLKXLGDXLHQRTXWRX

SEQ ID NO: 12 - protein, 27 aa, Artificial Sequence
  source: /note=“Description of Artificial Sequence: Synthetic peptide”
  source: /note=“May be N-term Acetylated”
  MOD_RES (1)..(1): Thalidomide-aminohexanoic
  VARIANT (1)..(1): /replace=“ ”
  MOD_RES (14)..(14): S-pentenyl alanine or any amino acid
  MOD_RES (18)..(18): S-pentenyl alanine or any amino acid
  MOD_RES (27)..(27): Lys-degron
  VARIANT (27)..(27): /replace=“ ”
  SITE (1)..(27): /note=“Variant residues given in the sequence have no preference with respect to those in the annotations for variant positions”
  source: /note=“See specification as filed for detailed description of substitutions and preferred embodiments”
XAELEVESATQLRXFGDXLNFRQKLLX

SEQ ID NO: 13 - protein, 26 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (13)..(13): S-pentenyl alanine or any amino acid
  MOD_RES (14)..(14): Norleucine
  MOD_RES (17)..(17): S-pentenyl alanine or any amino acid
  MOD_RES (26)..(26): Lys-epsilon-amino-thalidomide
  VARIANT (26)..(26): /replace=“Lys-ivDde” or “ ”
  SITE (1)..(26): /note=“Variant residues given in the sequence have no preference with respect to those in the annotations for variant positions”
  source: /note=“See specification as filed for detailed description of substitutions and preferred embodiments”
XQWAREIGAQLRXXADXLNAQYERRX

SEQ ID NO: 14 - protein, 29 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (7)..(7): S-pentenyl alanine or any amino acid
  MOD_RES (11)..(11): S-pentenyl alanine or any amino acid
  MOD_RES (29)..(29): Lys-epsilon-amino-thalidomide
  VARIANT (29)..(29): /replace=“Lys-ivDde” or “ ”
  SITE (1)..(29): /note=“Variant residues given in the sequence have no preference with respect to those in the annotations for variant positions”
  source: /note=“See specification as filed for detailed description of substitutions and preferred embodiments”
XFSSNRXKILXRTQILNQEWKQRRIQPVX

SEQ ID NO: 15 - protein, 20 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (8)..(8): S-pentenyl alanine or any amino acid
  MOD_RES (12)..(12): S-pentenyl alanine or any amino acid
  MOD_RES (20)..(20): Lys-epsilon-amino-thalidomide
  VARIANT (20)..(20): /replace=“Lys-ivDde” or “ ”
  SITE (1)..(20): /note=“Variant residues given in the sequence have no preference with respect to those in the annotations for variant positions”
  source: /note=“See specification as filed for detailed description of substitutions and preferred embodiments”
XRRFFGIXLTNXLKTEEGNX

SEQ ID NO: 16 - protein, 24 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (16)..(16): S-pentenyl alanine or any amino acid
  MOD_RES (20)..(20): S-pentenyl alanine or any amino acid
  MOD_RES (24)..(24): Lys-epsilon-amino-thalidomide
  VARIANT (24)..(24): /replace=“Lys-ivDde” or “ ”
  SITE (1)..(24): /note=“Variant residues given in the sequence have no preference with respect to those in the annotations for variant positions”
  source: /note=“See specification as filed for detailed description of substitutions and preferred embodiments”
XRKALETLRRVGDGVXRNHXTAFX

SEQ ID NO: 17 - protein, 26 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (15)..(15): S-pentenyl alanine or any amino acid
  MOD_RES (19)..(19): S-pentenyl alanine or any amino acid
  MOD_RES (23)..(23): Norleucine
  MOD_RES (26)..(26): Lys-epsilon-amino-thalidomide
  VARIANT (26)..(26): /replace=“Lys-ivDde” or “ ”
  SITE (1)..(26): /note=“Variant residues given in the sequence have no preference with respect to those in the annotations for variant positions”
  source: /note=“See specification as filed for detailed description of substitutions and preferred embodiments”
XLSQEQLEHRERSLXTLRXIQRXLFX

SEQ ID NO: 18 - protein, 16 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (5)..(5): R-octenyl alanine
  MOD_RES (11)..(11): Cyclobutylalanine
  MOD_RES (12)..(12): S-pentenyl alanine
  MOD_RES (16)..(16): Lys-epsilon-amino-thalidomide
  VARIANT (16)..(16): /replace=“Lys-ivDde” or “ ”
  SITE (1)..(16): /note=“Variant residues given in the sequence have no preference with respect to those in the annotations for variant positions”
  source: /note=“See specification as filed for detailed description of substitutions and preferred embodiments”
XLTFXEYWAQXXSAAX

SEQ ID NO: 19 - protein, 23 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (13)..(13): S-pentenyl alanine or any amino acid
  MOD_RES (17)..(17): S-pentenyl alanine or any amino acid
  MOD_RES (18)..(18): Norleucine
  MOD_RES (23)..(23): Lys-epsilon-amino-thalidomide
  VARIANT (23)..(23): /replace=“Lys-ivDde” or “ ”
  SITE (1)..(23): /note=“Variant residues given in the sequence have no preference with respect to those in the annotations for variant positions”
  source: /note=“See specification as filed for detailed description of substitutions and preferred embodiments”
XDIIRNIARHLAXVGDXXDRSIX

SEQ ID NO: 20 - protein, 23 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (10)..(10): S-pentenyl alanine or any amino acid
  MOD_RES (14)..(14): S-pentenyl alanine or any amino acid
  MOD_RES (23)..(23): Lys-epsilon-amino-thalidomide
  VARIANT (23)..(23): /replace=“Lys-ivDde” or “ ”
  SITE (1)..(23): /note=“Variant residues given in the sequence have no preference with respect to those in the annotations for variant positions”
  source: /note=“See specification as filed for detailed description of substitutions and preferred embodiments”
XIWIAQELRXIGDXFNAYYARRX

SEQ ID NO: 21 - protein, 27 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (15)..(15): S-pentenyl alanine or any amino acid
  MOD_RES (16)..(16): Norleucine
  MOD_RES (19)..(19): S-pentenyl alanine or any amino acid
  MOD_RES (27)..(27): Lys-epsilon-amino-thalidomide
  VARIANT (27)..(27): /replace=“Lys-ivDde” or “ ”
  SITE (1)..(27): /note=“Variant residues given in the sequence have no preference with respect to those in the annotations for variant positions”
  source: /note=“See specification as filed for detailed description of substitutions and preferred embodiments”
XNLWAAQRYGRELRXXDDXFVDSFKKX

SEQ ID NO: 22 - protein, 27 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (15)..(15): S-pentenyl alanine or any amino acid
  MOD_RES (16)..(16): Norleucine
  MOD_RES (19)..(19): S-pentenyl alanine or any amino acid
  MOD_RES (27)..(27): Lys-epsilon-amino-thalidomide
  VARIANT (27)..(27): /replace=“Lys-ivDde” or “ ”
  SITE (1)..(27): /note=“Variant residues given in the sequence have no preference with respect to those in the annotations for variant positions”
  source: /note=“See specification as filed for detailed description of substitutions and preferred embodiments”
XNLWAAQRYGRELRXXSDXFVDSFKKX

SEQ ID NO: 23 - protein, 23 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (10)..(10): S-pentenyl alanine or any amino acid
  MOD_RES (14)..(14): S-pentenyl alanine or any amino acid
  MOD_RES (20)..(20): Norleucine
  MOD_RES (23)..(23): Lys-epsilon-amino-thalidomide
  VARIANT (23)..(23): /replace=“Lys-ivDde” or “ ”
  SITE (1)..(23): /note=“Variant residues given in the sequence have no preference with respect to those in the annotations for variant positions”
  source: /note=“See specification as filed for detailed description of substitutions and preferred embodiments”
XQLTAARLKXLGDXLHQRTXWRX

SEQ ID NO: 24 - protein, 27 aa, Artificial Sequence
  source: /note=“Description of Artificial Sequence: Synthetic peptide”
  MOD_RES (1)..(1): Degron-Ahx
  VARIANT (1)..(1): /replace=“ ”
  MOD_RES (14)..(14): S-pentenyl alanine or any amino acid
  MOD_RES (18)..(18): S-pentenyl alanine or any amino acid
  MOD_RES (27)..(27): Lys-epsilon-amino-thalidomide
  VARIANT (27)..(27): /replace=“Lys-ivDde” or “ ”
  SITE (1)..(27): /note=“Variant residues given in the sequence have no preference with respect to those in the annotations for variant positions”
  source: /note=“See specification as filed for detailed description of substitutions and preferred embodiments”
XAELEVESATQLRXFGDXLNFRQKLLX

SEQ ID NO: 25 - protein, 7 aa, Artificial Sequence
  source: /note=“Description of Artificial Sequence: Synthetic peptide”
DQIVPEY

SEQ ID NO: 26 - protein, 6 aa, Artificial Sequence
  source: /note=“Description of Artificial Sequence: Synthetic peptide”
GFDVPD

SEQ ID NO: 27 - protein, 6 aa, Artificial Sequence
  source: /note=“Description of Artificial Sequence: Synthetic peptide”
GFDAAD

SEQ ID NO: 28 - protein, 6 aa, Artificial Sequence
  source: /note=“Description of Artificial Sequence: Synthetic peptide”
GNDVPD

SEQ ID NO: 29 - protein, 6 aa, Artificial Sequence
  source: /note=“Description of Artificial Sequence: Synthetic peptide”
PQTVPD

SEQ ID NO: 30 - protein, 6 aa, Artificial Sequence
  source: /note=“Description of Artificial Sequence: Synthetic peptide”
DQIVPD

SEQ ID NO: 31 - protein, 8 aa, Artificial Sequence
  source: /note=“Description of Artificial Sequence: Synthetic peptide”
FSDLWKLL

SEQ ID NO: 32 - protein, 8 aa, Artificial Sequence
  source: /note=“Description of Artificial Sequence: Synthetic peptide”
SVEQTPKK

SEQ ID NO: 33 - protein, 8 aa, Artificial Sequence
  source: /note=“Description of Artificial Sequence: Synthetic peptide”
LLPTPPLS

SEQ ID NO: 34 - protein, 5 aa, Artificial Sequence
  source: /note=“Description of Artificial Sequence: Synthetic peptide”
ASSSS

SEQ ID NO: 35 - protein, 14 aa, Artificial Sequence
  source: /note=“Description of Artificial Sequence: Synthetic peptide”
LAPAAGDTIISLDF

SEQ ID NO: 36 - protein, 8 aa, Artificial Sequence
  source: /note=“Description of Artificial Sequence: Synthetic peptide”
PFLTPSPE

SEQ ID NO: 37 - protein, 4 aa, Artificial Sequence
  source: /note=“Description of Artificial Sequence: Synthetic peptide”
PPPY

SEQ ID NO: 38 - protein, 6 aa, Artificial Sequence
  source: /note=“Description of Artificial Sequence: Synthetic peptide”
DEETGE

SEQ ID NO: 39 - protein, 7 aa, Artificial Sequence
  source: /note=“Description of Artificial Sequence: Synthetic peptide”
QDIDLGV

SEQ ID NO: 40 - protein, 10 aa, Artificial Sequence
  source: /note=“Description of Artificial Sequence: Synthetic peptide”
LLQPNNYQFC

SEQ ID NO: 41 - protein, 10 aa, Artificial Sequence
  source: /note=“Description of Artificial Sequence: Synthetic peptide”
RAVENQYSFY

SEQ ID NO: 42 - protein, 5 aa, Artificial Sequence
  source: /note=“Description of Artificial Sequence: Synthetic peptide”
QKENS

SEQ ID NO: 43 - protein, 6 aa, Artificial Sequence
  source: /note=“Description of Artificial Sequence: Synthetic peptide”
FDIYMD

SEQ ID NO: 44 - protein, 9 aa, Artificial Sequence
  source: /note=“Description of Artificial Sequence: Synthetic peptide”
PRTALGDIG

SEQ ID NO: 45 - protein, 5 aa, Artificial Sequence
  source: /note=“Description of Artificial Sequence: Synthetic peptide”
DKENG

SEQ ID NO: 46 - protein, 5 aa, Artificial Sequence
  source: /note=“Description of Artificial Sequence: Synthetic peptide”
  MOD_RES (1)..(1): Phospho-Ser
  VARIANT (1)..(1): /replace=“phospho-Thr”
  MOD_RES (3)..(3): Any amino acid
  MOD_RES (4)..(4): Any amino acid
  VARIANT (5)..(5): /replace=“phospho-Ser” or “phospho-Thr”
  SITE (1)..(5): /note=“Variant residues given in the sequence have no preference with respect to those in the annotations for variant positions”
SPXXE

SEQ ID NO: 47 - protein, 4 aa, Artificial Sequence
  source: /note=“Description of Artificial Sequence: Synthetic peptide”
GGGS

SEQ ID NO: 48 - protein, 4 aa, Artificial Sequence
  source: /note=“Description of Artificial Sequence: Synthetic peptide”
SGGG

SEQ ID NO: 49 - protein, 5 aa, Artificial Sequence
  source: /note=“Description of Artificial Sequence: Synthetic peptide”
GGGGS

SEQ ID NO: 50 - protein, 5 aa, Artificial Sequence
  source: /note=“Description of Artificial Sequence: Synthetic peptide”
SGGGG

SEQ ID NO: 51 - protein, 6 aa, Artificial Sequence
  source: /note=“Description of Artificial Sequence: Synthetic peptide”
GGGGGS

SEQ ID NO: 52 - protein, 6 aa, Artificial Sequence
  source: /note=“Description of Artificial Sequence: Synthetic peptide”
SGGGGG

SEQ ID NO: 53 - protein, 7 aa, Artificial Sequence
  source: /note=“Description of Artificial Sequence: Synthetic peptide”
GGGGGGS

SEQ ID NO: 54 - protein, 7 aa, Artificial Sequence
  source: /note=“Description of Artificial Sequence: Synthetic peptide”
SGGGGGG

SEQ ID NO: 55 - protein, 25 aa, Artificial Sequence
  source: /note=“Description of Artificial Sequence: Synthetic peptide”
  MOD_RES (4)..(5): Any amino acid
  MOD_RES (9)..(10): Any amino acid
  MOD_RES (14)..(15): Any amino acid
  MOD_RES (19)..(20): Any amino acid
  SITE (1)..(20): /note=“This region may encompass 0-4 ′Gly Gly Gly Xaa Xaa′ repeating units”
  source: /note=“See specification as filed for detailed description of substitutions and preferred embodiments”
GGGXXGGGXXGGGXXGGGXXGGGGS

SEQ ID NO: 56 - protein, 25 aa, Artificial Sequence
  source: /note=“Description of Artificial Sequence: Synthetic peptide”
  MOD_RES (6)..(6): Any amino acid
  MOD_RES (11)..(11): Any amino acid
  MOD_RES (16)..(16): Any amino acid
  MOD_RES (21)..(21): Any amino acid
  SITE (6)..(25): /note=“This region may encompass 0-4 ′Xaa Gly Gly Gly Ser′ repeating units”
  source: /note=“See specification as filed for detailed description of substitutions and preferred embodiments”
GGGGSXGGGSXGGGSXGGGSXGGGS

SEQ ID NO: 57 - protein, 25 aa, Artificial Sequence
  source: /note=“Description of Artificial Sequence: Synthetic peptide”
  SITE (1)..(20): /note=“This region may encompass 0-4 ′Gly Gly Gly Pro Ser′ repeating units”
GGGPSGGGPSGGGPSGGGPSGGGGS

SEQ ID NO: 58 - protein, 25 aa, Artificial Sequence
  source: /note=“Description of Artificial Sequence: Synthetic peptide”
  SITE (1)..(20): /note=“This region may encompass 0-4 ′Gly Gly Gly Gly Gln′ repeating units”
GGGGQGGGGQGGGGQGGGGQGGGGS

SEQ ID NO: 59 - protein, 25 aa, Artificial Sequence
  source: /note=“Description of Artificial Sequence: Synthetic peptide”
  SITE (1)..(20): /note=“This region may encompass 0-4 ′Gly Gly Gly Gly Ala′ repeating units”
GGGGAGGGGAGGGGAGGGGAGGGGS

SEQ ID NO: 60 - protein, 25 aa, Artificial Sequence
  source: /note=“Description of Artificial Sequence: Synthetic peptide”
  SITE (6)..(25): /note=“This region may encompass 0-4 ′Pro Gly Gly Gly Ser′ repeating units”
GGGGSPGGGSPGGGSPGGGSPGGGS

SEQ ID NO: 61 - protein, 15 aa, Artificial Sequence
  source: /note=“Description of Artificial Sequence: Synthetic peptide”
GGGGAGGGGAGGGGS

SEQ ID NO: 62 - protein, 15 aa, Artificial Sequence
  source: /note=“Description of Artificial Sequence: Synthetic peptide”
GGGGQGGGGQGGGGS

SEQ ID NO: 63 - protein, 15 aa, Artificial Sequence
  source: /note=“Description of Artificial Sequence: Synthetic peptide”
GGGPSGGGPSGGGGS

SEQ ID NO: 64 - protein, 15 aa, Artificial Sequence
  source: /note=“Description of Artificial Sequence: Synthetic peptide”
GGGGSPGGGSPGGGS

SEQ ID NO: 65 - protein, 9 aa, Artificial Sequence
  source: /note=“Description of Artificial Sequence: Synthetic peptide”
  MOD_RES (1)..(1): Any amino acid
  MOD_RES (3)..(3): Any amino acid
  MOD_RES (4)..(4): Any amino acid
  MOD_RES (6)..(6): Any amino acid
  MOD_RES (7)..(7): Any amino acid
  VARIANT (8)..(8): /replace=“Ile” or “Val” or “Met”
  MOD_RES (9)..(9): Any amino acid
  SITE (1)..(9): /note=“Variant residues given in the sequence have no preference with respect to those in the annotations for variant positions”
XRXXLXXLX

SEQ ID NO: 66 - protein, 5 aa, Artificial Sequence
  source: /note=“Description of Artificial Sequence: Synthetic peptide”
  MOD_RES (1)..(1): Any amino acid
  MOD_RES (5)..(5): Any amino acid
XKENX

SEQ ID NO: 67 - protein, 10 aa, Artificial Sequence
  source: /note=“Description of Artificial Sequence: Synthetic peptide”
  VARIANT (1)..(1): /replace=“Ile” or “Val” or “Leu”
  MOD_RES (2)..(2): Any amino acid
  VARIANT (3)..(3): /replace=“Leu” or “Met” or “Val” or “Pro”
  VARIANT (4)..(4): /replace=“His” or “Tyr”
  MOD_RES (5)..(5): Any amino acid
  VARIANT (6)..(6): /replace=“Glu”
  MOD_RES (7)..(7): Any amino acid
  MOD_RES (8)..(8): Any amino acid
  MOD_RES (9)..(9): Any amino acid
  SITE (7)..(9): /note=“This region may encompass 0-3 residues”
  VARIANT (10)..(10): /replace=“Glu” or “Ser” or “Thr”
  SITE (1)..(10): /note=“Variant residues given in the sequence have no preference with respect to those in the annotations for variant positions”
FXIFXDXXXD

SEQ ID NO: 68 - protein, 3 aa, Artificial Sequence
  source: /note=“Description of Artificial Sequence: Synthetic peptide”
  MOD_RES (1)..(1): Any amino acid
  VARIANT (2)..(2): /replace=“Leu” or “Met”
  SITE (1)..(3): /note=“Variant residues given in the sequence have no preference with respect to those in the annotations for variant positions”
XIR

SEQ ID NO: 69 - protein, 7 aa, Artificial Sequence
  source: /note=“Description of Artificial Sequence: Synthetic peptide”
  VARIANT (1)..(1): /replace=“Asn”
  MOD_RES (2)..(2): Any amino acid
  VARIANT (4)..(4): /replace=“Thr”
  MOD_RES (5)..(5): Any amino acid
  MOD_RES (6)..(6): Any amino acid
  SITE (1)..(7): /note=“Variant residues given in the sequence have no preference with respect to those in the annotations for variant positions”
DXYSXXP

SEQ ID NO: 70 - protein, 3 aa, Artificial Sequence
  source: /note=“Description of Artificial Sequence: Synthetic peptide”
DYR

SEQ ID NO: 71 - protein, 8 aa, Artificial Sequence
  source: /note=“Description of Artificial Sequence: Synthetic peptide”
  VARIANT (1)..(1): /replace=“Glu”
  VARIANT (2)..(2): /replace=“Glu”
  MOD_RES (3)..(3): Any amino acid
  MOD_RES (4)..(4): Any amino acid
  MOD_RES (5)..(5): Any amino acid
  SITE (3)..(5): /note=“This region may encompass 2-3 residues”
  VARIANT (8)..(8): /replace=“Glu”
  SITE (1)..(8): /note=“Variant residues given in the sequence have no preference with respect to those in the annotations for variant positions”
DDXXXVPD

SEQ ID NO: 72 - protein, 15 aa, Artificial Sequence
  source: /note=“Description of Artificial Sequence: Synthetic peptide”
  VARIANT (1)..(1): /replace=“Gln” or “ ”
  MOD_RES (2)..(2): Any amino acid
  MOD_RES (3)..(3): Any amino acid
  VARIANT (4)..(4): /replace=“Leu” or “Met” or “Val”
  VARIANT (5)..(5): /replace=“Thr”
  VARIANT (6)..(6): /replace=“Glu” or “Asn”
  VARIANT (7)..(7): /replace=“Tyr”
  VARIANT (8)..(8): /replace=“Tyr”
  MOD_RES (9)..(9): Any amino acid
  MOD_RES (10)..(10): Any amino acid
  MOD_RES (11)..(11): Any amino acid
  SITE (9)..(11): /note=“This region may encompass 2-3 residues”
  VARIANT (12)..(12): /replace=“Arg”
  VARIANT (13)..(13): /replace=“Arg”
  VARIANT (14)..(14): /replace=“Arg”
  SITE (12)..(14): /note=“This region may encompass 2-3 residues”
  MOD_RES (15)..(15): Any amino acid except Asp or Glu
  SITE (1)..(15): /note=“Variant residues given in the sequence have no preference with respect to those in the annotations for variant positions”
NXXISDFFXXXKKKX

SEQ ID NO: 73 - protein, 15 aa, Artificial Sequence
  source: /note=“Description of Artificial Sequence: Synthetic peptide”
  VARIANT (1)..(1): /replace=“Gln” or “ ”
  MOD_RES (2)..(2): Any amino acid
  MOD_RES (3)..(3): Any amino acid
  VARIANT (4)..(4): /replace=“Leu” or “Met” or “Val”
  VARIANT (6)..(6): /replace=“Glu” or “Asn”
  VARIANT (7)..(7): /replace=“Met” or “Phe” or “Tyr”
  VARIANT (8)..(8): /replace=“Met” or “Tyr”
  MOD_RES (9)..(9): Any amino acid
  MOD_RES (10)..(10): Any amino acid
  MOD_RES (11)..(11): Any amino acid
  SITE (9)..(11): /note=“This region may encompass 2-3 residues”
  VARIANT (12)..(12): /replace=“Arg”
  VARIANT (13)..(13): /replace=“Arg”
  VARIANT (14)..(14): /replace=“Arg”
  SITE (12)..(14): /note=“This region may encompass 2-3 residues”
  MOD_RES (15)..(15): Any amino acid except Asp or Glu
  SITE (1)..(15): /note=“Variant residues given in the sequence have no preference with respect to those in the annotations for variant positions”
NXXITDHFXXXKKKX

SEQ ID NO: 74 - protein, 6 aa, Artificial Sequence
  source: /note=“Description of Artificial Sequence: Synthetic peptide”
  VARIANT (1)..(1): /replace=“Asn” or “Ser”
  MOD_RES (2)..(2): Any amino acid
  VARIANT (3)..(3): /replace=“Glu” or “Ser”
  VARIANT (4)..(4): /replace=“Asn” or “Ser”
  SITE (1)..(6): /note=“Variant residues given in the sequence have no preference with respect to those in the annotations for variant positions”
DXDTGE

SEQ ID NO: 75 - protein, 7 aa, Artificial Sequence
  source: /note=“Description of Artificial Sequence: Synthetic peptide”
  MOD_RES (3)..(3): Any amino acid
QDXDLGV

SEQ ID NO: 76 - protein, 5 aa, Artificial Sequence
  source: /note=“Description of Artificial Sequence: Synthetic peptide”
  VARIANT (1)..(1): /replace=“Pro”
  VARIANT (3)..(3): /replace=“Val”
  VARIANT (4)..(4): /replace=“Met”
  SITE (1)..(5): /note=“Variant residues given in the sequence have no preference with respect to those in the annotations for variant positions”
APMIV

SEQ ID NO: 77 - protein, 10 aa, Artificial Sequence
  source: /note=“Description of Artificial Sequence: Synthetic peptide”
  MOD_RES (2)..(2): Any amino acid
  MOD_RES (5)..(5): Any amino acid
  VARIANT (7)..(7): /replace=“Val”
  SITE (1)..(10): /note=“Variant residues given in the sequence have no preference with respect to those in the annotations for variant positions”
EXEEXEADQH

SEQ ID NO: 78 - protein, 9 aa, Artificial Sequence
  source: /note=“Description of Artificial Sequence: Synthetic peptide”
  MOD_RES (2)..(2): Any amino acid except Pro
  MOD_RES (3)..(3): Any amino acid except Pro
  MOD_RES (4)..(4): Any amino acid except Pro
  MOD_RES (6)..(6): Any amino acid except Pro
  MOD_RES (7)..(7): Any amino acid except Pro
  MOD_RES (8)..(8): Any amino acid except Pro
  SITE (6)..(8): /note=“This region may encompass 2-3 residues”
  VARIANT (9)..(9): /replace=“Ile” or “Leu”
  SITE (1)..(9): /note=“Variant residues given in the sequence have no preference with respect to those in the annotations for variant positions”
FXXXWXXXV

SEQ ID NO: 79 - protein, 3 aa, Artificial Sequence
  source: /note=“Description of Artificial Sequence: Synthetic peptide”
  VARIANT (1)..(1): /replace=“ ”
  VARIANT (2)..(2): /replace=“Tyr” or “Leu” or “Ile” or “Trp”
  MOD_RES (3)..(3): Any amino acid except Pro
  SITE (1)..(3): /note=“Variant residues given in the sequence have no preference with respect to those in the annotations for variant positions”
MFX

SEQ ID NO: 80 - protein, 4 aa, Artificial Sequence
  source: /note=“Description of Artificial Sequence: Synthetic peptide”
  VARIANT (1)..(1): /replace=“ ”
  VARIANT (2)..(2): /replace=“Lys”
  MOD_RES (3)..(3): Any amino acid except Pro
  MOD_RES (4)..(4): Any amino acid
  SITE (1)..(4): /note=“Variant residues given in the sequence have no preference with respect to those in the annotations for variant positions”
MRXX

SEQ ID NO: 81 - protein, 3 aa, Artificial Sequence
  source: /note=“Description of Artificial Sequence: Synthetic peptide”
  VARIANT (1)..(1): /replace=“ ”
  VARIANT (2)..(2): /replace=“Asp”
  MOD_RES (3)..(3): Any amino acid
  SITE (1)..(3): /note=“Variant residues given in the sequence have no preference with respect to those in the annotations for variant positions”
MEX

SEQ ID NO: 82 - protein, 3 aa, Artificial Sequence
  source: /note=“Description of Artificial Sequence: Synthetic peptide”
  VARIANT (1)..(1): /replace=“ ”
  VARIANT (2)..(2): /replace=“Gln”
  MOD_RES (3)..(3): Any amino acid
  SITE (1)..(3): /note=“Variant residues given in the sequence have no preference with respect to those in the annotations for variant positions”
MNX

SEQ ID NO: 83 - protein, 3 aa, Artificial Sequence
  source: /note=“Description of Artificial Sequence: Synthetic peptide”
  VARIANT (1)..(1): /replace=“ ”
  MOD_RES (3)..(3): Any amino acid
  SITE (1)..(3): /note=“Variant residues given in the sequence have no preference with respect to those in the annotations for variant positions”
MCX

SEQ ID NO: 84 - protein, 14 aa, Artificial Sequence
  source: /note=“Description of Artificial Sequence: Synthetic peptide”
  VARIANT (1)..(1): /replace=“Leu”
  MOD_RES (4)..(4): Any amino acid
  MOD_RES (5)..(5): Any amino acid
  MOD_RES (6)..(6): Any amino acid
  MOD_RES (7)..(7): Any amino acid
  MOD_RES (8)..(8): Any amino acid
  MOD_RES (9)..(9): Any amino acid
  MOD_RES (10)..(10): Any amino acid
  MOD_RES (11)..(11): Any amino acid
  SITE (4)..(11): /note=“This region may encompass 6-8 residues”
  VARIANT (12)..(12): /replace=“Leu” or “Ile” or “Val” or “Met”
  MOD_RES (13)..(13): Any amino acid
  VARIANT (14)..(14): /replace=“Leu” or “Ile” or “Val” or “Met”
  SITE (1)..(14): /note=“Variant residues given in the sequence have no preference with respect to those in the annotations for variant positions”
IAPXXXXXXXXFXF

SEQ ID NO: 85 - protein, 18 aa, Artificial Sequence
  source: /note=“Description of Artificial Sequence: Synthetic peptide”
  MOD_RES (1)..(1): Any amino acid
  MOD_RES (2)..(2): Any amino acid
  VARIANT (3)..(3): /replace=“Lys”
  VARIANT (4)..(4): /replace=“Lys”
  MOD_RES (5)..(5): Any amino acid
  MOD_RES (8)..(8): Any amino acid
  MOD_RES (9)..(9): Any amino acid
  VARIANT (11)..(11): /replace=“Leu” or “Met”
  MOD_RES (12)..(12): Any amino acid
  VARIANT (13)..(13): /replace=“Lys”
  VARIANT (15)..(15): /replace=“Arg” or “Lys”
  MOD_RES (16)..(16): Any amino acid
  VARIANT (17)..(17): /replace=“Lys”
  MOD_RES (18)..(18): Any amino acid
  SITE (1)..(18): /note=“Variant residues given in the sequence have no preference with respect to those in the annotations for variant positions”
XXRRXSLXXFFXRRHXRX

SEQ ID NO: 86 - protein, 8 aa, Artificial Sequence
  source: /note=“Description of Artificial Sequence: Synthetic peptide”
  VARIANT (1)..(1): /replace=“Ile” or “Val” or “Met” or “Pro”
  MOD_RES (2)..(2): Any amino acid
  MOD_RES (3)..(3): Any amino acid
  SITE (2)..(3): /note=“This region may encompass 0-2 residues”
  MOD_RES (6)..(6): Any amino acid
  MOD_RES (7)..(7): Any amino acid
  VARIANT (8)..(8): /replace=“Thr”
  SITE (1)..(8): /note=“Variant residues given in the sequence have no preference with respect to those in the annotations for variant positions”
LXXTPXXS

SEQ ID NO: 87 - protein, 8 aa, Artificial Sequence
  source: /note=“Description of Artificial Sequence: Synthetic peptide”
  VARIANT (1)..(1): /replace=“Ile” or “Val” or “Met” or “Pro”
  MOD_RES (2)..(2): Any amino acid
  MOD_RES (3)..(3): Any amino acid
  SITE (2)..(3): /note=“This region may encompass 0-2 residues”
  MOD_RES (6)..(6): Any amino acid
  MOD_RES (7)..(7): Any amino acid
  SITE (1)..(8): /note=“Variant residues given in the sequence have no preference with respect to those in the annotations for variant positions”
LXXTPXXE

SEQ ID NO: 88 - protein, 8 aa, Artificial Sequence
  source: /note=“Description of Artificial Sequence: Synthetic peptide”
  MOD_RES (1)..(1): Any amino acid
  MOD_RES (2)..(2): Any amino acid
  VARIANT (3)..(3): /replace=“Glu”
  MOD_RES (4)..(4): Any amino acid
  MOD_RES (7)..(7): Any amino acid
  SITE (1)..(8): /note=“Variant residues given in the sequence have no preference with respect to those in the annotations for variant positions”
XXDXTPXK

SEQ ID NO: 89 - protein, 13 aa, Artificial Sequence
  source: /note=“Description of Artificial Sequence: Synthetic peptide”
  MOD_RES (1)..(1): Any amino acid
  VARIANT (2)..(2): /replace=“Leu” or “Ile” or “Ala”
  VARIANT (3)..(3): /replace=“Leu” or “Ile”
  VARIANT (8)..(8): /replace=“Leu” or “Ile”
  MOD_RES (9)..(9): Any amino acid
  MOD_RES (10)..(10): Any amino acid
  MOD_RES (11)..(11): Any amino acid
  MOD_RES (13)..(13): Any amino acid
  SITE (1)..(13): /note=“Variant residues given in the sequence have no preference with respect to those in the annotations for variant positions”
XVVGWPPVXXXRX

SEQ ID NO: 90 - protein, 7 aa, Artificial Sequence
  source: /note=“Description of Artificial Sequence: Synthetic peptide”
  MOD_RES (4)..(4): Any amino acid
  MOD_RES (5)..(5): Any amino acid
  MOD_RES (6)..(6): Any amino acid
  SITE (4)..(6): /note=“This region may encompass 2-3 residues”
  VARIANT (7)..(7): /replace=“Thr”
  SITE (1)..(7): /note=“Variant residues given in the sequence have no preference with respect to those in the annotations for variant positions”
DSGXXXS

SEQ ID NO: 91 - protein, 9 aa, Artificial Sequence
  source: /note=“Description of Artificial Sequence: Synthetic peptide”
  MOD_RES (1)..(1): Any amino acid
  MOD_RES (3)..(3): Any amino acid
  MOD_RES (5)..(5): Any amino acid
  MOD_RES (7)..(7): Any amino acid
  MOD_RES (9)..(9): Any amino acid except Pro
XPXAXVXPX

SEQ ID NO: 92 - protein, 5 aa, Artificial Sequence
  source: /note=“Description of Artificial Sequence: Synthetic peptide”
  VARIANT (1)..(1): /replace=“Val” or “Pro”
  MOD_RES (2)..(2): Any amino acid
  VARIANT (3)..(3): /replace=“Thr”
  VARIANT (4)..(4): /replace=“Thr”
  VARIANT (5)..(5): /replace=“Thr”
  SITE (1)..(5): /note=“Variant residues given in the sequence have no preference with respect to those in the annotations for variant positions”
AXSSS

SEQ ID NO: 93 - protein, 9 aa, Artificial Sequence
  source: /note=“Description of Artificial Sequence: Synthetic peptide”
HRKHLQEIP

SEQ ID NO: 94 - protein, 5 aa, Artificial Sequence
  source: /note=“Description of Artificial Sequence: Synthetic peptide”
SKENV

SEQ ID NO: 95 - protein, 4 aa, Artificial Sequence
  source: /note=“Description of Artificial Sequence: Synthetic peptide”
TRIR

SEQ ID NO: 96 - protein, 7 aa, Artificial Sequence
  source: /note=“Description of Artificial Sequence: Synthetic peptide”
DQIVPEY

SEQ ID NO: 97 - protein, 13 aa, Artificial Sequence
  source: /note=“Description of Artificial Sequence: Synthetic peptide”
TSMTDFYHSKRRL

SEQ ID NO: 98 - protein, 6 aa, Artificial Sequence
  source: /note=“Description of Artificial Sequence: Synthetic peptide”
SPETGE

SEQ ID NO: 99 - protein, 10 aa, Artificial Sequence
  source: /note=“Description of Artificial Sequence: Synthetic peptide”
EPEEPEADQH

SEQ ID NO: 100 - protein, 13 aa, Artificial Sequence
  source: /note=“Description of Artificial Sequence: Synthetic peptide”
LAPYIPMDDDFQL

SEQ ID NO: 101 - protein, 6 aa, Artificial Sequence
  source: /note=“Description of Artificial Sequence: Synthetic peptide”
LTPPQS

SEQ ID NO: 102 - protein, 8 aa, Artificial Sequence
  source: /note=“Description of Artificial Sequence: Synthetic peptide”
SVEQTPRK

SEQ ID NO: 103 - protein, 6 aa, Artificial Sequence
  source: /note=“Description of Artificial Sequence: Synthetic peptide”
DSGNYS

SEQ ID NO: 104 - protein, 9 aa, Artificial Sequence
  source: /note=“Description of Artificial Sequence: Synthetic peptide”
KPAAVVAPI

SEQ ID NO: 105 - protein, 5 aa, Artificial Sequence
  source: /note=“Description of Artificial Sequence: Synthetic peptide”
ADSST

SEQ ID NO: 106 - protein, 6 aa, Artificial Sequence
  source: /note=“Description of Artificial Sequence: Synthetic peptide”
LTPPAS

SEQ ID NO: 107 - protein, 6 aa, Artificial Sequence
  source: /note=“Description of Artificial Sequence: Synthetic peptide”
LTPPSS

SEQ ID NO: 108 - protein, 6 aa, Artificial Sequence
  source: /note=“Description of Artificial Sequence: Synthetic peptide”
LSPPPS

SEQ ID NO: 109 - protein, 6 aa, Artificial Sequence
  source: /note=“Description of Artificial Sequence: Synthetic peptide”
LSPPAS

SEQ ID NO: 110 - protein, 6 aa, Artificial Sequence
  source: /note=“Description of Artificial Sequence: Synthetic peptide”
LSPPLS

SEQ ID NO: 111 - protein, 6 aa, Artificial Sequence
  source: /note=“Description of Artificial Sequence: Synthetic peptide”
DSGIIS

SEQ ID NO: 112 - protein, 6 aa, Artificial Sequence
  source: /note=“Description of Artificial Sequence: Synthetic peptide”
DSGNYT

SEQ ID NO: 113 - protein, 6 aa, Artificial Sequence
  source: /note=“Description of Artificial Sequence: Synthetic peptide”
DSGIDT

SEQ ID NO: 114 - protein, 6 aa, Artificial Sequence
  source: /note=“Description of Artificial Sequence: Synthetic peptide”
DSGIET

SEQ ID NO: 115 - protein, 7 aa, Artificial Sequence
  source: /note=“Description of Artificial Sequence: Synthetic peptide”
DSGVDTS

SEQ ID NO: 116 - protein, 13 aa, Artificial Sequence
  source: /note=“Description of Artificial Sequence: Synthetic peptide”
TSMTDFYHSKRRI

SEQ ID NO: 117 - protein, 13 aa, Artificial Sequence
  source: /note=“Description of Artificial Sequence: Synthetic peptide”
TSMTDFYHSKRKL

SEQ ID NO: 118 - protein, 13 aa, Artificial Sequence
  source: /note=“Description of Artificial Sequence: Synthetic peptide”
TSMTDFYHSKRRS

SEQ ID NO: 119 - protein, 23 aa, Artificial Sequence
  source: /note=“Description of Artificial Sequence: Synthetic peptide”
  MOD_RES (4)..(4): R-octenyl alanine
  MOD_RES (10)..(10): Cyclobutylalanine
  MOD_RES (11)..(11): S-pentenyl alanine
  MOD_RES (15)..(15): Aminohexanoic acid
  MOD_RES (16)..(16): Aminohexanoic acid
LTFXEYWAQXXSAAXXDQIVPEY

SEQ ID NO: 120 - protein, 22 aa, Artificial Sequence
  source: /note=“Description of Artificial Sequence: Synthetic peptide”
  MOD_RES (4)..(4): R-octenyl alanine
  MOD_RES (10)..(10): Cyclobutylalanine
  MOD_RES (11)..(11): S-pentenyl alanine
  MOD_RES (15)..(15): Aminohexanoic acid
LTFXEYWAQXXSAAXDQIVPEY

SEQ ID NO: 121 - protein, 23 aa, Artificial Sequence
  source: /note=“Description of Artificial Sequence: Synthetic peptide”
  MOD_RES (8)..(8): Aminohexanoic acid
  MOD_RES (9)..(9): Aminohexanoic acid
  MOD_RES (13)..(13): R-octenyl alanine
  MOD_RES (19)..(19): Cyclobutylalanine
  MOD_RES (20)..(20): S-pentenyl alanine
DQIVPEYXXLTFXEYWAQXXSAA

SEQ ID NO: 122 - protein, 22 aa, Artificial Sequence
  source: /note=“Description of Artificial Sequence: Synthetic peptide”
  MOD_RES (8)..(8): Aminohexanoic acid
  MOD_RES (12)..(12): R-octenyl alanine
  MOD_RES (18)..(18): Cyclobutylalanine
  MOD_RES (19)..(19): S-pentenyl alanine
DQIVPEYXLTFXEYWAQXXSAA

SEQ ID NO: 123 - protein, 30 aa, Artificial Sequence
  source: /note=“Description of Artificial Sequence: Synthetic polypeptide”
  MOD_RES (9)..(9): S-pentenyl alanine
  MOD_RES (13)..(13): S-pentenyl alanine
  MOD_RES (22)..(22): Aminohexanoic acid
  MOD_RES (23)..(23): Aminohexanoic acid
IWIAQELRXIGDXFNAYYARRXXDQIVPEY

SEQ ID NO: 124 - protein, 29 aa, Artificial Sequence
  source: /note=“Description of Artificial Sequence: Synthetic peptide”
  MOD_RES (9)..(9): S-pentenyl alanine
  MOD_RES (13)..(13): S-pentenyl alanine
  MOD_RES (22)..(22): Aminohexanoic acid
IWIAQELRXIGDXFNAYYARRXDQIVPEY

SEQ ID NO: 125 - protein, 30 aa, Artificial Sequence
  source: /note=“Description of Artificial Sequence: Synthetic polypeptide”
  MOD_RES (8)..(8): Aminohexanoic acid
  MOD_RES (9)..(9): Aminohexanoic acid
  MOD_RES (18)..(18): S-pentenyl alanine
  MOD_RES (22)..(22): S-pentenyl alanine
DQIVPEYXXIWIAQELRXIGDXFNAYYARR

SEQ ID NO: 126 - protein, 29 aa, Artificial Sequence
  source: /note=“Description of Artificial Sequence: Synthetic peptide”
  MOD_RES (8)..(8): Aminohexanoic acid
  MOD_RES (17)..(17): S-pentenyl alanine
  MOD_RES (21)..(21): S-pentenyl alanine
DQIVPEYXIWIAQELRXIGDXFNAYYARR

SEQ ID NO: 127 - protein, 21 aa, Artificial Sequence
  source: /note=“Description of Artificial Sequence: Synthetic peptide”
  MOD_RES (5)..(5): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal
  MOD_RES (9)..(9): (S)-2-(4-pentenyl)alanine
  MOD_RES (13)..(13): (S)-2-(4-pentenyl)alanine
IWIAXELRXIGDXFNAYYARR

SEQ ID NO: 128 - protein, 21 aa, Artificial Sequence
  source: /note=“Description of Artificial Sequence: Synthetic peptide”
  MOD_RES (9)..(9): (S)-2-(4-pentenyl)alanine
  MOD_RES (13)..(13): (S)-2-(4-pentenyl)alanine
  MOD_RES (16)..(16): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal
IWIAQELRXIGDXFNXYYARR

SEQ ID NO: 129 - protein, 14 aa, Artificial Sequence
  source: /note=“Description of Artificial Sequence: Synthetic peptide”
  MOD_RES (4)..(4): (R)-2-(7-octenyl)alanine
  MOD_RES (5)..(5): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal; continued below;
  MOD_RES (5)..(5): Continued from above; DAB-TRIB carboxylate, DAB-Gly-TRIB carboxylate, DAB-beta-Ala-TRIB carboxylate, DAB-Linker3- TRIB carboxylate, DAB-Linker5-TRIB carboxylate, DAB-Linker7- TRIB carboxylate
  MOD_RES (11)..(11): (S)-2-(4-pentenyl)alanine
LTFXXYWAQLXSAA

SEQ ID NO: 130 - protein, 14 aa, Artificial Sequence
  source: /note=“Description of Artificial Sequence: Synthetic peptide”
  MOD_RES (4)..(4): (R)-2-(7-octenyl)alanine
  MOD_RES (11)..(11): (S)-2-(4-pentenyl)alanine
  MOD_RES (12)..(12): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal; continued below;
  MOD_RES (12)..(12): Continued from above; DAB-VHL carboxylate, DAB-Gly-VHL carboxylate, DAB-beta-Ala-VHL carboxylate, DAB-Linker3-VHL carboxylate, DAB-Linker5-VHL carboxylate, and DAB-Linker7-VHL carboxylate
LTFXEYWAQLXXAA

SEQ ID NO: 131 - protein, 14 aa, Artificial Sequence
  source: /note=“Description of Artificial Sequence: Synthetic peptide”
  MOD_RES (1)..(1): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal; continued below;
  MOD_RES (1)..(1): Continued from above; DAB-VHL carboxylate, DAB-Gly-VHL carboxylate, DAB-beta-Ala-VHL carboxylate, DAB-Linker3-VHL carboxylate, DAB-Linker5-VHL carboxylate, and DAB-Linker7-VHL carboxylate
  MOD_RES (4)..(4): (R)-2-(7-octenyl)alanine
  MOD_RES (11)..(11): (S)-2-(4-pentenyl)alanine
XTFXEYWAQLXSAA

SEQ ID NO: 132 - protein, 14 aa, Artificial Sequence
  source: /note=“Description of Artificial Sequence: Synthetic peptide”
  MOD_RES (4)..(4): R-octenyl alanine
  MOD_RES (10)..(10): Cyclobutylalanine
  MOD_RES (11)..(11): S-pentenyl alanine
LTFXEYWAQXXSAA

SEQ ID NO: 133 - protein, 21 aa, Artificial Sequence
  source: /note=“Description of Artificial Sequence: Synthetic peptide”
  MOD_RES (9)..(9): (S)-2-(4-pentenyl)alanine
  MOD_RES (13)..(13): (S)-2-(4-pentenyl)alanine
  MOD_RES (19)..(19): Norleucine
QLTAARLKXLGDXLHQRTXWR

SEQ ID NO: 134 - protein, 14 aa, Artificial Sequence
  source: /note=“Description of Artificial Sequence: Synthetic peptide”
  MOD_RES (4)..(4): R-octenyl alanine
  MOD_RES (11)..(11): S-pentenyl alanine
LTFXEYWAQLXSAA

SEQ ID NO: 135 - skipped

SEQ ID NO: 136 - protein, 21 aa, Artificial Sequence
  source: /note=“Description of Artificial Sequence: Synthetic peptide”
  MOD_RES (5)..(5): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal
  MOD_RES (9)..(9): Any amino acid
  MOD_RES (13)..(13): Any amino acid
IWIAXELRXIGDXFNAYYARR

SEQ ID NO: 137 - protein, 21 aa, Artificial Sequence
  source: /note=“Description of Artificial Sequence: Synthetic peptide”
  MOD_RES (9)..(9): Any amino acid
  MOD_RES (13)..(13): Any amino acid
  MOD_RES (16)..(16): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal
IWIAQELRXIGDXFNXYYARR

SEQ ID NO: 138 - protein, 14 aa, Artificial Sequence
  source: /note=“Description of Artificial Sequence: Synthetic peptide”
  MOD_RES (4)..(4): R-octenyl alanine
  MOD_RES (5)..(5): DAB-TRIB carboxylate, DAB-Gly-TRIB carboxylate, DAB-beta-Ala-TRIB carboxylate, DAB-Linker3-TRIB carboxylate, DAB-Linker5-TRIB carboxylate, DAB-Linker7-TRIB carboxylate
  MOD_RES (11)..(11): S-pentenyl alanine
LTFXXYWAQLXSAA

SEQ ID NO: 139 - protein, 14 aa, Artificial Sequence
  source: /note=“Description of Artificial Sequence: Synthetic peptide”
  MOD_RES (4)..(4): R-octenyl alanine
  MOD_RES (11)..(11): S-pentenyl alanine
  MOD_RES (12)..(12): DAB-TRIB carboxylate, DAB-Gly-TRIB carboxylate, DAB-beta-Ala-TRIB carboxylate, DAB-Linker3-TRIB carboxylate, DAB-Linker5-TRIB carboxylate, DAB-Linker7-TRIB carboxylate
LTFXEYWAQLXXAA

SEQ ID NO: 140 - protein, 14 aa, Artificial Sequence
  source: /note=“Description of Artificial Sequence: Synthetic peptide”
  MOD_RES (1)..(1): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal
  MOD_RES (4)..(4): R-octenyl alanine
  MOD_RES (11)..(11): S-pentenyl alanine
XTFXEYWAQLXSAA

SEQ ID NO: 141 - protein, 14 aa, Artificial Sequence
  source: /note=“Description of Artificial Sequence: Synthetic peptide”
  MOD_RES (4)..(4): R-octenyl alanine
  MOD_RES (5)..(5): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal
  MOD_RES (11)..(11): S-pentenyl alanine
LTFXXYWAQLXSAA

SEQ ID NO: 142 - protein, 14 aa, Artificial Sequence
  source: /note=“Description of Artificial Sequence: Synthetic peptide”
  MOD_RES (4)..(4): R-octenyl alanine
  MOD_RES (11)..(11): S-pentenyl alanine
  MOD_RES (12)..(12): DAB-thalidomide, DAB-Gly-Thal, DAB-beta-Ala-Thal, DAB-Linker1- Thal, DAB-Linker2-Thal, DAB-Linker3-Thal, DAB-Linker4-Thal, DAB-Linker5-Thal, DAB-Linker6-Thal, DAB-Linker7-Thal, or DAB-Linker8-Thal
LTFXEYWAQLXXAA

SEQ ID NO: 143 - protein, 14 aa, Artificial Sequence
  source: /note=“Description of Artificial Sequence: Synthetic peptide”
  MOD_RES (1)..(1): DAB-VHL carboxylate, DAB-Gly-VHL carboxylate, DAB-beta-Ala-VHL carboxylate, DAB-Linker3-VHL carboxylate, DAB-Linker5-VHL carboxylate, and DAB-Linker7-VHL carboxylate
  MOD_RES (4)..(4): R-octenyl alanine
  MOD_RES (11)..(11): S-pentenyl alanine
XTFXEYWAQLXSAA

SEQ ID NO: 144 - protein, 22 aa, Artificial Sequence
  source: /note=“Description of Artificial Sequence: Synthetic peptide”
  source: /note=“N-term Acetylated”
  MOD_RES (9)..(9): (S)-2-(4-pentenyl)alanine
  MOD_RES (13)..(13): (S)-2-(4-pentenyl)alanine
  MOD_RES (22)..(22): Lys-degron
IWIAQELRXIGDXFNAYYARRX

SEQ ID NO: 145 - protein, 6 aa, Artificial Sequence
  source: /note=“Description of Artificial Sequence: Synthetic peptide”
DSGIHS